SEQUENCE LISTING (22 sequences)

SEQ ID NO: 1 - DNA, 126 bp, Artificial Sequence
  Description of Artificial Sequence Synthetic construct
gcggccgcacgcagccagagccggagcagatattaccgccagagacaaagaagtcgcagacgaaggaggcggagctgccagacacggaggagagccatgagatctcatcatcaccaccaccattaa

SEQ ID NO: 2 - DNA, 147 bp, Artificial Sequence
  Description of Artificial Sequence Synthetic construct
gcggccgcaatggccaggtacagatgctgtcgcagccagagccggagcagatattaccgccagagacaaagaagtcgcagacgaaggaggcggagctgccagacacggaggagagccatgagatctcatcatcaccaccaccattaa

SEQ ID NO: 3 - DNA, 168 bp, Artificial Sequence
  Description of Artificial Sequence Synthetic construct
gcggccgcacgcagccagagccggagcagatattaccgccagagacaaagaagtcgcagacgaaggaggcggagctgccagacacggaggagagccatgaggtgttgtcgccccaggtacagaccgagatgtagaagacacagatctcatcatcaccaccaccattaa

SEQ ID NO: 4 - DNA, 102 bp, Artificial Sequence
  Description of Artificial Sequence Synthetic construct
gcggccgcacgcagccagagccggagcagatattaccgccagagacaaagaagtcgcagacgaaggaggcggagcagatctcatcatcaccaccaccattaa

SEQ ID NO: 5 - DNA, 42 bp, Artificial Sequence
  Description of Artificial Sequence Synthetic construct
gcggccgccggcggaggaggatctcatcatcaccaccattaa

SEQ ID NO: 6 - DNA, 123 bp, Artificial Sequence
  Description of Artificial Sequence Synthetic construct
gcggccgcaatggccaggtacagatgctgtcgcagccagagccggagcagatattaccgccagagacaaagaagtcgcagacgaaggaggcggagcagatctcatcatcaccaccaccattaa

SEQ ID NO: 7 - protein, 22 aa, Artificial Sequence
  Description of Artificial Sequence Synthetic peptide
RSQSRSRYYRQRQRSRRRRRRS

SEQ ID NO: 8 - DNA, 21 bp, Artificial Sequence
  Description of Combined DNA/RNA Molecule Synthetic oligonucleotide
cguacgcggaauacuucgatt

SEQ ID NO: 9 - DNA, 21 bp, Artificial Sequence
  Description of Combined DNA/RNA Molecule Synthetic oligonucleotide
ucgaaguauuccgcguacgtt

SEQ ID NO: 10 - DNA, 21 bp, Artificial Sequence
  Description of Combined DNA/RNA Molecule Synthetic oligonucleotide
cguacgcggaauacuucgatt

SEQ ID NO: 11 - DNA, 21 bp, Artificial Sequence
  Description of Combined DNA/RNA Molecule Synthetic oligonucleotide
ucgaaguauuccgcguacgtt

SEQ ID NO: 12 - DNA, 24 bp, Artificial Sequence
  Description of Artificial Sequence Synthetic primer
tgtttgcgtctctcccaggaatca

SEQ ID NO: 13 - DNA, 24 bp, Artificial Sequence
  Description of Artificial Sequence Synthetic primer
agccctgtgcctcttcttctcatt

SEQ ID NO: 14 - DNA, 30 bp, Artificial Sequence
  Description of Artificial Sequence Synthetic primer
tgacggggtcacccacactgtgcccatcta

SEQ ID NO: 15 - DNA, 30 bp, Artificial Sequence
  Description of Artificial Sequence Synthetic primer
ctagaagcatttgcggtggacgatggaggg

SEQ ID NO: 16 - DNA, 20 bp, Artificial Sequence
  Description of Artificial Sequence Synthetic primer
cggttgaaccctacacgaag

SEQ ID NO: 17 - DNA, 19 bp, Artificial Sequence
  Description of Artificial Sequence Synthetic primer
actttccaaaggcatggtc

SEQ ID NO: 18 - DNA, 19 bp, Artificial Sequence
  Description of Artificial Sequence Synthetic primer
gcagaaagagggcgagttc

SEQ ID NO: 19 - DNA, 19 bp, Artificial Sequence
  Description of Artificial Sequence Synthetic primer
tactgaggtggcagcttcc

SEQ ID NO: 20 - DNA, 18 bp, Artificial Sequence
  Description of Artificial Sequence Synthetic primer
cctgttgtgcttctccac

SEQ ID NO: 21 - DNA, 20 bp, Artificial Sequence
  Description of Artificial Sequence Synthetic primer
atgtcaaagttcatcctgtc

SEQ ID NO: 22 - protein, 6 aa, Artificial Sequence
  Description of Artificial Sequence Synthetic 6xHis tag
HHHHHH

(19) United States
(12) Patent Application Publication
(10) Pub. No.: US 2013/0129752 A1
(43) Pub. Date: May 23, 2013

(54) TARGETED DELIVERY TO LEUKOCYTES USING PROTEIN CARRIERS

(71) Applicants: Dan Peer, Kiryat Ono (IL); Motomu Shimaoka, Brookline, MA (US); Judy Lieberman, Brookline, MA (US)

(72) Inventors: Dan Peer, Kiryat Ono (IL); Motomu Shimaoka, Brookline, MA (US); Judy Lieberman, Brookline, MA (US)

(73) Assignee: IMMUNE DISEASE INSTITUTE, INC., Boston, MA (US)

(21) Appl. No.: 12/298,361

(22) PCT Filed: Apr. 25, 2007
(86) PCT No.: PCT/US07/09975

§ 371 Date: Oct. 24, 2008

Related U.S. Application Data
(60) Provisional application No. 60/794,817, filed on Apr. 25, 2006.

(51) Int. Cl. A61K 39/395; C12N 5/06; A61K 35/12; C07K 16/18

(52) U.S. Cl. 424/178.1; 530/391.7; 435/375; 424/93.71

(57) ABSTRACT

Disclosed herein are is a leukocyte-selective delivery agent comprising, a targeting moiety that selectively binds LFA-I, a protein carrier moiety covalently linked to the targeting moiety, and a therapeutic agent associated with the carrier moiety. The delivery agent may be further selective for activated leukocytes, wherein the targeting moiety selectively binds LFA-I in its activated conformation. The targeting moiety comprises an antibody or functional fragment thereof, such as an scFV. Examples of antibodies or fragments thereof which selectively bind LFA-I activated conformation bind to the locked open I domain of LFA-I, or binds to the leg domain of the β2 subunit of LFA-I ((ILP2)—The antibody or functional fragment thereof may alternatively bind non-selectively to both low affinity and high affinity LFA-I. Examples of a non-protein carrier are a basic polypeptide such as protamine or a functional fragment thereof. One such fragment is RSQSRSRYYRQRQRSRRRRRRS. The therapeutic agent may comprise one or more of a nucleic acid, a small molecule, a polypeptide, and an antibody or functional fragment thereof. An example of a nucleic acid delivery agent comprises an RNA interference molecule. Examples of RNA interference molecules are siRNA, dsRNA, StRNA, shRNA, miRNA, and combinations thereof. Specific siRNAs are provided. Other examples of a nucleic acid delivery agent are a small RNA, an antagomir, an LNA, and an antisense oligonucleotide. Methods for leukocyte-selective delivery, or activated leukocyte-selective delivery in vivo, in vitro and ex vivo are also provided.

### RELATED APPLICATIONS

This application is an International application, which claims the benefit of priority under 35 U.S.C. §119(e) of U.S. Provisional Application Ser. No. 60/794,817 filed on Apr. 25, 2006, the contents of which are incorporated herein by reference in its entirety.

### GOVERNMENT SUPPORT

This invention was supported, in part, by National Institutes of Health (NIH) Grant No. A1063421. The government of the United States has certain rights to the invention.

### BACKGROUND OF THE INVENTION

The migration of leukocytes through the body and the various lymphoid organs is an essential element of the immune system. While circulating in blood or lymphatic vessels, leukocytes are in a resting and low adhesive state. However, when leukocytes are stimulated by signals from the immune system such as exposure to an immune complex or a chemokine gradient, their integrin adhesion receptors become activated. The activation of the integrins is essential for the many functions of leukocytes.

Such functions are, for example, binding to antigen-presenting cells, recirculation through lymph nodes and migration out of the vasculature and through the extracellular matrix to sites of inflammation. The integrin activation needs to be tightly regulated as inappropriate leukocyte adhesion leads to significant injury of normal tissues.

Leukocytes express a specific subset of the integrin family, the β₂ integrins, of which four members are currently known. They have a common β₂ chain (CD 18) but different α subunits (α_{L}/CD11a, α_{M}/CD11b, αₓ/CD11c, and αD/CD11d) (Gahmberg et al., 1997, Eur. J. Biochem 245:215-232). The α subunits contain a conserved 200-residue A or I domain, which is essential for binding of most ligands. The crystal structures of I domains from the α_{L} and α_{M} subunits indicate the presence of a cation binding site called the metal ion-dependent adhesion site (MIDAS). Amino acid substitutions in this site abrogate ligand binding (Huang and Springer, 1995, J. Biol. Chem. 270:19008-19016; Kamata et al., 1995, J. Biol. Chem. 270, 12531-12535).

The major ligands of these integrins, the ICAMs, belong to the immunoglobulin superfamily, and five ICAMs with slightly different binding specificities have been described. The expression of ICAM-1 on endothelial cells is subject to stimulation by inflammatory cytokines, which enhances the β₂ integrin-mediated adhesion of leukocytes on endothelial cells. LFA-1 (α_{L}β₂) dependent ICAM-1 stimulation has been implicated in leukocyte adhesion, aggregation and transendothelial migration.

Inhibition of LFA-1/ICAM-1 binding has potential therapeutic benefits relating to blocking allograft rejection, including cardiac, renal and thyroid allografts (Isobe et al., Science, 255:1125, 1992; Stepkowski et al., 1994, J Immunol., 144:4604; Cosimi et al., 1990, J. Immunol, 144:4604; Nakakura et al., 1993, Transplantation, 55:412; Talento et al., Transplantation, 55:418, 1993), bone marrow transplants (Tibbetts et al., Transplantation, 68:685, 1999; Cavazzana-Calvo et al., Transplantation, 59:1576, 1995) T-cell mediated sensitization reactions (Ma et al., Cell Immunol., 15:389, 1994; Cumberbatch et al., Arch. Dermatol. Res., 288:739, 1996), diabetes (Hasegawa et al., Int. Immunol., 6:831, 1994), rheumatoid arthritis (Davis et al., J. Immunol., 154:3525, 1995; Kavanaugh et al., Arthritis Rheum., 37:992, 1994), and artherosclerosis (Kawamura et al. Circ J 68:6-10, 2004). Expression of ICAM-1 by keratinocytes is also implicated in the etiology of psoriasis, and inhibition of LFA-1/ICAM-1 binding presents a possible point of therapeutic intervention (Servitje et al., J Cutan. Pathol., 23:431, 1996). Thus the peptide compositions of the present invention may be used in treatment of the above conditions and more generally in any condition T-cell mediated condition wherein T-cells are activated via interaction of LFA-1 and ICAM-1.

Anti-integrin therapy using blocking antibodies is a promising anti-inflammatory remedy [61-64]. As integrins require activation by intracellular signaling cascades for binding to ligands, the signaling molecules that induce integrin activation are novel therapeutic targets for the treatment of autoimmune and inflammatory diseases. Talin and Rap-1 have emerged as important signaling molecules for integrin activation. Talin is a major cytoskeletal protein that co-localizes with activated integrins and binds to integrin β cytoplasmic domains [65]. Talin is a component of focal adhesions and provides a link between integrins and the cytoskeleton. Talin directly interacts with the cytoplasmic tails of and consequently activates the β1, β2, and β3 integrins [66-68]. siRNA silencing of talin inhibits LFA-1-mediated lymphocyte adhesion in vitro [67]. The small GTPase, Rap1, is a potent activator of leukocyte integrins and enhances the adhesive activity of LFA-1 when stimulated by the T cell receptor (TCR) or chemokines [60]. Defective Rap-1 activation has been found in leukocyte adhesion deficiency type-III, where cell adhesion by LFA-1 and VLA4 are impaired [69]. Involvement of talin and Rap-1 in the activation of multiple integrins will enhance the inhibition of leukocyte accumulation at site of inflammation, as leukocyte migration to inflammatory tissues involves multiple integrins [70]. In addition to molecules involved in integrin signaling, proinflammatory cytokines [71] and transcription factors that activate inflammatory mediators such as NF-κB [72,73] will be potential targets for AL-57-directed siRNA delivery and silencing.

Therefore, it will be useful to develop activated leukocyte-selective anti-inflammatory therapeutics via interfering with integrin signaling and inflammatory cascades with siRNAs or other therapeutic agents. Potential targets of this novel activated leukocyte selective therapeutic approach include autoimmune diseases, graft-versus-host disease, and septic shock.

### SUMMARY OF THE INVENTION

Aspects of the present invention relate to a leukocyte-selective delivery agent comprising, a targeting moiety that selectively binds LFA-1, a protein carrier moiety covalently linked to the targeting moiety, and a therapeutic agent associated with the carrier moiety. The delivery agent may be further selective for activated leukocytes, wherein the targeting moiety selectively binds LFA-1 in its activated conformation.

In one embodiment, the targeting moiety comprises an antibody or functional fragment thereof, such as a scFV. The antibody or functional fragment thereof may bind to the locked open I domain of LFA-1, or binds to the leg domain of the β₂ subunit of LFA-1 (α_{L}β₂). In another embodiment, the antibody or functional fragment thereof binds non-selectively to both low affinity and high affinity LFA-1. In one embodiment the protein carrier moiety comprises a basic polypeptide. The protein may comprise protamine or a functional fragment thereof. One such fragment is RSQSRSRYYRQRQRSRRRRRRS (SEQ ID NO: 7). The therapeutic agent may comprise one or more of a nucleic acid, a small molecule, a polypeptide, and/or an antibody or functional fragment thereof. An example of a nucleic acid delivery agent comprises an RNA interference molecule. Examples of RNA interference molecules are siRNA, dsRNA, stRNA, shRNA, miRNA, and combinations thereof. In one embodiment, the therapeutic agent comprises CCR5-siRNA, ku70-siRNA, CD4-siRNA or cyclin-D1-siRNA. Other examples of a nucleic acid delivery agent are a small RNA, an antagomir, an LNA, and an antisense oligonucleotide.

Aspects of the present invention also relate to a method for leukocyte-selective delivery, or activated leukocyte-selective delivery, comprising, administering to a subject an activated leukocyte-selective delivery agent described herein. Administration is to contact the delivery agent with activated leukocytes of the subject, to thereby selectively deliver the therapeutic agent to activated leukocytes of the subject. In one embodiment, the subject has inappropriate leukocyte activation prior to administration of the delivery agent.

Aspects of the present invention also relate to a method for leukocyte-selective delivery comprising providing a leukocyte-selective delivery agent described herein, and contacting the delivery agent to a population of cells comprising leukocytes, to thereby selectively deliver the therapeutic agent to leukocytes in the population of cells. In one embodiment, the population of cells is obtained from a subject, and contacting is performed in vitro. The method may further comprise administering the population of cells which contacted with the delivery agent, to the subject.

### BRIEF DESCRIPTION OF THE DRAWINGS

FIGS. 1A-1D are graphical representations of data which indicate binding of AL-57 to LFA-1 on the cell surface in K562 transfectants expressing LFA-1 (FIG. 1A) and T-lymphocytes (FIGS. 1B & 1C). FIG. 1A is a histogram of data indicating LFA-1 in K562 cells either in the inactive (Mg2+/Ca2+) or the active (Mg2+/EGTA/CBRLFA-1/2) states were stained by AL-57 (closed histograms) or isotype IgG (open histograms). Mean fluorescent intensity (MFI) values are shown. FIGS. 1B & 1C shows that T-lymphocytes were activated either by PMA (100 nM) or CXCL-12 (SDF-1, 100 ng/ml), and stained with AL-57, another activation-dependent mAb KIM127, or a control activation-insensitive TS2/4. FIG. 1B is a collection of nine representative FACS histograms. Background binding by isotype control IgG is shown by open histograms. MFI values are shown. FIG. 1C is a bar graph that shows the number of epitopes expressed on cells that was determined by IFC using Quantum Simply Cellular beads (Bangs Lab). T-cells were incubated with PMA and SDF-1α for 20 min at 37° C. Fab AL-57, KIM127, and TS2/4 were added 2 min prior to immediate fixation by adding formaldehyde. Cells and Quantum Simply Cellular beads were then incubated with FITC conjugated secondary mAb. FIG. 1D is a line graph of data that indicates inhibition of LFA-1-ICAM-1 interaction by AL-57. K562 expressing high-affinity LFA-1 was incubated with ICAM-1-Fcα/IgA-FITC in the presence of AL-57 or activation-independent LFA-1 mAb MHM24. Bound ICAM-1 was measured by IFC and expressed as MFI.

FIG. 2 is a bar graph of data indicative of silencing CD4 in PBMC. CD4-siRNA condensed by protamine was entrapped in AL-57-, TS1/22, or IgG-NPs. The efficiency of entrapment was measured as described. Cells (2×10⁵ cells in 0.5 mL) were given 1000 pmol CD4-siRNA with or without carriers, and cultured for 60 hrs either in resting (Mg/Ca) or activating (Mg/EGTA/CBRLFA-1/2) conditions. A faction of CD4+ cells was determined by IFC. Data are expressed as percentage of CD4+ population relative to MOCK-treated sample.

FIG. 3 is a set of twelve histograms of data indicative of silencing of CD4 by siRNA in CD4+ T-cells, in which LFA-1 is activated by physiologic inside-out signaling. Cells were activated with immobilized agonists shown in the figure. Cells were treated with 1000 pmol CD4-siRNA incorporated in AL-57- or IgG-NP. Expression of CD4 was determined by IFC. MFI values are shown.

FIG. 4 is a set of twelve skatchard plots of data indicative of CD4 silencing by AL-57-PF in PBMC. Cells (2×10⁵ cells in 0.5 mL) were given CD4-siRNA complexed with AL-57-PF or PEI. Cells were cultured for 60 hrs either in resting (Mg/Ca) or activating (Mg/EGTA/CBRLFA-1/2) conditions, and subjected to IFC analyses for determining the faction of CD4+ cells. Data are expressed as percentage of CD4+ population relative to MOCK-treated sample. The amount of CD4-siRNA used was shown in parentheses.

FIGS. 5A-5B are graphical representations of data which indicate silencing of Ku70 by AL-57-PE in T-cells. FIG. 5A is a collection of eleven histograms. FIG. 5 B is a line graph. T-cells were treated with 1000 pmol Ku70-siRNA complexed with AL-57- or non-binding ML39-PF in the presence or absence of activation (Mg/EGTA/CBRLFA-1/2). Expression of intracellular Ku70 was determined by IFC after permealization. MFI values are shown. FIG. 5B indicates dose dependent Ku70 silencing by AL-57-PF in activated T-cells. NT, not tested.

FIG. 6 is a bar graph of data which indicates silencing by AL-57-PF in CD4 T-cells activated by physiologic outside-in signaling to LFA-1. IL-2-treated T-cells were treated with 1000 pmol CD4-siRNA complexed with AL-57-PFor ML39-PF in the presence of immobilized agonists shown. Expression of CD4 was determined by IFC. MFI values are shown.

FIGS. 7A-7C are graphical representations of data which indicate successful reconstitution of hu PBL in immunodeficient mice. FIG. 7A is a bar graph of levels of engraftment monitored by the presence of CD45+ human lymphocytes in peripheral blood. FIG. 7B is a set of four representative FACS plots at day 14th, indicating both CD4+ and CD8+ human T-cell populations in peripheral blood. FIG. 7C is a bar graph of levels of engraftment of hu CD45+ cells in tissues. *Rag-PBL, Rag^{-/-}IL2rγ^{-/-}-hu-PBL; ^{‡}SCID-PBL, NOD/Lt-scid IL2rγ^{null}-hu-PBL.

FIG. 8 is a photo of a SDS-PAGE gel. AL-57-PF, TS1/22-PF, and the respective targeting moiety without protamine were fractioned by SDA-PAGE. Each protein product migrated at the expected positions.

FIGS. 9A-9B are representative histograms of immunofluorescence flow cytometry. The data show binding of AL57-PF and TS1/22-PF to fresh PBMC. Human primary PBMC were stained with Alexa 488-conjugated-AL-57-PF, TS1/22-PF and ML39-PF (isotype control). Staining was done at 20 μg/mL, for 30 min, at 37° C. in active and näive conditions. FIG. 9A näive PBMC were supplemented with 1 mM CaCl₂ and MgCl₂ in their media. FIG. 9B PBMC were activated using 5 mM MgCl₂, 1 mM EGTA and 10 μg/mL of CBRLFA1/2 (activating antibody). Solid black curve—ML39-PF (isotype control) overlaid by dash curve—AL-57-PF (conformational sensitive) and dot curve—TS1/22-PF (conformational insensitive).

FIG. 10 is a line graph of data indicative of dose dependent binding to activated PBMC. Activated PBMC (5 mM MgCl₂, 1 mM EGTA and 10 μg/mL of CBRLFA1/2 (activating antibody)) were stained with increasing doses of ML39-PF (isotype control), AL-57-PF, and TS1/22-PF. All fusion proteins were labeled with Alexa 488 dye (Molecular probes) as detailed in the experimental section. The figure represents an average of 4 independent experiments. Error bars represent the standard deviation.

FIG. 11 is a bar graph of data indicative of sustained activation of IL-15 cultured lymphocytes using immobilized agonists and activation by CBRLFA-1/2 (10 μg/mL), 5 mM MgCl₂ and 1 nM EGTA. Binding of AL-57-PF, TS1/22-PF and ML39-PF (isotype control) to IL-15 cultured lymphocytes was monitored. All fusion proteins were labeled with Alexa 488 dye (Molecular probes) as detailed in the experimental section. Staining was done at 20 μg/mL, for 15 min, at 37° C. Activation by immobilized agonists at different time points is presented. Immobilized α-CD3 (10 μg/mL); Immobilized α-CD3/CD28 (10 μg/mL each); and Immobilized CXCL-12 (5 μg/mL). The results are presented as AL-57⁺ active conformation (% of TS1/22) in different time points using the formula:

AL-57⁺ active conformation (% of TS1/22)=[AL-57-PF mean fluorescence intensity (MFI)−ML39-PF MFI]/[TS1/22-PF MFI-ML39-PF MFI]×100

The results are an average of 3 independent experiments. The error bars represent the standard deviation between the experiments.

FIG. 12 is a line graph of data that indicates AL-57-PF and TS1/22-PF can bind approximately 5 molecules of cy3 labeled-siRNA. A fixed amount of Cy3-siRNA was incubated with varying amounts of fusion proteins (either AL-57-PF or TS1/22-PF) bound to anti-protamine coupled beads and binding of bead bound cy3-siRNA was measured by fluorescence intensity compared to a standard curve.

FIGS. 13A-13B are a bar graph and a line graph, respectively, of data indicative of silencing of CD4 in Fresh PBMC. FIG. 13A näive (resting) PBMC or Active PBMC (activated by CBRLFA-1/2/Mg/EGTA) were used immediately after PBMC isolation (as detailed in the Examples section below). CD4-siRNA (1000 pmol) was complexed with various delivery systems (ML39-PF, AL-57-PF, or TS1/22-PF) at a 1:5 ratio (as presented in FIG. 5) for 30 min at room temperature before transfecting the näive or activated PBMC. PEI (Gen500) or Oligofectamine™ (Invitrogen) were used according to manufacture's guidelines. Silencing was observed after 60 hours using FITC-labeled anti-CD4. The results are presented as % of CD4 expression. Average±SD are presented from 3 independent experiments. ** represents p<0.01; *** represents p<0.001 by two tailed student's t test. FIG. 13B active PBMC (activated by CBRLFA-1/2/Mg/EGTA) were transfected with various amounts of CD4-siRNA as detailed in the experimental section. ML39-PF served as isotype control and showed no reduction in silencing. Expression of LFA-1 in its active conformation requires for targeting to conformational sensitive delivery system (AL-57-PF) in order to silence CD4. TS1/22-PF, the conformational insensitive delivery system, was more effective than AL-57-PF, and both carriers reached plateau at 1000 pmol of siRNA. The results are presented as average of 3 independent experiments and the error bars are the standard deviation between these experiments.

FIG. 14A-14B are line graphs of data indicative of silencing Ku70 in PBMC. The delivery systems were complexed with Ku70-siRNA and transfected PBMC as described in the experimental section. FIG. 14A was done with näive PBMC. FIG. 14B was done with activated PBMC (activated by CBRLFA-1/2/Mg/EGTA). Data is presented as average of 4 independent experiments and the error bars are the standard deviation between these experiments. siRNA delivered by TS1/22-P in both the active and näive cells is plateau at 2000 pmol. SiRNA delivered by AL-57-PF is plateau at approximately 1000 pmol in the activated cells.

FIG. 15 is a set of 18 histograms of data indicative of silencing of Ku70 in IL-15 cultured lymphocytes. Immobilized agonists were used to activate the lymphocytes as detailed in the experimental section. Ku70-siRNA was complexed with the delivery systems at an amount of 1000 pmole as detailed in the experimental section. Representative histograms are presented. Mean fluorescence Intensity (MFI) is listed in each histogram.

FIG. 16 is a bar graph of data indicative of inhibited Proliferation of IL-15 cultured lymphocytes on immobilized agonists by cyclin-D1-siRNA delivered by AL-57-PF and separately by TS1/22-PF. 11-15 cultured lymphocytes were grown on plastic dishes immobilized with agonists as detailed in the experimental section. 1000 pmole of Cyclin-D1-siRNA was complexed to the delivery systems and transfected the IL-15 cultured lymphocytes as described in the experimental section. MTT assay was preformed after 72 hours post transfection. The results are presented as the mean O.D. 570 nm±standard deviation from 3 independent experiments. * represent p<0.05; ** represent p <0.01 by two tailed student's t test.

FIG. 17A-D are a collection of graphical representations of data which indicate selective targeting of siRNAs to PBMC expressing HA LFA-1 by AL-57-PF.

FIG. 18A is a set of two side by side line graphs which indicates activation-independent binding of TS1/22-PF and activation dependent binding of AL-57-PF. PBMC were either unstimulated (1 mM MgCl₂, 1 mM CaCl₂) or stimulated with 5 mM MgCl₂, 1 mM EGTA, and 10 ug/ml CBRLFA-1/2 to activate LFA-1. FIG. 17B is a set of two side by side bar graphs that indicate selective delivery of Cy2-siRNA (1 mmol) to stimulated or unstimulated PBMC, measured 6 hr after treatment. The LFA-1 antibody fusion proteins selectively delivered siRNAs to T lymphocytes (stained with CD3), B lymphocytes (CD19), monocytes (CD14), and dendritic cells (CD11c). FIG. 17C is a set of two side by side line graphs of data which indicates silencing of Ku70 in pbmc. Ku70 expression was measured 3 d after treatment with Ku0-siRNA, delivered as indicated in the Examples section below. FIG. 17D is a bar graph of data which indicates silencing of CCR5 in T lymphocytes. Memory T lymphocytes were treated for 3 d in the presence or absence of LFA-1 acivating antibody with 1 nmol of CCR5-siRNA, delivered as indicated. Expression of CCR5 mRNA relative to B-actin mRNA was measured by uantitative PCR.

FIGS. 18A and B are graphical representations of data indicative of siRNA delivery to PBMC by LFA-1 antibody-fusion proteins. Cells were unstimulated or stimulated with Mg/EGTA plus an activating mAb CBRLFA-1/2. FIG. 18A is a bar graph. As seen in FIG. 18A, stimulation with CBRLFA-1/2 did not affect LFA-1 expression on any subset of cells. FIG. 18B is a set of two representative flow cytometry histograms indicative of binding of Alexa 488-conjugated scFv-PF (20 μg/ml). Conformation-dependent AL-57-PF (solid lines) binds only to stimulated cells, while conformation-insensitive TS1/22-PF (dashed lines) binds to either unstimulated or stimulated cells and the control ErbB2 fusion protein ML39-PF (dotted lines) binds to neither.

FIGS. 19A and B is a set of two bar graphs indicative of siRNA-mediated silencing of Ku70 in PBMC (FIG. 19A) or CD4 in CD4⁺ lymphocytes (FIG. 19B). Cells that were either unstimulated or stimulated with Mg/EGTA plus CBRLFA-1/2, were treated for 3 d with 1 nmol Ku70-siRNA or CD4-siRNA complexed with the indicated delivery reagents, and expression of Ku70 or CD4 was measured by flow cytometry. Data are mean SD of three independent experiments, normalized to expression of mock-treated cells.

FIGS. 20A, B, C and D are each skatter plots. The data collectively indicate selective silencing of Ku70 in mixed populations of K562 cells transfected to express LFA-1. CMTMR-labeled CBRLFA-1/2-activated cells, expressing HA LFA-1. were cocultured ith the unlabeled cells treted with an LFA-1 nonactivating antibody that express low-affinity LFA-1. Three days after treatment with 1 mmol of Ku70-siRNA delivered as indicated, the cocultures were analyzed for Ku70 silencing. AL57-PF-delivered siRNAs silence only the labeled activated cells (FIG. 20D), whereas TS-1/22-PF-delivered siRNAs silenct Ku70 in both poulations (FIG. 20C).

FIG. 21 is a set of three skatter plots. These dot plots serve as additional controls to the experiments which generated FIG. 21 confirm the specificity of Ku70-siRNA delivered with LFA-1 antibody fusion proteins in heterogeneous populations. CMTMR-labeled, CBRLFA-1/2-activated cells that express high affinity LFA-1 were cocultured with unlabeled, TS2/4-unactivated cells that express low-affinity LFA-1. Three days after treatment with 1 nmol Ku70-siRNA (FIGS. 21A and B) or luciferase-siRNA (FIG. 21C) delivered as indicated, the cocultures were analyzed for Ku70 expression.

FIGS. 22A, B, C, and D are graphical representations of data. Collectively the data indicate persistent physiological stimulation of memory T cells activates sustained AL-57-PF binding and siRNA delivery. FIG. 22A-C are line graphs of data which indicate the kinetics of affinity up-regulation of LFA-1 after activation of T cells. Cells stimulated for the indicated times with immobilized CXCL12 or anti-CD3 were analyzed for binding of Alexa488-labeled fusion proteins. FIG. 22D is a collection of 12 histograms of data indicative of activation-dependent silencing of Ku70 in T cells measured 3 d after treatment with 1 nmol of Ku70-siRNA delivered by scFv-PF. Mean fluorescence intensities (MFI) of representative histograms are shown.

FIG. 23 is a bar graph of data indicative of selective inhibition of proliferation by AL-57-PF-delivered cyclin D1-siRNA to activated T cells. Prolifertion was assayed by 3-(4,5-dimethylthiazol-2-yl)-2,5-diphenyl tetrazolium bromide (MTT) incorporation 3 d after treatment with or without immobilized activating antibodies, combined with cyclin D1 or control siRNA complexed with scFv-PF fusion proteins, TS1/22 scFv, protamine, or medium. Silencing cyclin D1 using TS1/22-PF stopped proliferation of all T cells, whereas inhibition of proliferation using AL-57-PF required cell activation. *, P<0.03; **, P<0.01.

FIG. 24 is a bar graph of data indicative of knockdown of cyclin D1 in T cells. IL-15-cultured T cells were treated for 60 h with 1 nmol siRNA mixed with protamine, TS1/22 scFv (TS1/22), or antibody-protamine fusion proteins (ML39-PF, TS1/22-PF, or AL-57-PF) in the presence of immobilized antibodies: 5 μg/ml anti-CD3 (CD3); a combination of 5 μg/ml anti-CD3 and 5 μg/ml anti-CD28 (CD3/CD28); or 5 μg/ml isotype control IgG (MOCK). Cells were fixed and permeabilized, and stained with Cyclin-D1-FITC mAb (clone DCS-6, Santa Cruz Biotechnology). Data are mean SD of three independent experiments and shown as a percentage of the mean fluorescent intensity of cyclin D1 in untreated cells. *P<0.05, **P<0.01. Silencing correlated with the specificity of the antibody and correlated with suppression of cellular proliferation shown in FIG. 23

FIGS. 25A, B, and C are graphical representations of data which indicate anti-LFA-1 scFv fusion protein-siRNA complexes do not activate lymphocytes or induce IFN responses in PBMC. FIGS. 25A and B are sets of two histograms. Cell surface expression of the activation markers CD69 and CD25 was measured by flow cytometry 2 d following treatment of PBMC with 1 nmol luciferase-siRNA complexed with indicated scFv-PF (dashed lines), siRNA alone (thick lines), or PHA (thin lines). FIG. 25C is a bar graph which shows expression of IFN responsive genes relative to β-actin analyzed by quantitative RT-PCR in CBRLFA-1/2-activated PBMC treated with luciferase-siRNA delivered as indicated. Poly (I:C) and LPS were used as positive controls to induce IFN responses. The siRNA complexes did not induce either cellular activation or an IFN response.

### DETAILED DESCRIPTION OF THE INVENTION

Aspects of the present invention relate to cell type specific delivery of an agent to a cell via binding of an integrin exclusively or primarily expressed on that cell type (e.g. within a mixed population of cells that contains non-target cells). Embodiments of the present invention are directed to a leukocyte-selective delivery agent that selectively targets leukocytes by way of selective binding to an integrin which is exclusively or primarily expressed on leukocytes (herein referred to as a leukocyte integrin). Other embodiments of the present invention are directed to activated cell-selective delivery agents that selectively target activated cells by way of selective binding to an integrin in its activated conformation that is exclusively or primarily expressed on the activated target cell. One example of this is an activated leukocyte selective delivery agent that selectively targets activated leukocytes by way of selective binding to a leukocyte integrin in its active conformation (e.g. high-affinity conformation). Other embodiments of the present invention are directed to inactive cell-selective delivery agents that selectively target inactive cells by way of selective binding to an integrin in its inactive conformation that is exclusively or primarily expressed on the inactive target cell. One example of this is an inactive leukocyte selective delivery agent that selectively targets inactive leukocytes by way of selective binding to a leukocyte integrin in its inactive conformation (e.g. low-affinity conformation).

The delivery agent of the present invention comprises three components: a targeting agent or targeting moiety that selectively binds to a target cell type, e.g., leukocytes; a carrier moiety that is associated (e.g. covalently) with the targeting moiety; and a therapeutic agent that is associated with the carrier moiety. The targeting moiety serves to effect selective transport of the carrier moiety to the target cell type, wherein the carrier moiety delivers the therapeutic agent to the target cell type. The targeting moiety is attached to the carrier moiety (e.g. via chemical conjugation, cross-linking or fusion protein. Therapeutic agents, e.g., siRNAs, are associated with the carrier particles. In one embodiment, the targeting moiety is an antibody selective for the active conformation of the integrin LFA-1. In one embodiment, the carrier particle is a protamine. In one embodiment, the carrier moiety is an immunoliposome.

The selective delivery agent is used in methods described herein to selectively delivery an agent (e.g. a therapeutic agent) to the targeted cell type (e.g. in a mixed population of cells). As such, aspects of the present invention relate to methods for selective delivery comprising, contacting target cells (e.g. in vivo, in vitro, ex vivo) with a delivery agent described herein.

The leukocyte delivery agent is used in methods described herein to selectively deliver an agent to leukocytes as the target cells. As such, aspects of the present invention relate to methods for leukocyte-selective deliver of a therapeutic agent, comprising contacting the delivery agent to the target cells (e.g. in a mixed population of cells).

The activated (or inactive) leukocyte selective delivery agent is used in methods described herein to selectively delivery an agent to the activated (or inactive) leukocytes as the target cells. As such, aspects of the present invention relate to methods for activated (or inactive) leukocyte-selective delivery of a therapeutic agent comprising, contacting the deliver agent to the activated (or inactive) leukocyte target cells (e.g. in a mixed population of cells).

In one embodiment, contacting is done in vivo, and comprises administering the delivery agent to a subject by a method suitable to promote contact with the delivery agent to the target cells within the subject. For example, if the cells are located in the circulatory system, suitable contacting would be intra venous administration, although other forms of administration would also promote contact of circulatory system cells. Additional suitable forms of contacting are discussed in more detail below.

Use of an activated leukocyte selective delivery agent will target a therapeutic to activated leukocytes. Targeted delivery to activated leukocytes serves as means for therapy of a variety of disease conditions which involve inappropriately activated leukocytes (e.g. anti-inflammatory therapy). Because this embodiment of the present invention selectively targets only activated leukocytes, therapeutic intervention can be designed to affect only aberrantly activated cells without perturbing normal immune homeostasis.

The present invention relates to a method to deliver therapeutics, such as small molecule drugs, nucleic acid-based therapeutics, and peptide-based therapeutics, by contacting leukocytes with a delivery agent. The delivery agent comprises a carrier moiety which is linked or associated with one or more of these therapeutics (therapeutic agents) and is also linked or associated with one or more targeting moieties. The targeting moieties specifically target leukocytes by way of interaction with an integrin. The targeting moieties can specifically target activated leukocytes by way of selective recognition of the active conformation of the integrin (e.g. the α subunit of the leukocyte integrins, such as LFA-1 and MAC-1).

### Targeting Agent

The term “targeting agent” or “targeting moiety,” used interchangeably herein, refer to an agent that homes in on or preferentially associates or binds to a particular tissue, cell type, receptor, infecting agent or other area (or target) of interest. A targeting agent suitable for use in the present invention must have sufficient binding affinity for the target under physiological conditions to selectively deliver the delivery agent to the appropriate cell type by the desired delivery method (e.g. in vivo, in vitro, ex vivo). Examples of a targeting agent include, but are not limited to, an oligonucleotide, an antigen, an antibody or functional fragment thereof, a ligand, a receptor, one member of a specific binding pair, a polyamide including a peptide having affinity for a biological receptor, an oligosaccharide, a polysaccharide, a steroid or steroid derivative, a hormone, e.g., estradiol or histamine, a hormone-mimic, e.g., morphine, or other compound having binding specificity for a target. In the methods of the present invention, the targeting agent promotes transport or preferential localization of the delivery vehicle of the present invention to the target of interest, i.e., activated leukocytes.

A delivery agent of the present invention may utilize one or more different targeting agents. A plurality of targeting agents, each with their own binding target, on a particular deliver agent can be used to facilitate delivery to a broader spectrum of cell types (more than one cell type), or alternatively, to narrow the target cell type.

Antibodies and functional fragments or derivatives thereof which exhibit the desired binding activity (specifically bind the desired cell surface antigen) are useful targeting agents, or components thereof. As used herein, an “antibody” or “functional fragment” of an antibody encompasses antibodies and derivatives thereof which exhibit the desired specific binding activity. This includes, without limitation, polyclonal and monoclonal antibody preparations, as well as preparations including hybrid or chimeric antibodies, such as humanized antibodies, altered antibodies, antibody fragments such as F(ab′)₂ fragments, F(ab) fragments, Fv fragments, single domain antibodies, dimeric and trimeric antibody fragment constructs, minibodies, and functional fragments thereof which exhibit immunological binding properties of the parent antibody molecule and/or which bind a cell surface antigen.

One aspect of the present invention relates to compositions and methods where the target cell population is leukocytes. In one embodiment, the target is all leukocytes regardless of their activation state. This is accomplished by targeting a cell surface molecule, e.g. an integrin molecule, which is specifically/exclusively expressed on leukocytes. Examples of such integrin molecules are LFA-1 and Mac-1. A targeting moiety which preferentially associates or binds to the integrin as it is expressed on all leukocytes will selectively bind to all contacted leukocytes. Such a targeting moiety will associate with the integrin molecule in a way that will not be affected by conformational changes the integrin molecule exhibits as a function of its activation state. For instance, an antibody which recognizes the integrin molecule in both the active and inactive conformation and binds them equally well would serve as an acceptable leukocyte activation insensitive targeting moiety.

One example of an activation-insensitive antibody is TS1/18. This is a mouse anti-human monoclonal antibody to the beta subunit of human LFA-1 (aLb2). TS1/18 binds both the inactive and active LFA-1 equally. It was generated in mice through convention hybridoma methods (Tonneson et al., (1989) J. Clin. Invest. 83(2): 637-46).

The conformation adopted by intergrins on the cell surface is reflective of the activation state of the cell in many cell types. In these cell types, inactive cells have integrins in an inactive conformation (that does not bind ligand), whereas active cells have integrins which have changed shape (conformation) to allow ligand binding. This difference in conformation can be exploited to selectively deliver the delivery agent of the present invention to a cell in a desired activation state. More specifically, a targeting moiety which selectively binds a specific conformation (active or inactive) will selectively target the delivery agent to cells of the corresponding activation state. This concept can be exploited to not only target specific activation states of leukocytes, but other cell types as well which exhibit different activation states (by identifying appropriate targets on the target cells). One can selectively target inactive cells or active cells by generating and/or using targeting moieties (e.g. antibodies or functional fragments thereof) which specifically recognize the desired integrin conformation.

Integrins exist on cell surfaces in an inactive conformation that does not bind ligand. Upon cell activation, integrins change shape (conformation) and can bind ligand. It has been proposed that the intramolecular conformational changes accompanying integrin activation increase integrin affinity for ligand. After activation, integrins bind in a specific manner to protein ligands on the surface of other cells, in the extracellular matrix, or that are assembled in the clotting or complement cascades. Integrins on leukocytes are of central importance in leukocyte emigration and in inflammatory and immune responses. Over 20 different integrin heterodimers (different α and β subunit combinations) exist that are expressed in a selective fashion on all cells in the body. Ligands for the leukocyte integrin Mac-1 (αMβ2) include the inflammation-associated cell surface molecule ICAM-1, the complement component iC3b, and the clotting component fibrinogen. Ligands for the leukocyte integrin LFA-1 (αLβ2) include ICAM-1, ICAM-2, and ICAM-3. Antibodies to leukocyte integrins can block many types of inflammatory and auto-immune diseases, by, e.g., modulating, e.g., inhibiting, for example, cell to cell interactions or cell to extracellular matrix interactions.

The active conformation of the integrin, e.g., LFA-1, is associated with a conformational change in the I-domain. The N-terminal region of the integrin α subunits contains seven repeats of about 60 amino acids each, and has been predicted to fold into a 7-bladed β-propeller domain (Springer, T A (1997) Proc Natl Acad Sci USA 94:65-72). The leukocyte integrin α subunits, the α1, α2, α10, α11, and αE subunits contain an inserted domain or I-domain of about 200 amino acids (Larson, R S et al. (1989) J Cell Biol 108:703-712; Takada, Y et al. (1989) EMBO J 8:1361-1368; Briesewitz, R et al. (1993) J Biol Chem 268:2989-2996; Shaw, S K et al. (1994) J Biol Chem 269:6016-6025; Camper, L et al. (1998) J Biol Chem 273:20383-20389). The inserted or I-domain is predicted to be inserted between β-sheets 2 and 3 of the β-propeller domain. The I domain of the α subunit is an allosteric mediator of ligand binding. The three dimensional structure of the αM, αL, α1 and α2 I-domains has been solved and shows that it adopts the dinucleotide-binding fold with a unique divalent cation coordination site designated the metal ion-dependent adhesion site (MIDAS) (Lee, J-O, et al. (1995) Structure 3:1333-1340; Lee, J-O, et al. (199S) Cell 80:631-638; Qu, A and Leahy, D J (1995) Proc Natl Acad Sci USA 92:10277-1028 1; Qu, A and Leahy, D J (1996) Structure 4:931-942; Emsley, J et al. (1997) J Biol Chem 272:28512-28517; Baldwin, E T et al. (1998) Structure 6:923-935; Kallen, J et al. (1999) J Mol Biol 292:1-9). The C-terminal region of the αM subunit has been predicted to fold into a β-sandwich structure (Lu, C et al. (1998) J Biol Chem 273:15138-15147). The ligand binding site of the I domain, MIDAS, exists as two distinct conformations allosterically regulated by the C-terminal α7-helix.

In one embodiment, the targeting moiety utilized in the present invention preferentially associates or binds to an activated integrin, yet does not significantly associate with or bind to the inactive form of the integrin under physiological conditions. One such embodiment of this is where the targeting agent preferentially associates or binds to the active conformation of the α subunit of the integrin on leukocytes, e.g., LFA-1, MAC-1. In one embodiment, the targeting moiety binds selectively to the LFA-1 I-domain of the α-subunit. Such a targeting moiety can be generated or identified by the skilled practitioner. For instance, such a targeting moiety can be selected for by virtue of its ability to bind preferentially to a molecule which possesses epitopes present on one conformation of the LFA-1 molecule, a locked (high-affinity or low affinity) I domain, over its ability to bind a similar locked opposite conformation (low-affinity or high-affinity, respectively) I domain, stabilized by engineered disulfide bonds (Shimaoka, M. et al., Proc. Natl. Acad. Sci. U.S.A. 98, 6009-6014. (2001)), as demonstrated in Example I below.

The targeting moieties of the present invention which selectively bind to activated leukocytes include antibodies that selectively bind to the active conformation of the integrin molecule, e.g. the open conformation of the I domain. In one embodiment, a targeting moiety for an activation specific epitope binds selectively to the leukocyte integrin I-domain in the open, high-affinity conformation. The open conformation is discussed and antibodies to the open conformation, including methods to obtain such antibodies are disclosed in U.S. Pat. Appl. Nos. 20020123614, 20050260192, 20050182244, and U.S. Ser. No. 60/749,672, incorporated herein by reference in their entirety. In particular, the antibodies and binding proteins disclosed in WO 05/079515 and U.S. Pat. No. 5,877,295 are useful as targeting moieties for the present invention, as are functional fragments and derivatives thereof. In one embodiment, the targeting moiety is the antibody AL-57 (described in WO 05/079515 as D2-57; Huang, et al. Identification and characterization of a human monoclonal antagonistic antibody AL-57 specific for the high affinity form of lymphocyte function-associated antigen-1 (submitted); Shimaoka et al. An engineered monoclonal antibody AL-57 preferentially recognizes the high affinity open conformation of integrin LFA-1 in a ligand-mimetic manner. (in preparation)) or a functional fragment thereof, and the target is the activated form of LFA-1. In another embodiment, the targeting moiety is CBRM 1/5 (described in U.S. Pat. No. 5,877,295) or functional fragment thereof, and the target is MAC-1.

In another embodiment, the targeting moiety for activated leukocytes is an agent which specifically binds the β₂ leg of LFA-1.

An antibody or functional fragment or derivative thereof, which serves as a targeting moiety for a specific activation state of the integrin selectively binds to an epitope that is unique to that activation state of the integrin. Such epitopes may otherwise be buried and not available for binding when the integrin is in one conformation, but become exposed upon adoption of the other conformation. For example, the epitope of KIM127 is buried in the ‘genu’ in the inactive bent conformation, whereas it is exposed in the active extended conformation (Beglova et al., 2002, Nat. Struct. Biol. 9, 282-287; Lu et al., 2001, J. Immunol. 166, 5629-5637). Also see the first figure of Salas et al., (2004, Immunity 20, 393406). Alternatively, such epitopes may not exist in the unrecognized conformation, but be generated by bringing together of the necessary components upon adoption of the recognized conformation. An epitope that is unique to an activated integrin is herein referred to as an activation specific epitope. An epitope that is unique to an inactived integrin is herein referred to as an inactivation specific epitope. Such epitopes are typically found in the regions of an integrin which directly bind ligand, although they will also exist in other regions as well (e.g. regions adjacent to the regions which bind ligand, or regions of the molecule which are not involved in ligand binding, but are otherwise affected by the conformational change which permits ligand binding).

In one embodiment, the targeting moiety specific for activated leukocytes is the monoclonal antibody KIM127 or a functional fragment thereof. KIM127 is an activation-dependent and activating antibody which maps to the I-EGF2 in the β₂ leg. The epitope of KIM127 is buried in the ‘genu’ in the inactive bent conformation, whereas it is exposed in the active extended conformation (Beglova et al., 2002, Nat. Struct. Biol. 9, 282-287; Lu et al., 2001, J. Immunol. 166, 5629-5637). Also see the first figure in Salas et al., (2004, Immunity 20, 393406).

The targeting moiety may also be derived from the ligand or counter-receptor which naturally binds the targeted integrin. The counterreceptors for integrins are ICAMs. The targeting moiety could encompass the complete ligand, or a peptide fragment or derivative thereof (e.g. a modified peptide fragment) which retains integrin binding activity. Examples of such ICAM derived peptides useful for the targeting moiety of the present invention are disclosed in U.S. Pat. No. 5,288,854, U.S. Pat. Appl. No. 20040037775 or WO 05/002516. ICAM peptides may be comprised of naturally occurring peptides or synthetic peptidomimics.

In one embodiment, the targeting moiety specifically binds to the activated integrin conformation (the open conformation) in a ligand-mimetic manner. One example of such a targeting moiety is the monoclonal antibody AL-57, or a functional fragment thereof. In another embodiment, the targeting moiety specifically binds the activated integrin conformation, but in a non-ligand mimetic manner.

Certain targeting moieties in binding to LFA-1, e.g., LFA-1 in active conformation, inhibit binding of LFA-1 to its cognate ligands. To a certain extent, by inhibiting LFA-1 binding, the bound targeting moiety further treats the disease. However, the use of targeting moieties which do not interfere with ligand binding is still expected to provide therapeutic benefit.

In another embodiment, the target cell population is inactive leukocytes. This is accomplished by targeting an epitope of an integrin which is only displayed/available for binding when the integrin is in the inactive conformation (e.g., as expressed on the closed conformation of the I-domain of the α-subunit of LFA-1).

### Carriers for Therapeutic Agents

The carrier particles for the therapeutic agents include any carrier particle modifiable by attachment of a targeting moiety known at the time. Suitable carrier particles include, without limitation, liposomes, proteins, and polymers. Carrier particles may be selected according to their ability to transport the therapeutic agent of choice and the ability to covalently attach the targeting moiety to the carrier particle.

In one preferred embodiment, the carrier particle is a liposome particle, otherwise referred to herein as a liposome. The outer surface of the liposomes may be modified. One example of such a modification is modification of the outer surface of the liposome with a long-circulating agent, e.g., PEG, e.g., hyaluronic acid (HA). The liposomes may be modified with a cryoprotectant, e.g., a sugar, such as trehalose, sucrose, mannose or glucose, e.g., HA. In one preferred embodiment, the liposome is coated with HA. HA acts as both a long-circulating agent and a cryoprotectant. Methods and specific examples for coating a liposome are provided in US Provisional Application titled LAYER BY LAYER COATING OF IMNOLIPOSOMES, 60/794,361, filed Apr. 24, 2006, and in the corresponding PCT application METHOD OF PRODUCING IMMUNOLIPOSOMES AND COMPOSITIONS THEREOF, filed Apr. 24, 2007, both of which contents are incorporated herein by reference. These documents describe methods for coating small lipid particles, e.g., liposomes or micelles, layer-by-layer with a first layer of a cryoprotectant and a second layer of a targeting agent, e.g., antibody, scFv, or a receptor ligand, and the products thus produced. They further describe a method for encapsulating agents in a particle having both a cryoprotectant and a targeter. As such, liposomes comprising multiple layers assembled in a step-wise fashion are suitable for use as a carrier in the present invention.

Such liposomes are prepared from empty nano-scale liposomes prepared by any method known to the skilled artisan from any liposome material known at the time. To this, a first layer of surface modification is added to the liposome by covalent modification. The first layer comprises a cryoprotectant such as hyaluronic acid, or glucosaminoglycan. To this, a second layer of surface modification is added by covalent attachment to the first layer. The second layer may serve as a targeting agent or moiety as described herein, e.g., an antibody or functional fragment thereof. Further layers may add to the liposome additional agents (e.g. additional targeting moieties). Alternatively, the second layer may include a heterogeneous mix of targeting moieties. The liposome composition is lyophilized after addition of the final layer. The therapeutic agent of interest is encapsulated by the liposome by rehydration of the liposome with an aqueous solution containing the agent (e.g. drug). Therapeutic agents that are poorly soluble in aqueous solutions or agents that are hydrophobic may be added to the composition during preparation of the liposomes in step one. The liposome composition is optionally lyophilized and reconstituted at any time after the addition of the first layer.

The term “cryoprotectant” refers to an agent that protects a lipid particle subjected to dehydration-rehydration, freeze-thawing, or lyophilization-rehydration from vesicle fusion and/or leakage of vesicle contents. Useful cryoprotectants in the methods of the present invention include hyaluronan/hyaluronic acid (HA) or other glycosaminoglycans for use with liposomes or micelles or PEG for use with micelles.

The liposome preparation of the present invention is characterized in that it is further derivatized with a cryoprotectant. One preferred cryoprotectant of the present invention is hyaluronic acid or hyaluran (HA). Hyaluronic acid, a type of glycosaminoglycan, is a natural polymer with alternating units of N-acetyl glucosamine and glucoronic acid. Using a crosslinking reagent, hyaluronic acid offers carboxylic acid residues as functional groups for covalent binding. The N-acetyl-glucosamine contains hydroxyl units of the type —CH₂—OH which can be oxidized to aldehydes, thereby offering an additional method of crosslinking hyaluronic acid to the liposomal surface in the absence of a crosslinking reagent. Alternatively, other glycosaminoglycans, e.g., chondroitin sulfate, dermatan sulfate, keratin sulfate, or heparin, may be utilized in the methods of the present invention. Cryoprotectants are bound covalently to discrete sites on the liposome surfaces. The number and surface density of these sites will be dictated by the liposome formulation and the liposome type. The final ratio of cryoprotectant (μg) to lipid (μmole) is about 50 μg/μmole, about 55 μg/μmole, about 60 μg/μmole, about 65 μg/μmole, about 70 μg/μmole, about 75 μg/μmole, about 80 μg/μmole, about 85 μg/μmole, about 90 μg/μmole, about 95 μg/μmole, about 100 μg/μmole, about 105 μg/μmole, about 120 μg/mole.

Crosslinking reagents can be used to form covalent conjugates of cryoprotectants and liposomes. Such crosslinking reagents include glutaraldehyde (GAD), bifunctional oxirane (OXR), ethylene glycol diglycidyl ether (EGDE), and a water soluble carbodiimide, preferably 1-ethyl-3-(3-dimethylaminopropyl)carbodiimide (EDC). Through the complex chemistry of crosslinking, linkage of the amine residues of the recognizing substance and liposomes is established. Covalent attachment of the cryoprotectant HA is described in U.S. Pat. No. 5,846,561.

The outer surface of the liposomes may be further modified with a long-circulating agent in order to prevent the uptake of the liposomes into the cellular endothelial systems and enhance the uptake of the liposomes into the tissue of interest. The modification of the liposomes with a hydrophilic polymer as the long-circulating agent is known to enable to prolong the half-life of the liposomes in the blood. Examples of hydrophilic polymer suitable for use include polyethylene glycol, polymethylethylene glycol, polyhydroxypropylene glycol, polypropylene glycol, polymethylpropylene glycol and polyhydroxypropylene oxide. Glycosaminoglycans, e.g., hyaluronic acid, may also be used as long-circulating agents.

The liposome is modified by attachment of the targeting moiety. In one embodiment, the targeting moiety is covalently conjugated to the cryoprotectant, e.g., HA. This can be accomplished using a crosslinking reagent (e.g. glutaraldehyde (GAD), bifunctional oxirane (OXR), ethylene glycol diglycidyl ether (EGDE), N-hydroxysuccinimide (NHS), and a water soluble carbodiimide, preferably 1-ethyl-3-(3-dimethylaminopropyl)carbodiimide (EDC). As is known to the skilled artisan, any crosslinking chemistry can be used, including, but not limited to, thioether, thioester, malimide and thiol, amine-carboxyl, amine-amine, and others listed in organic chemistry manuals, such as, Elements of Organic Chemistry, Isaak and Henry Zimmerman Macmillan Publishing Co., Inc. 866 Third Avenue, New York, N.Y. 10022. Through the complex chemistry of crosslinking, linkage of the amine residues of the recognizing substance and liposomes is established.

In one embodiment, the targeting moiety is covalently attached to HA, which is bound to the liposome surface. Alternatively, the carrier particle is a micelle. Alternatively, the micelle is modified with a cryoprotectant, e.g., HA, PEG.

The liposome may be unilamellar or multilamellar. In one embodiment the liposome is unilamellar, and the first layer comprises glycoasminoglycan hyaluronan (HA). The HA may optionally be covalently linked to aphosphatidylethanolamine. The unilamellar liposome may further comprise a second layer which has specific antibodies covalently attached to the HA of the first layer.

The therapeutic agent is encapsulated within the liposome carrier. The terms “encapsulation” and “entrapped,” as used interchangeably herein, refer to the incorporation of an agent in a lipid particle. In one embodiment, the agent is encapsulated such that it is present in the aqueous interior of the lipid particle. In one embodiment, a portion of the encapsulated agent takes the form of a precipitated salt in the interior of the liposome. The agent may also self precipitate in the interior of the liposome.

Nucleic acids have a charged backbone that prevents efficient encapsulation in the lipid particle, but can be condensed with a cationic polymer to enhance encapsulation. Accordingly, the nucleic acid therapeutic agent of interest may be condensed with a cationic polymer, e.g., PEI, polyamine spermidine, and spermine, or cationic peptide, e.g., protamine and polylysine, prior to encapsulation in the lipid particle. In one embodiment, the agent is not condensed with a cationic polymer.

In one embodiment, the multi-layered liposomes of the invention is made with cryoprotectant conjugated lipid particles. The cryoprotectant is convalently linked to the lipid polar groups of the phospholipids and it forms the first layer of surface modification on the liposome discussed supra. The targeting agent forms the second layer of coat and it is added on to the first layer of cryoprotectant. The multi-layered liposome may be lyophilized for storage. The agent of interest is encapsulated by the liposome by rehydration of the liposome with an aqueous solution containing the agent.

Other possible cryoprotectants are disaccharide and monosaccharide sugars such as trehalose, maltose, sucrose, maltose, fructose, glucose, lactose, saccharose, galactose, mannose, xylit and sorbit, mannitol, dextran; polyols such as glycerol, glycerin, polyglycerin, ethylene glycol, prolylene glycol, polyethyleneglycol and branched polymers thereof; aminoglycosides; and dimethylsulfoxide.

In one embodiment, the prior to coating, lipid particle is pre-conjugated with a cryoprotectant, wherein the cryoprotectant has a functional group attached. The attached functional group may be activated and a targeting agent is crosslinked to the activated functional group to form a two-layer coated lipid particle which can then be lyophilized for storage purposes prior to use for drug or agent encapsulation.

In one embodiment, two agents of interest (e.g. therapeutic agents) may be delivered by the lipid particle. One agent can be hydrophobic and the other is hydrophilic. The hydrophobic agent may be added to the lipid particle during formation of the lipid particle. The hydrophobic agent associates with the lipid portion of the lipid particle. The hydrophilic agent is added in the aqueous solution rehydrating the lyophilized lipid particle. An exemplary embodiment of two agent delivery is described below, wherein a condensed siRNA is encapsulated in a liposome and wherein a drug that is poorly soluble in aqueous solution is associated with the lipid portion of the lipid particle. As used herein, “poorly soluble in aqueous solution” refers to a composition that is less that 10% soluble in water.

In one embodiment, the carrier moiety is a protein (e.g. a basic polypeptide) or the nucleic acid binding domain of a protein. In one preferred embodiment, the binding moiety is the nucleic acid binding domain of a protein selected from the group of nucleic acid binding domains present in proteins selected from the group consisting of protamine, GCN4, Fos, Jun, TFIIS, FMRI, yeast protein HX, Vigillin, Mer1, bacterial polynucleotide phosphorylase, ribosomal protein S3, and heat shock protein. In one preferred embodiment, the binding moiety is the protein protamine or an RNA interference-inducing molecule-binding fragment of protamine.

When the therapeutic agent is a nucleic acid, a suitable carrier is any agent which complexes with a nucleic acid (e.g. an siRNA). Suitable complexing agents include poly-amino acids; polyimines; polyacrylates; polyalkylacrylates, polyoxethanes, polyalkylcyanoacrylates; cationized gelatins, albumins, starches, acrylates, polyethyleneglycols (PEG) and starches; polyalkylcyanoacrylates; DEAE-derivatized polyimines, pollulans, celluloses and starches. Particularly preferred complexing agents include chitosan, N-trimethylchitosan, poly-L-lysine, polyhistidine, polyornithine, polyspermines, protamine, polyvinylpyridine, polythiodiethylaminomethylethylene P(TDAE), polyaminostyrene (e.g. p-amino), poly(methylcyanoacrylate), poly(ethylcyanoacrylate), poly(butylcyanoacrylate), poly(isobutylcyanoacrylate), poly(isohexylcynaoacrylate), DEAE-methacrylate, DEAE-hexylacrylate, DEAE-acrylamide, DE AE-albumin and DEAE-dextran, polymethylacrylate, polyhexylacrylate, poly(D,L-lactic acid), poly(DL-lactic-co-glycolic acid (PLGA), alginate, and polyethyleneglycol (PEG), and polyethylenimine. In one embodiment, the carrier moiety is selected from the nucleic acid binding domains present in proteins selected from the group consisting of GCN4, Fos, Jun, TFIIS, FMRI, yeast protein HX, Vigillin, Mer1, bacterial polynucleotide phosphorylase, ribosomal protein S3, and heat shock protein.

In one preferred embodiment, the carrier particle is a cationic peptide, e.g. a polycationic peptide such as protamine or a fragment thereof which is functional as a carrier fragment for a nucleic acid, herein referred to as a functional fragment of protamine. WO 06/023491 and WO 06/23491 describes the synthesis and use of such carrier cationic peptides. Protamine is a polycationic peptide which nucleates DNA in sperm. Its nucleic acid binding properties make it useful as a nucleic acid delivery agent. In one embodiment, protamine, or a functional fragment thereof is used to deliver siRNAs via an antibody Fab fragment-protanine fusion protein.

An example of a functional fragment of protamine is nucleic acid binding fragment (e.g. an siRNA-binding fragment) of protamine. Protamine has a molecular weight about 4000-4500 Da. Protamine is a small basic nucleic acid binding protein, which serves to condense the animal's genomic DNA for packaging into the restrictive volume of a sperm head (Warrant, R. W., et al., Nature 271:130-135 (1978); Krawetz, S. A., et al., Genomics 5:639-645 (1989)). The positive charges of the protamine can strongly interact with negative charges of the phosphate backbone of nucleic acid, such as RNA resulting in a neutral and, as shown here, stable interference RNA protamine complex. The methods, reagents and references that describe a preparation of a nucleic acid-protamine complex in detail are disclosed in the U.S. Patent Application Publication Nos. US2002/0132990 and US2004/0023902, and are herein incorporated by reference in their entirety.

In one embodiment, the protamine fragment useful according to the present invention is encoded by a nucleic acid sequence SEQ ID NO: 1, or a homolog thereof capable of encoding the same amino acids as the SEQ ID NO: 1:

(SEQ ID NO: 1)
GCGGCCGCACGCAGCCAGAGCCGGAGCAGATATTACCGCCAGAGACAAAG
AAGTCGCAGACGAAGGAGGCGGAGCTGCCAGACACGGAGGAGAGCCAATG
AGTCTCATCATCACCACCACCATTAA.

In one embodiment, the protamine fragment useful according to the present invention is encoded by a nucleic acid sequence SEQ ID NO: 2, or a homolog therefore capable of encoding the same amino acids as the SEQ ID NO: 2:

(SEQ ID NO: 2)
GCGGCCGCAATGGCCAGGTACAGATGCTGTCGCAGCCAGAGCCGGAGCAG
ATATTACCGCCAGAGACAAAGAAGTCGCAGACGAAGGAGGCGGAGCTGCC
AGACACGGAGGAGAGCCATGAGATCTCATCATCACCACCACCATTAA.

In one embodiment, the protamine fragment useful according to the present invention is encoded by a nucleic acid sequence SEQ ID NO: 3, or a homolog therefore capable of encoding the same amino acids as the SEQ ID NO: 3:

(SEQ ID NO: 3)
GCGGCCGCACGCAGCCAGAGCGGGAGCAGATATTACCGCCAGAGACAAAG
AAGTCGCAGACGAAGGAGGCGGAGCTGCCAGACACGGAGGAGAGCCATGA
GGTGTTGTCGCCCCAGGTACAGACCGAGATGTAGAAGACACAGATCTCAT
CATCACCACCACCATTAA

In one embodiment, the protamine fragment useful according to the present invention is encoded by a nucleic acid sequence SEQ ID NO: 4, or a homolog therefore capable of encoding the same amino acids as the SEQ ID NO: 4:

(SEQ ID NO: 4)
GCGGCCGCACGCAGCCAGAGCCGGAGCAGATATTACCGCCAGAGACAA
AGAAGTCGCAGACGAAGGAGGCGGAGCAGATCTCATCATCACCACCAC
CATTAA

In one embodiment, the protamine fragment useful according to the present invention is encoded by a nucleic acid sequence SEQ ID NO: 5, or a homolog therefore capable of encoding the same amino acids as the SEQ ID NO: 5:

(SEQ ID NO: 5)
GCGGCCGCCGGCGGAGGAGGATCTGATCATCACCAGCATTAA

In one embodiment, the protamine fragment useful according to the present invention is encoded by a nucleic acid sequence SEQ ID NO: 6, or a homolog therefore capable of encoding the same amino acids as the SEQ ID NO: 6:

(SEQ ID NO: 6)
GCGGCCGCAATGGCCAGGTACAGATGCTGTCGCAGCCAGAGCCGGAGGA
GATATTACCGCCAGAGACAAAGAAGTCGCAGACGAAGGAGGCGGAGCAG
ATCTCATCATCACCACCACCATTAA.

In one embodiment, the protamine fragment has the amino acid sequence

(SEQ ID NO: 7)
RSQSRSRYYRQRQRSRRRRRRS.

In one embodiment, the carrier is full length protamine.

The protein carrier moiety may also include additional amino acid sequences, or other modifications (e.g glycosylation) which confer one or more desired properties. Relatedly, it may also be useful to make a chimeric protein carrier moiety, generated from different sources, e.g. a combination of one or more fragments of a protein carrier moiety described herein.

The glycosaminoglycan carrier particles disclosed in U.S. Pat. Appl. No. 20040241248 and the glycoprotein carrier particles in WO 06/017195 may be used in the methods and compositions of the present invention.

Soluble polymers are also useful as carrier particles. Such polymers can include polyvinylpyrrolidone, pyran copolymer, polyhydroxypropylmethacrylamidephenol, polyhydroxyethylaspartamidephenol, or polyethyleneoxidepolylysine substituted with palitoyl residues. Furthermore, the compounds may be coupled to a class of biodegradable polymers useful in achieving controlled release of a drug, for example, polylactic acid, polepsilon caprolactone, polyhydroxy butyric acid, polyorthoesters, polyacetals, polydihydropyrans, polycyanoacrylates, and cross-linked or amphipathic block copolymers of hydrogels. The substances can also be affixed to rigid polymers and other structures such as fullerenes or Buckeyballs.

In one embodiment, the carrier particle is not a polymer. In one embodiment, the carrier particle is not a protein, e.g., cationic peptide, glycoprotein.

The targeting moiety may be associated with the protein carrier moiety by a covalent (e.g. by fusion, chemical cross linking or conjugation) or non-covalent association (e.g. through binding of a specific binding pair). The location of the association of the targeting moiety on the carrier moiety may be anywhere which does not interfere with the necessary activities of either moiety. (e.g, the carboxyl-terminal or amino-terminal end or in the middle). The delivery agent may also comprise more than one carrier moieties (with one or more therapeutic agents) and one or more targeting moieties.

Covalent attachment further includes the embodiment wherein the carrier particle is a protein, e.g., a protamine, and the targeting moiety is a protein, e.g., an antibody or functional fragment thereof, e.g., a peptide such as an ICAM peptide, and the carrier particle and the targeting moiety comprise a fusion protein.

### Therapeutic Agents

The compositions and methods of the present invention are useful for the treatment or diagnosis of diseases which arise from or otherwise involve leukocyte action or inaction. One such type of disease is a disease associated with inappropriately activated leukocytes. A therapeutic agent as the term is used herein, is an agent, which when delivered to a target cell, effects the target cell in such a way as to contribute to treatment of a disease in the recipient subject. As used herein, the terms “treating” or “treatment” of a disease include preventing the disease, i.e. preventing clinical symptoms of the disease in a subject that may be exposed to, or predisposed to, the disease, but does not yet experience or display symptoms of the disease; inhibiting the disease, i.e., arresting the development of the disease or its clinical symptoms; or relieving the disease, i.e., causing regression of the disease or its clinical symptoms. A therapeutic agent may also be an agent useful for diagnosis of disease or disease progression or of effects of treatment of the disease.

In one embodiment, a subject who receives an administered delivery agent of the present invention exhibits inappropriate leukocyte activation prior to administration of the delivery agent.

Useful therapeutic agents include nucleic acids, small molecules, polypeptides, antibodies or functional fragments thereof. These core components as therapeutic agents may be further by modified to enhance function or storage, (e.g. enhance cellular uptake, increase specificity for the target, increase half-life, facilitate generation or storage). Nucleic acid therapeutic agents include DNA and RNA molecules, both doubles stranded and single stranded. More than one therapeutic agent may be delivered by the delivery agent of the present invention.

Therapeutic agents delivered by the methods of the present invention include agents which target proinflammatory mediators such as cytokine and chemokine genes, enzymes involved in generation of inflammatory mediators, receptors for cytokines, chemokines, lipid mediators, apoptosis, cytoplasmic signaling molecules involved in inflammatory cascades, e.g., NF-kB, STAT, Talin, Rap-1; tissue injury such as apoptosis, e.g., caspase, bcl-2; molecules important for cell activation and proliferation, e.g., cyclins, kinesin Eg5; molecules important for cell movement/migration/invasion, e.g., small G-proteins, cytoskeletat proteins; and oncogenes. Specific targeting of CD4 may be used for blocking HIV infection. Specific targeting of Ku70 may be used for killing or suppressing cancer cells. Specific targeting Cyclin-D1 may be used for blocking proliferation.

In addition, delivery agents comprising therapeutic agents which have therapeutics for treating diseases such as viral diseases are included in the present invention. One example of such a delivery agent is an siRNA which serves as a microbicides. This is useful for treatment and/or prevention of HSV, HPB and HIV. Such therapeutic agents are described in PCT/US2006/021758 and PCT/US2003/034424, the contents of which are herein incorporated by reference in their entirety.

Therapeutic agents delivered by the methods of the present invention include small molecules chemicals and peptides to block intracellular signaling cascades, enzymes (kinases), proteosome, lipid metabolism, cell cycle, membrane trafficking. Therapeutic agents delivered by the methods of the present invention include chemotherapy agents.

The therapeutic agents may be associated with the carrier particle (e.g. liposome or protamine) by any method known to the skilled artisan. In embodiments where the carrier particle is a liposome this includes, without limitation, encapsulation in the interior, association with the lipid portion of the molecule or association with the exterior of the liposome. Small molecule drugs soluble in aqueous solution may be encapsulated in the interior of the liposome. Small molecule drugs that are poor soluble in aqueous solution may associate with the lipid portion of the liposome. Nucleic acid based therapeutic agents may associate with the exterior of the liposome. Such nucleic acids may be condensed with cationic polymers, e.g., PEI, or cationic peptides, e.g., protamines, and encapsulated in the interior of the liposome. Therapeutic peptides may be encapsulated in the interior of the liposome. Therapeutic peptides may be covalently attached to the exterior of the liposome.

In embodiments where the therapeutic agent is a nucleic acid, it may be particularly useful to have a carrier moiety which is particularly suitable for nucleic acid transport.

In one embodiment, the therapeutic agent is a nucleic acid, such as an RNA or DNA molecule (e.g. a double stranded or single stranded DNA oligonucleotide). Useful DNA molecules are antisense as well as sense (e.g. coding and/or regulatory) DNA. Antisense DNA molecules include short oligonucleotides. Useful RNA molecules include RNA interference molecules, of which there are several known types. The field of RNA interference molecules has greatly expanded in recent years. Examples of RNA interference molecules useful in the present invention are siRNA, dsRNA, stRNA, shRNA, and miRNA (e.g., short temporal RNAs and small modulatory RNAs (Kim. 2005. Mol Cells. 19:1-15)). As used herein, “double stranded RNA” or “dsRNA” refers to RNA molecules that are comprised of two strands. Double-stranded molecules include those comprised of a single RNA molecule that doubles back on itself to form a two-stranded structure. For example, the stem loop structure of the progenitor molecules from which the single-stranded miRNA is derived, called the pre-miRNA (Bartel et al. 2004. Cell 116:281-297), comprises a dsRNA molecule. Other RNA molecules which are single stranded, or are not considerd to be RNA inhibition molecules may also be useful as therapeutic agents, including messenger RNAs (and the progenitor pre-messenger RNAs), small nuclear RNAs, small nucleolar RNAs, transfer RNAs and ribosomal RNAs.

Numerous specific siRNA molecules have been designed that have been shown to inhibit gene expression (Ratcliff et al. Science 276:1558-1560, 1997; Waterhouse et al. Nature 411:834-842, 2001). In addition, specific siRNA molecules have been shown to inhibit, for example, HIV-1 entry to a cell by targeting the host CD4 protein expression in target cells thereby reducing the entry sites for HIV-1 which targets cells expressing CD4 (Novina et al. Nature Medicine, 8:681-686, 2002). Short interfering RNA have further been designed and successfully used to silence expression of Fas to reduce Fas-mediated apoptosis in vivo (Song et al. Nature Medicine 9:347-351, 2003). Accordingly, the RNA interference-inducing molecule referred to in the specification includes, but is not limited to, unmodified and modified double stranded (ds) RNA molecules including, short-temporal RNA (stRNA), small interfering RNA (siRNA), short-hairpin RNA (shRNA), microRNA (miRNA), double-stranded RNA (dsRNA), (see, e.g. Baulcombe, Science 297:2002-2003, 2002). The dsRNA molecules, e.g. siRNA, also may contain 3′ overhangs, preferably 3′UU or 3′TT overhangs. In one embodiment, the siRNA molecules of the present invention do not include RNA molecules that comprise ssRNA greater than about 3040 bases, about 40-50 bases, about 50 bases or more. In one embodiment, the siRNA molecules of the present invention have a double stranded structure. In one embodiment, the siRNA molecules of the present invention are double stranded for more than about 25%, more than about 50%, more than about 60%, more than about 70%, more than about 80%, more than about 90% of their length.

As used herein, “gene silencing” induced by RNA interference refers to a decrease in the mRNA level in a cell for a target gene by at least about 5%, about 10%, about 20%, about 30%, about 40%, about 50%, about 60%, about 70%, about 80%, about 90%, about 95%, about 99%, about 100% of the mRNA level found in the cell without introduction of RNA interference. In one preferred embodiment, the mRNA levels are decreased by at least about 70%, about 80%, about 90%, about 95%, about 99%, about 100%.

The RNA interference as described herein also includes RNA molecules having one or more non-natural nucleotides, i.e. nucleotides other than adenine “A”, guanine “G”, uracil “U”, or cytosine “C”, a modified nucleotide residue or a derivative or analog of a natural nucleotide are also useful. Any modified residue, derivative or analog may be used to the extent that it does not eliminate or substantially reduce (by at least 50%) RNAi activity of the dsRNA. These forms thus include, but are not limited to, aminoallyl UTP, pseudo-UTP, 5-I-UTP, 5-I-CTP, 5-Br-UTP, alpha-S ATP, alpha-S CTP, alpha-S GTP, alpha-S UTP, 4-thio UTP, 2-thio-CTP, 2′NH₂ UTP, 2′NH₂ CTP, and 2′F. UTP. Such modified nucleotides include, but are not limited to, aminoallyl uridine, pseudo-uridine, 5-I-uridine, 5-I-cytidine, 5-Br-uridine, alpha-S adenosine, alpha-S cytidine, alpha-S guanosine, alpha-S uridine, 4-thio uridine, 2-thio-cytidine, 2′NH₂ uridine, 2′NH₂ cytidine, and 2′F uridine, including the free pho (NTP) RNA molecules as well as all other useful forms of the nucleotides.

The RNA interference as referred herein additionally includes RNA molecules which contain modifications in the ribose sugars, as well as modifications in the “phosphate backbone” of the nucleotide chain. For example, siRNA or miRNA molecules containing α-D-arabinofuranosyl structures in place of the naturally-occurring α-D-ribonucleosides found in RNA can be used in RNA interference according to the present invention (U.S. Pat. No. 5,177,196). Other examples include RNA molecules containing the o-linkage between the sugar and the heterocyclic base of the nucleoside, which confers nuclease resistance and tight complementary strand binding to the oligonucleotidesmolecules similar to the oligonucleotides containing 2′-O-methyl ribose, arabinose and particularly α-arabinose (U.S. Pat. No. 5,177,196). Also, phosphorothioate linkages can be used to stabilize the siRNA and miRNA molecules (U.S. Pat. No. 5,177,196). siRNA and miRNA molecules having various “tails” covalently attached to either their 3′- or to their 5′-ends, or to both, are also been known in the art and can be used to stabilize the siRNA and miRNA molecules delivered using the methods of the present invention. Generally speaking, intercalating groups, various kinds of reporter groups and lipophilic groups attached to the 3′ or 5′ ends of the RNA molecules are well known to one skilled in the art and are useful according to the methods of the present invention. Descriptions of syntheses of 3′-cholesterol or 3′-acridine modified oligonucleotides applicable to preparation of modified RNA molecules useful according to the present invention can be found, for example, in the articles: Gamper, H. B., Reed, M. W., Cox, T., Virosco, J. S., Adams, A. D., Gall, A., Scholler, J. K., and Meyer, R. B. (1993) Facile Preparation and Exonuclease Stability of 3′-Modified Oligodeoxynucleotides. Nucleic Acids Res. 21 145-150; and Reed, M. W., Adams, A. D., Nelson, J. S., and Meyer, R. B., Jr. (1991) Acridine and Cholesterol-Derivatized Solid Supports for Improved Synthesis of 3′-Modified Oligonucleotides. Bioconjugate Chem. 2 217-225 (1993).

Various specific siRNA and miRNA molecules have been described and additional molecules can be easily designed by one skilled in the art. (Griffiths-Jones S, NAR, 2004, 32, Database Issue, D109-D111; Ambros V, Bartel B, Bartel D P, Burge C B, Carrington J C, Chen X, Dreyfuss G, Eddy S R, Griffiths-Jones S, Marshall M, Matzke M, Ruvkun G, Tuschl T. RNA, 2003, 9(3), 277-279). An “siRNA” as used herein and throughout the specification refers to a nucleic acid that forms a double stranded RNA, which double stranded RNA has the ability to reduce or inhibit expression of a gene or target gene when the siRNA is expressed in the same cell as the gene or target gene. “siRNA” thus refers to the double stranded RNA formed by the complementary strands. The complementary portions of the siRNA that hybridize to form the double stranded molecule typically have substantial or complete identity. In one embodiment, an siRNA refers to a nucleic acid that has substantial or complete identity to a target gene and forms a double stranded siRNA. The sequence of the siRNA can correspond to the full length target gene, or a subsequence thereof. Typically, the siRNA is at least about 15-50 nucleotides in length (e.g., each complementary sequence of the double stranded siRNA is about 15-50 nucleotides in length, and the double stranded siRNA is about 15-50 base pairs in length, preferably about 19-30 base nucleotides, preferably about 20-25 nucleotides in length, e.g., 20, 21, 22, 23, 24, 25, 26, 27, 28, 29, or 30 nucleotides in length).

siRNAs also include small hairpin (also called stem loop) RNAs (shRNAs). In one embodiment, these shRNAs are composed of a short, e.g. about 19 to about 25 nucleotide, antisense strand, followed by a nucleotide loop of about 5 to about 9 nucleotides, and the analogous sense strand. Alternatively, the sense strand may precede the nucleotide loop structure and the antisense strand may follow.

Useful siRNA molecules as therapeutic agents of the present invention include, without limitation, CCR5-siRNA, ku70-siRNA, CD4-siRNA or cyclin-D1-siRNA. The present invention also includes combinations of therapeutics agents.

Another nucleic acid based therapeutic agent of the present invention is an antagomir (Krutzfeldt et al., Nature vol. 438, no. 7068, pp. 685-689). An antagomir is a chemically modified, cholesterol-conjugated single-stranded RNA analogue complementary to an miRNA, used to inhibit or silence an miRNA in vivo.

Another therapeutic agent of the present invention is a locked nucleic acid (LNA), sometimes referred to as an inaccessible RNA. An LNA is a modified RNA nucleotide wherein the ribose moiety of LNA nucleotide is modified with an extra bridge connecting 2′ and 4′ carbons. This enhances the base stacking and pre-organization, and significantly increases the thermal stability. This bridge “locks” the ribose in 3′-endo structural conformation, which is often found in A-form of DNA or RNA. LNA nucleotides used in the present invention can be mixed with DNA or RNA bases in the oligonucleotide whenever desired. Such oligomers are commercially available.

Protamine carrier particles are particularly useful for transporting nucleic acids such as those described herein. The cationic arginine rich peptide 11 dR can be used as well (Melikov et al., Cell Mol Life Sci. 2005; 62: 2739-49) may be used.

The therapeutic agent may be an antagonist of LFA-1 and/or MAC-1. For example, U.S. Pat. Appl. No. 20050203135 discloses LFA-1 and MAC-1 antagonists and U.S. Pat. No. 6,667,318 discloses LFA-1 antagonists.

The therapeutic agent may be encapsulated along with a pharmaceutically acceptable carrier.

### Methods

The methods of the present invention are useful for delivering an agent to a target cell or a population of target cells using the delivery agent described herein. The target cell(s) can be isolated or can exist within a mixed population of cells (containing non-target cells). The target cell(s) can be within the body of an individual, or can be in vitro (e.g. grown in cell culture, isolated from an individual). The target cell(s) can be isolated from an individual for treatment, including contact with the delivery agent, and then re-administered to the individual following the desired treatment (ex vivo). As such, the delivery agent of the present invention can be used for in vivo delivery, in vitro delivery and ex vivo delivery. The in vivo delivery as used herein means delivery of the delivery agent of the present invention into a living subject, including human. The in vitro delivery as used herein means delivery of the delivery agent into cells and organs which are removed from/outside a living subject. Ex vivo delivery is a term which is used to refer to obtaining tissue, cells or organ from a living subject, subjecting it to delivery outside of the body, and then reintroducing the tissue, cell(s) or organ back into the same living subject.

The targeting moiety is contacted to the target cell(s) preferably under physiological conditions, to preserve the integrity of the cells, and to promote effective association (e.g. binding) of the targeting moiety to the integrin receptor and where appropriate, effective uptake of the therapeutic agent by the cell. The cells may be in a mixed population of cells, e.g. in the body of an individual, or removed from the body of an individual). One example of a mixed population of cells removed from the body of an individual would be cells obtained from the blood or secretions of an individual, or from a tumor biopsy of an individual.

The route of delivery (administration) of the delivery agent to a subject relates directly to the particular target cell and to the particular disorder being treated or prevented. This can be determined by the skilled practitioner. Examples of different routes of delivery are intravenous (I.V.), intramuscular (I.M.), subcutaneous (S.C.), intradermal (I.D.), intraperitoneal (I.P.), intrathecal (I.T.), intrapleural, intrauterine, rectal, vaginal, topical, intratumor and the like.

The subject may be any animal for which therapy/delivery is desired. This includes a mouse, rat, high primate, low primate, rabbit, guinea pig, dog, cat, farm animals such as cows, horses, pigs, sheep,).

The target cells may further be from an animal involved in scientific research.

Another aspect of the present invention relates to methods for screening targets of pharmaceutical intervention comprising the steps of delivering a plurality of different therapeutic agents via the deliver agents described herein into cells in parallel cell culture environments, and measuring the effects of targeted genes (e.g. silencing, enhancing). The measurement of effects can be performed either by detecting target RNA molecules using traditional Northern blot analysis or more quantitative methods such as RT-PCR-based RNA quantification or other RNA quantification methods well known to one skilled in the art. Alternatively, silencing or enhancing expression of targeted genes can be detected using traditional immunohistochemical methods to determine presence and/or absence of the protein produced by the target. Detection of a significant desired effect on a target is an indication that the targeting moiety is useful for pharmaceutical intervention.

In one respect, the present invention relates to the herein described compositions, methods, and respective component(s) thereof, as essential to the invention, yet open to the inclusion of unspecified elements, essential or not. In some embodiments, other elements to be included in the description of the composition, method or respective component thereof are limited to those that do not materially affect the basic and novel characteristic(s)” of the invention. This applies equally to steps within a described method as well as compositions and components therein. In other embodiments, the inventions, compositions, methods, and respective components thereof, described herein are intended to be exclusive of any element not deemed an essential element to the component, composition or method.

Unless otherwise defined herein, scientific and technical terms used in connection with the present application shall have the meanings that are commonly understood by those of ordinary skill in the art. Further, unless otherwise required by context, singular terms shall include pluralities and plural terms shall include the singular.

Other than in the operating examples, or where otherwise indicated, all numbers expressing quantities of ingredients or reaction conditions used herein should be understood as modified in all instances by the term “about.” The term “about” when used in connection with percentages may mean 1%.

It should be understood that this invention is not limited to the particular methodology, protocols, and reagents, etc., described herein and as such may vary. The terminology used herein is for the purpose of describing particular embodiments only, and is not intended to limit the scope of the present invention, which is defined solely by the claims.

All patents, patent applications, and publications identified are expressly incorporated herein by reference for the purpose of describing and disclosing, for example, the methodologies described in such publications that might be used in connection with the present invention. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

### Example 1

### Targeted Delivery of siRNA to Activated Leukocytes via Antibody Selective to High-Affinity Form of Integrin LFA-1

Development and characterization of AL-57.

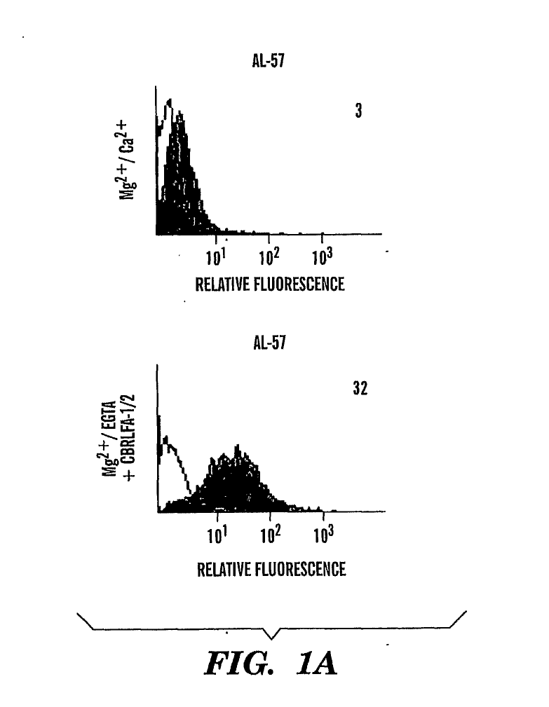

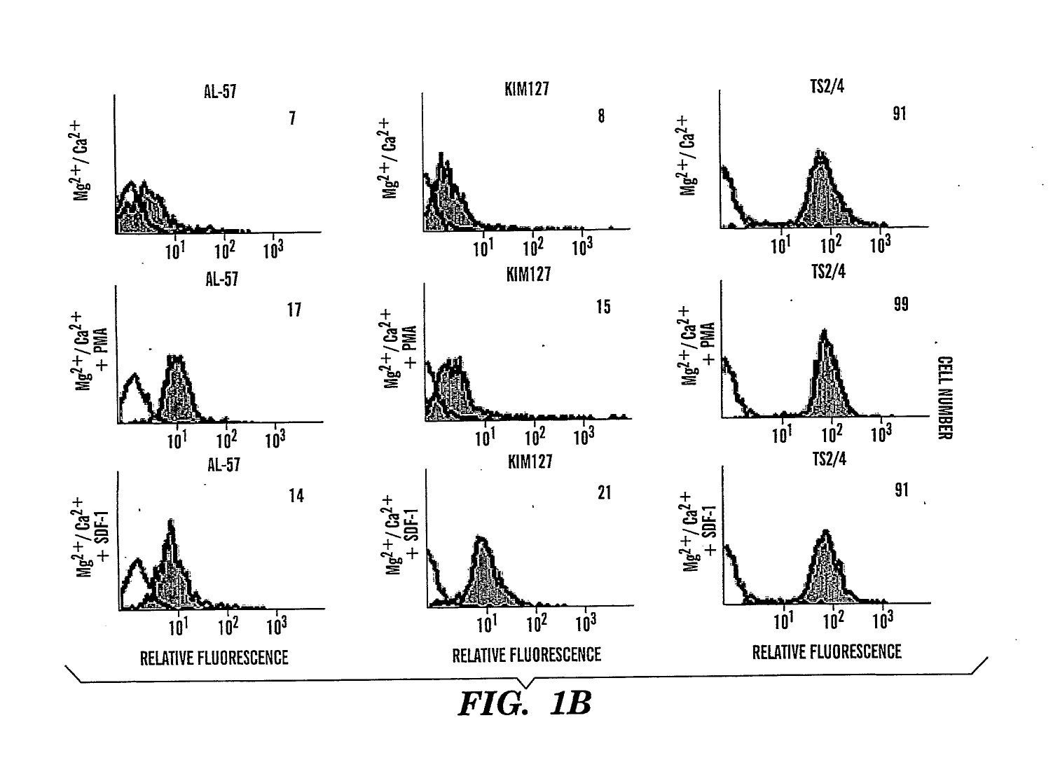

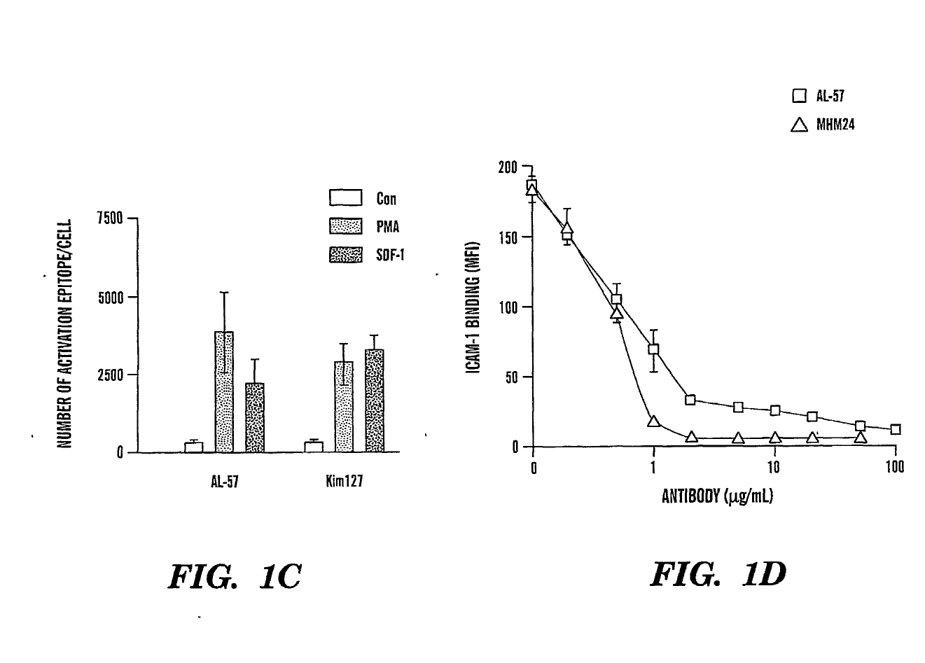

We developed soluble designer LFA-1 I domains that were stabilized by engineered disulfides either in the high-affinity or low-affinity conformation [74]. We used the locked high-affinity I domain (K287C/K294C) and the locked low-affinity I domain (L289C/K294C) for selecting a phage library. A large human Fab library containing 3.7×10¹⁰ different Fab clones [75] was positively selected with the locked high-affinity I domain, and negatively selected with locked low-affinity I domain. After three rounds of selection, individual clones were examined by phage-ELISA. Clones that bound to locked open I domain better than locked closed I domain were selected and further investigated. Finally, one clone (#57) was identified that bound only to the open I domain in a cation-dependent manner and was termed AL-57 (Active LFA-1 clone 57). Fab was converted to an intact IgG. AL-57 was shown to bind to LFA-1 on the cell surface only upon activation with Mg/EGTA plus activating antibody CBRLFA-1/2 (FIG. 1A), and a chemokine CXCL12 (SDF-1) (FIGS. 1B & 1C). In addition, AL-57 inhibited LFA-1-ICAM-1 interaction (FIG. 1D).

Preparation of Immuno-Nanoparticles.

We have developed novel unilamellar liposomes that contain two modified layers on their surface. The 1st layer comprises a glycosaminoglycan hyaluronan (HA) that is covalently linked to phosphatidylethanolamine of the lipid layer. The 2nd layer contains specific antibodies covalently attached to HA of the 1st layer. HA acts bi-functionally as strong cryoprotectant and potent long-circulating agent [47, 76]. The general stabilizing effects of HA that serve in cryoprotection also protect the liposomes during the lyophilization and re-hydration steps required for siRNA encapsulation. In the absence of HA coating, upon lyophilization/re-hydration unilamellar nano-liposomes were unable to maintain their structural and functional integrity: their size was significantly increased and the binding capacity of surface attached antibodies was lost, and became unsuitable for in vivo application.

Lipids were from Avanti Polar lipids, Inc. Regular multilamellar liposomes (MLL) composed of phosphatidylcholine: phosphatidylethanolamine: cholesterol at mole ratios of 3:1:1, were prepared by the traditional lipid-film method as described [47, 77, 80, 81]. Unilamellar nano-scale liposomes (ULNL) were obtained by extrusion of the MLL. The surface modification of the first layer of ULNL with HA was performed as described [47, 78]. The final ratio of HA to lipid was 57 μg HA/Pmole lipid. In the second layer, we covalently attached one of two LFA-1 antibodies or corresponding isotype control antibodies as follows;

AL-57, activation-dependent mAb that preferentially binds to the high affinity LFA-1 I domain (human IgG1)

TS1/22, activation-independent conventional antibody to LFA-1 I domain (mouse IgG1)

Immuno-nanoparticles were purified by gel filtration using a Sephadex G-75 column. Lyophilization of liposome suspensions was performed on 1.0 ml aliquots. Samples were frozen for 2-4 hours at −800° C. and lyophilized for 48 hrs. All procedures were done aseptically.

We studied the size distribution and the surface charge (Zeta potential at pH7.4) of the modified liposomes using a Zetasizer nano SZ™ instrument (Malvern, UK). HA-coated ULNL had a mean diameter of approximately 100 nm. We refer here HA-ULNL as nanoparticles (NP). Attachment of antibodies to NPs increased the diameters by 20 to 35 nm on average. A zeta potential of −15.9 mV of HA-ULNL is attributable to the carboxylic residues of HA. Attachment of antibodies through primary amines in the antibodies to un-occupied carboxylic residues of HA was likely to neutralize the negative charge. Measurement with ¹¹¹InCl₃-labeled antibodies [82, 83] showed 75-102 antibody molecules per particle. Overall, antibody-coated particles were shown to be homogenous both in sizes, surface charges, and the number of antibodies attached.

Encapsulation of siRNA.

We encapsulated siRNAs to CD4 [84], Ku70 [18], and luciferase (as a control) [85]. siRNAs were synthesized by Dharmacon Inc. and annealed according to the manufacture's instructions. We examined protamine and PEI for condensing siRNAs and the efficacy of siRNA encapsulation. siRNAs at 2000 pmol were condensed by protamine (Abnova GmbH, Heidelberg, Germany) or PEI at room temperature for one hour. Lyophilized liposomes were re-hydrated with Hepes-buffered saline containing condensed siRNAs. We quantified the amount of siRNAs entrapped in the NP with the RiboGreenm Assay (Molecular Probes). Entrapment efficiency was determined as described [47, 76, 77, 79]. In the absence of a condenser, the efficacy of incorporation was around 28.3±1.7%. Condensation with PEI greatly improved incorporation up to 95.9±6.1%. Protamine gave us the efficacy of 77.8±4.4%. Other nanoparticles showed similar results. Because of a concern about the toxicities of a synthetic cationic polymer PEI, we will use protamine, a natural endogenous product used clinically in neutralizing heparin in cardiac surgery.

Silencing in Lymphocytes Constitutively Activated by Agonists.

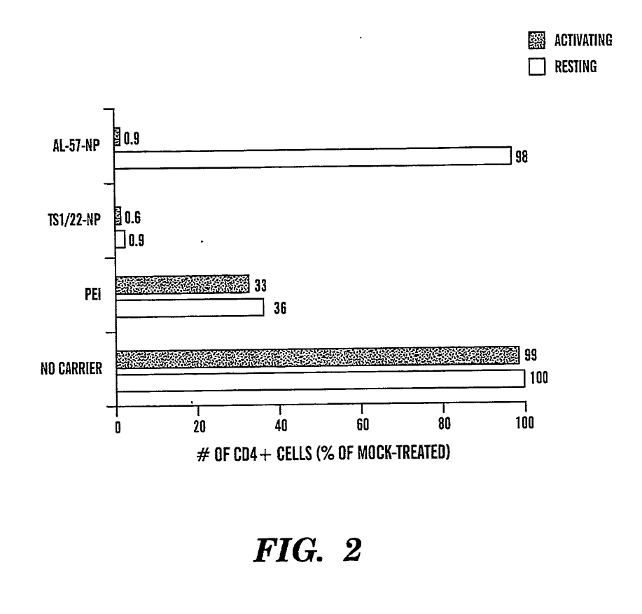

In order to investigate the feasibility of siRNA delivery and gene silencing by AL-57-NP selectively to activated leukocytes, we selected CD4 as a reference molecule. The expression of CD4 on the cell surface is conveniently measured by immunofluorescent cytometry (IFC). Peripheral blood mononuclear cells (PBMC) obtained from healthy volunteers were treated with AL-57-, TS1/22-, or IgG-NPs in RPMI, 10% FCS supplemented with 1 mM MgCl2, 1 mM CaCl2 (resting condition) or 5 mM MgCl₂, 1 mM EGTA plus an activating mab CBRLFA-1/2 (activating condition). AL-57-NP bound to cells only in the activating condition, whereas TS1/22-NP bound cells in both conditions. After 60 hrs, CD4-siRNA incorporated in AL-57-NP almost completely silenced CD4 expression in the activating condition, whereas there was little silencing in the resting condition (FIG. 2). By contrast, CD4-siRNA in TS-1/22-NP showed a robust silencing in both resting and activating conditions. The siRNA without a carrier showed little silencing and transfection by commercial reagent PEI showed a mild reduction of the CD4+ population (FIG. 2). Although we need to obtain a time course and does-response of silencing, the preliminary data showed siRNA delivery and silencing by AL-57-NP selectively to activated lymphocytes.

Silencing in Lymphocytes in which LFA-1 is Constitutively Activated by Physiologic Inside-Out Signaling Through TCR and Chemokine Receptor.

We have shown that the CD4 molecule was silenced by AL-57-NP selectively in cells constitutively activated with Mg2+/EGTA/CBRLFA-1/2 (FIG. 2). These agonists directly affect on the extracellular part of LFA-1 and induce the high-affinity conformation. By contrast, physiological LFA-1 activation is through inside-out signaling, in which T-cell receptor (TCR) engagement or binding of chemokines to their receptors initiates intracellular signaling cascades that eventually impinge on the cytoplasmic tails of LFA-1 and induce the conformational changes of the extracellular part to the high-affinity form [59, 86]. Conversely, binding of ICAM-1 stabilizes the high-affinity conformation of LFA-1 and transduces signals to the cytoplasm (outside-in signaling). Here we demonstrated the ability of AL-57-NP to induce gene silencing selectively in lymphocytes activated by physiologic inside-out signaling. We studied two major pathways that lead to LFA-1 activation, engagement of TCR [87] and chemokine-receptor [88]. To induce sustained activation of LFA-1, we co-immobilized a mAb to CD3 and chemokine CXCL-12 with a LFA-1 ligand ICAM-1 [87, 89-91].

96-well plates were coated with ICAM-1 (10 μg/mL), anti-CD3 mAb (HIT3a, BD Pharmingen) (10 μg/mL), CXCL-12 (5 μg/mL), ICAM-1 plus CXCL-12, or ICAM-1 plus anti-CD3 overnight at 4° C. Plates were blocked with complete media containing 10% FCS. 2×10⁵ cells were added to each well, and treated with CD4-siRNA incorporated in AL-57-, TS1/22, or IgG-NPs. After 60 hrs, cells were harvested and subjected to IFC analysis. For simplicity, we examined CD4-silencing in CD4 T-cells purified by immuno-magnetic beads instead of whole T-cell populations. We used isolated CD4+ T-cells cultured in IL-2. IL-2 treatment alone does not induce the high-affinity LFA-1, but primes lymphocytes to readily respond to stimulation through TCR and chemokine receptor as show below.

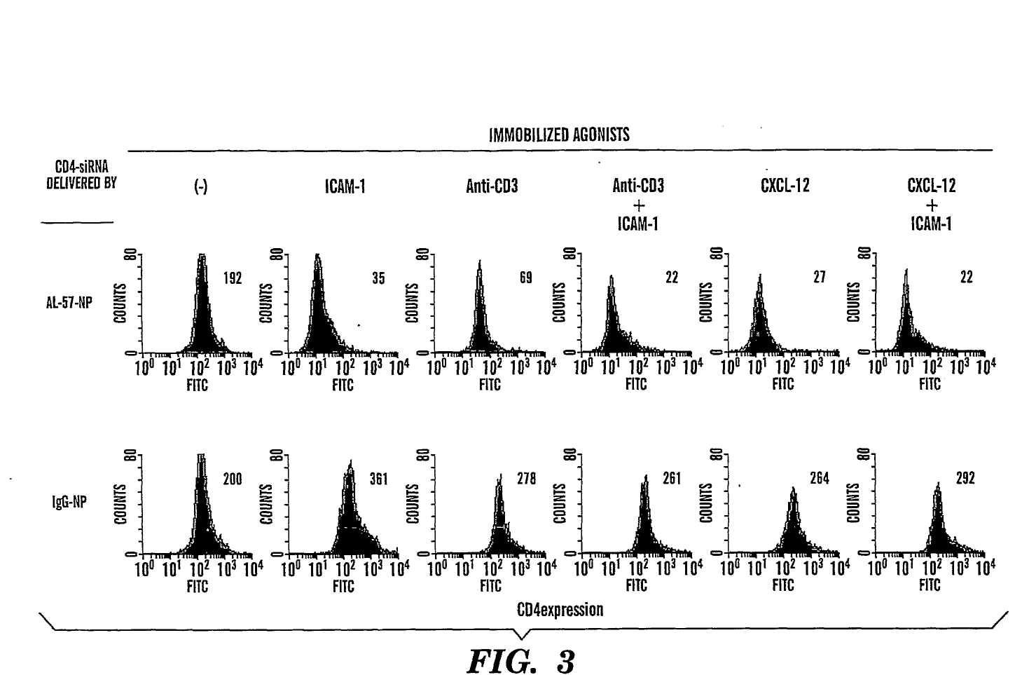

Activation by ICAM-1, CD3, and CXCL12 per se appeared to augment CD4 expression (FIG. 3). The presence of an ICAM-1 substrate induced significant silencing of CD4 by AL-57-NP, but not IgG-NP (FIG. 3). IL-2-treated T-cells exhibit constitutive migration on ICAM-1 substrate ([90] and an observation that we have confirmed the condition used here). Thus, the data suggest efficient siRNA delivery to actively migrating cells on the ICAM-1 substrate, but not IL-2-treated cells settling on a control substrate (FIG. 3). Activation by CD3 cross-linking also showed good reduction of CD4 expression by AL-57-NP. Co-immobilization of anti-CD3 mAb and ICAM-1 showed additive effects. Immobilized CXCL-12 alone induced strong gene silencing by AL-57-NP. Addition of ICAM-1 to CXCL-12 appeared to further enhance silencing. Neither siRNA alone nor siRNA in IgG-NP showed reduction of CD4 expression (FIG. 3).

These data demonstrate that activation of LFA-1 by physiologic inside-out signaling enhanced siRNA delivery and gene-silencing by AL-57-NP. The data here indicate that AL-57-NP preferentially targets active and persistently adhesive cells that express high-affinity LFA-1. IL-2 treatment alone appears insufficient to induce active and persistently adhesive cells. The induction of the active and persistently adhesive cells requires TCR engagement, chemokine signaling, and/or ligand binding. The high selectivity of AL-57-NP to the active and persistently adhesive cells, but not to IL-2-only-treated primed cells, will be advantageous for anti-inflammatory therapies, as the active and persistently adhesive cells are those engaged locally at sites of inflammation.

siRNA Delivery by AL-57-Protamine Fusion Protein (AL-57-PF).

As an alternative and complementary approach to immuno-nanoparticles, we will study siRNA delivery by antibody-protamine fusion proteins. AL-57-PF has several potential advantages. First, the production of AL-57-PF is simple, compared to the production of immuno-nanoparticles that requires multiple steps of surface modifications. Second, as opposed to the multi-valency of AL-57 on immuno-nanoparticles, mono-valent AL-57-PF diminishes the potential to elicit outside-in signaling and unwanted activation upon binding to LFA-1 on the cell surface as investigated in examples 2.1.3 and 2.2. Third, as the fusion protein-siRNA complex is not likely to form particles that might be trapped by the lung and spleen, the tissue-specific siRNA delivery by the fusion protein might exhibit some advantages in vivo compared to the delivery by immuno-nanoparticles.

Production of AL-57-Protamine Fusion Protein and its Binding to Cell Surface LFA-1.

To streamline the production of the fusion protein, we sought to express an AL-57-protamine fusion protein as a single polypeptide in E. coli. In a different project, in which we propose to affinity-mature AL-57 with yeast display, we converted an IgG form of AL-57 that contains the heavy and light chains into a single-chain Fv (scFv) and confirmed intact binding and selectivity of scFv AL-57 to the high-affinity form of LFA-1.

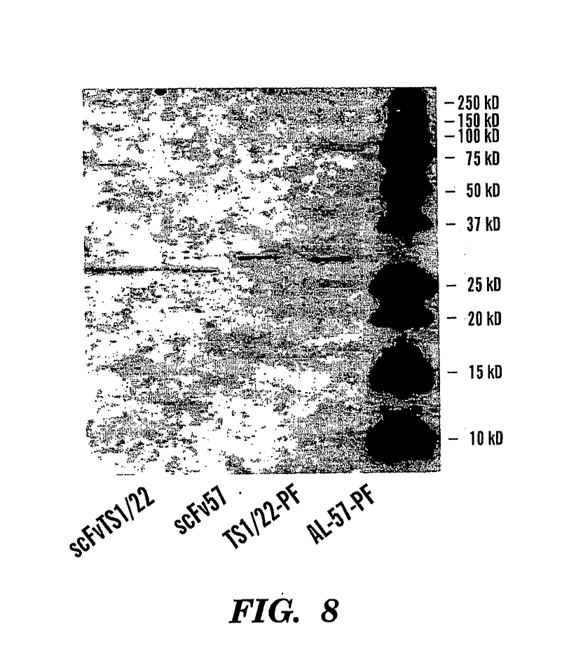

A cDNA fragment containing the scFv AL-57 fused to the N-termini of either full-length or truncated protamine (from residue 8 to 29) was constructed by overlap-PCR and sub-cloned into a vector pET 26b (Novagen) that attaches a 6× histidine tag at the C-termini. The fusion proteins were expressed in E. coli BL21-DE3 (Novagen) and purified from the soluble cytoplasmic fraction with a Ni-NTA affinity column. We have found that the expression of the full-length protamine fusion protein was very low (less than 0.1 mg from one litter of bacterial culture), which is consistent with the poor expression of full-length protamine fusion protein in mammalian cells [92]. In contrast, the expression of the truncated protamine fusion protein was 1 mg/L on average. Therefore, we decided to use the truncated protamine fusion protein in future studies. Fusion proteins were further purified by mono S HR5/5 ion-exchange column (Pharmnacia). FIG. 8 presents an SDS-PAGE showing the proteins with or without protamine on a gel.

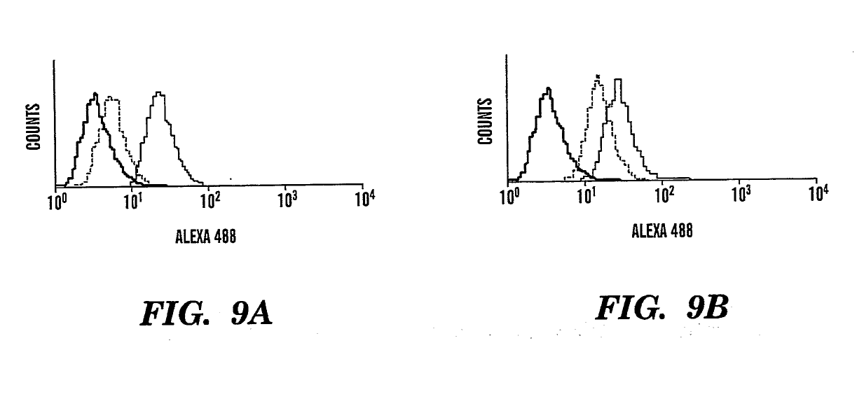

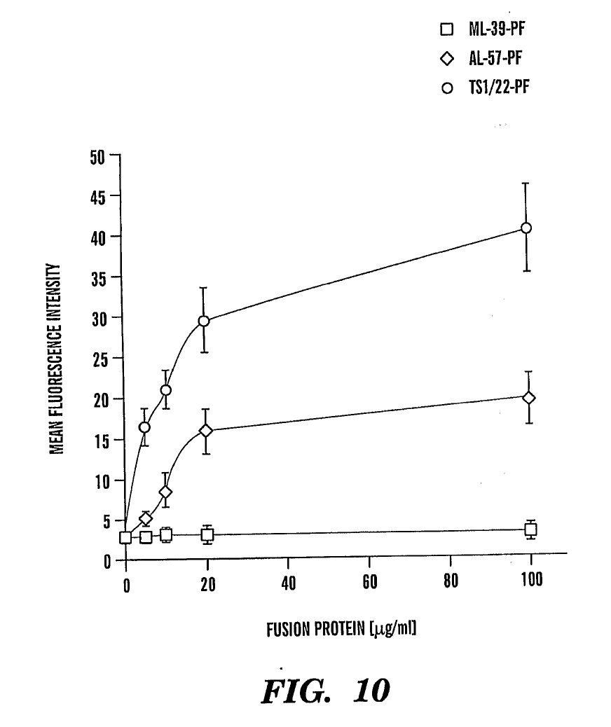

Binding of AL-57- or TS1/22-PF to fresh PBMC. FIGS. 9 and 10 show binding to freshly isolated peripheral bllod mononuclear cells in naïve and in activated conditions. Looking at the figures it becomes clear that AL-57-PF binds to LFA-1 on the cell surface of the leukocytes only upon activation, whereas TS1/22-PF binds in either active or näive conditions. ML39-PF, which served as an isotype control, did not bind at all.

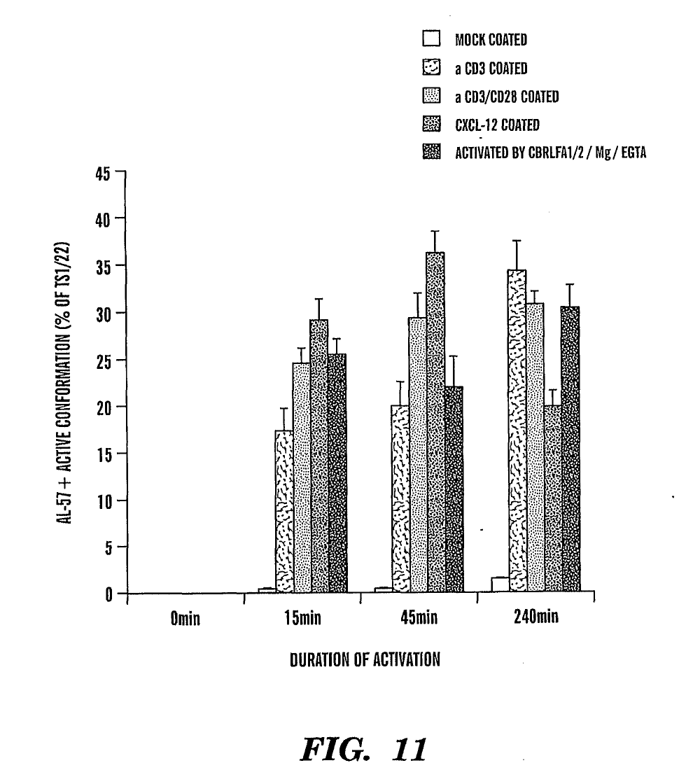

Sustained activation in the presence of immobilized agonists for up to 4 hours is presented in FIG. 11. AL-57⁺ active conformation (as % of TS1/22) is presented. The data clearly show that AL-57-PF is activated also by immobilized agonists such as anti-CD3, anti-CD3/CD28, and CXCL-12 as well as by activating antibody such as CBRLFA1/2+ Mg+ EGTA. One can also see the effect of activation in each time point by mock coating the plastic plates, i.e., no conformational changes occur without any activation.

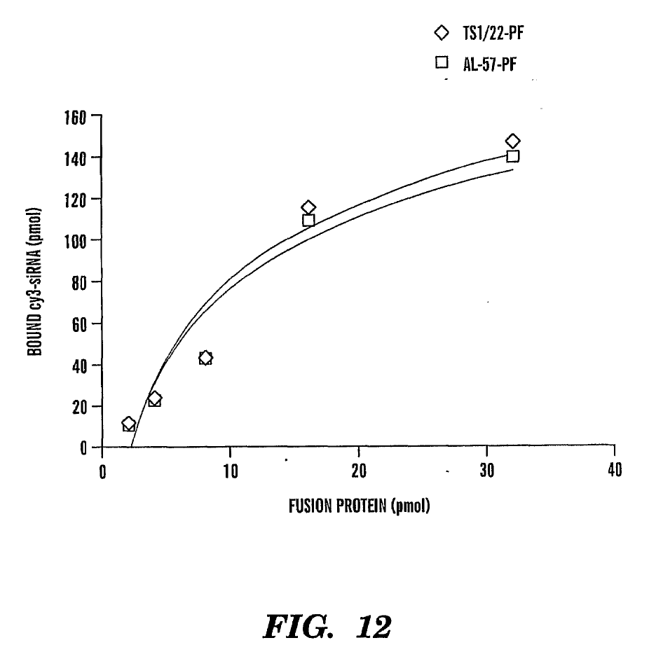

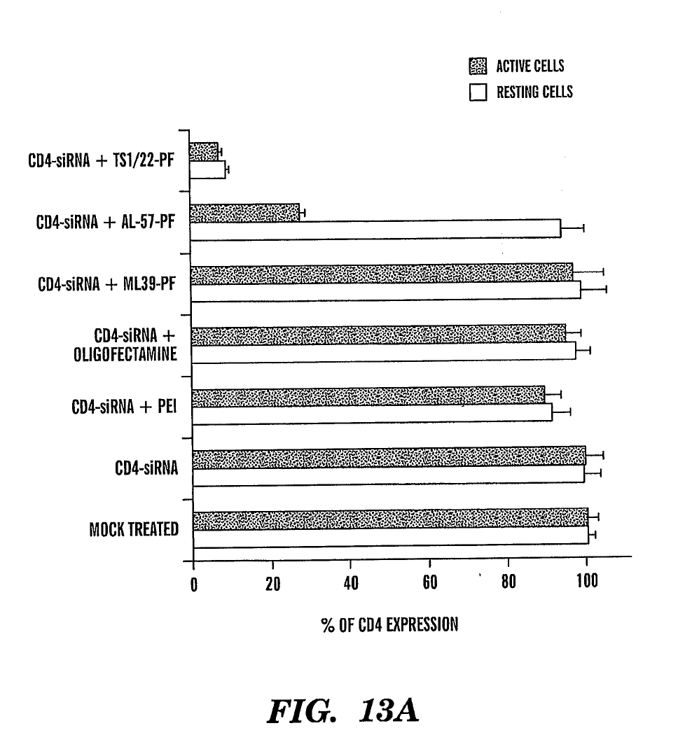

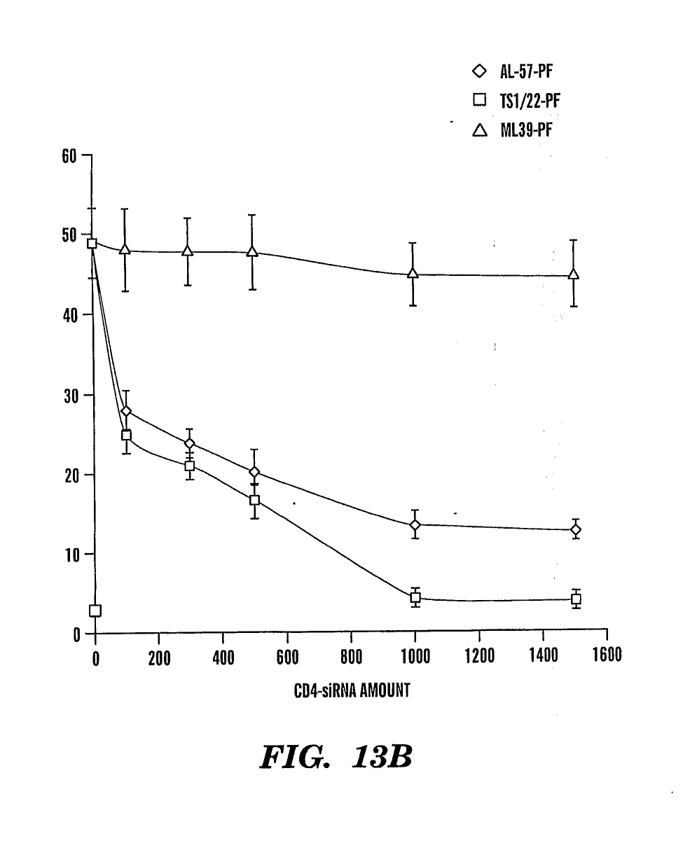

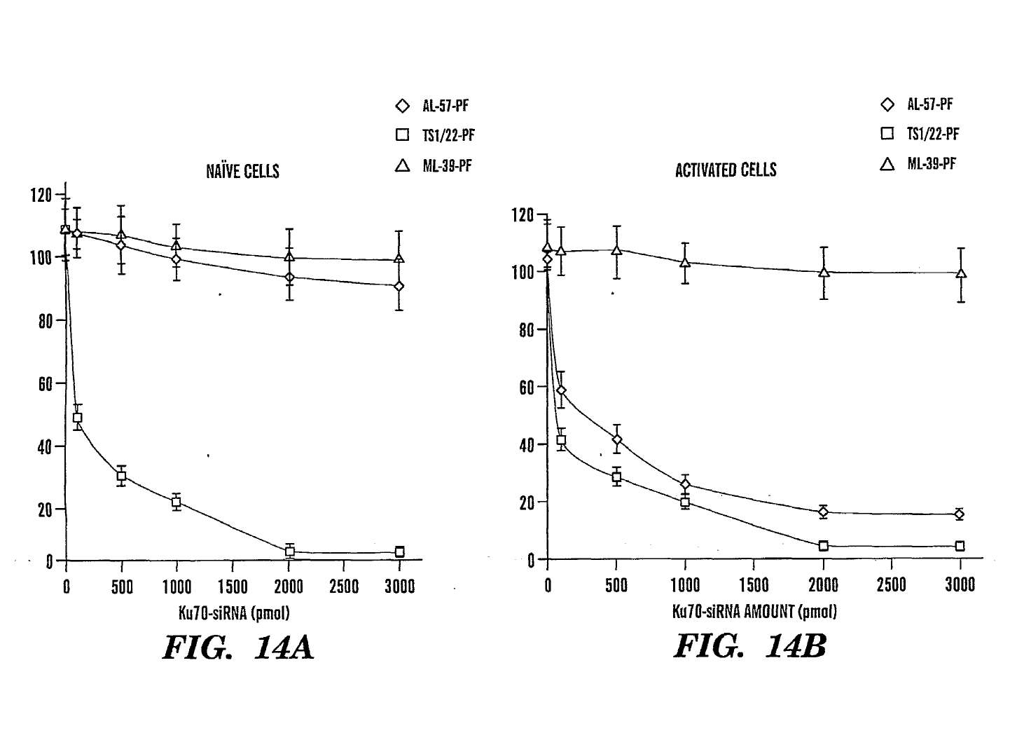

Stoichiometric analysis shows (FIG. 12) that approximately 5 molecules of siRNA are bound to 1 molecule of AL-57-PF or TS1/22-PF. Table I gives additional indication for condensation through protamine. siRNAs in solution (PBS, pH 7.4) retain a negatively charge surface. The charge is flipped from mildly negatively to mildly positively upon condensation with either AL-57-, TS1/22- or ML39-PF. The size measurements also indicate the condensation of the siRNAs with the fusion proteins.

TABLE I
Size and zeta potential measurements of siRNA and fusion proteins

| Carrier | Size (nm) | Zeta potential (mV) |
|---|---|---|
| Naked siRNA | 678 ± 102 | −43.9 ± 5.1 |
| AL57-PF + siRNA (1:5) | 120 ± 22 | 28.1 ± 4.2 |
| TS1/22-PF + siRNA (1:5) | 104 ± 25 | 33.1 ± 3.7 |
| ML39-PF + siRNA (1:5) | 115 ± 32 | 25.1 ± 5.2 |

Complexes were formed at room temp. over 30 min in PBS, pH 7.4
Size and zeta potential were measured using Malvern zetasizer 3000 (Malvern, MA) at pH 7.4 in PBS.
Luciferase-siRNA (2 μg) was used as a representative siRNA.
Data are presented as average ± standard deviation from six independent experiments.

Silencing by AL-57-PF.

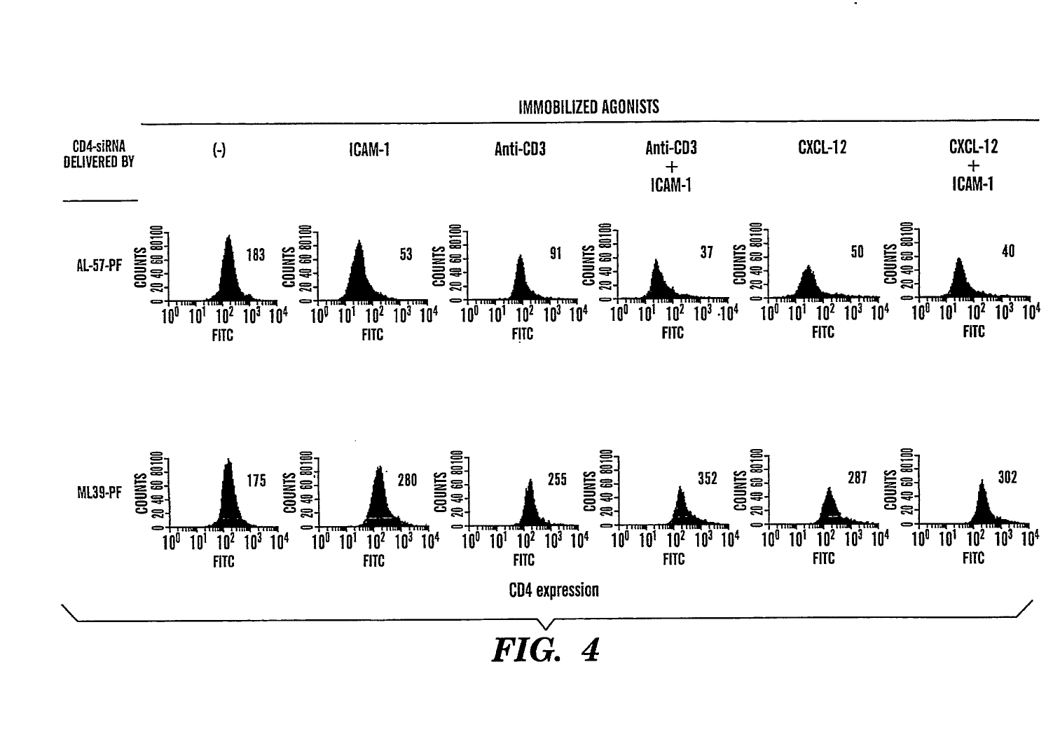

We investigated gene silencing by AL-57-PF selectively in activated leukocytes in vitro. Stoichiometry analyses done as described [1,8] showed that up to five Cy3-siRNA molecules bound to each AL-57-PF molecule, consistent with previous results [18]. We examined delivery of CD4-siRNA in PBMC as in the section “Silencing in lymphocytes constitutively activated by agonists”. CD4-siRNA was complexed with AL-57-PF or control non-binding scFv ML39-protamine fusion protein (ML39-PF) [18] and added to cells either in resting or activating condition. After culturing for 60 hr, we analyzed the CD4 expression. CD4-siRNA delivered by AL-57-PF showed a dose-dependent silencing of the CD4 molecule in the activating condition (FIGS. 4 & 13). At 1000 pmol, CD4 was almost completely silenced by AL-57-PF, showing much stronger effects than transfection with PEI or Oligofectamine™. Importantly, the AL-57-PF-directed delivery showed little silencing in the resting condition. siRNA alone or delivery by ML-39-PF induced no silencing (FIGS. 4 & 13). siRNA delivered by TS1/22-PF gave the most effective silencing.

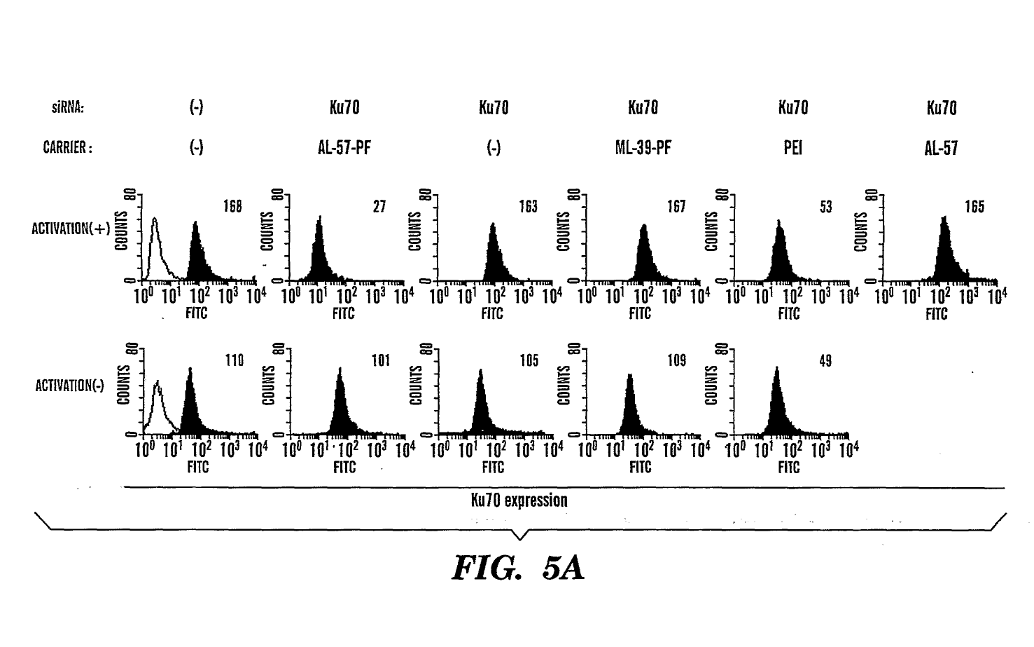

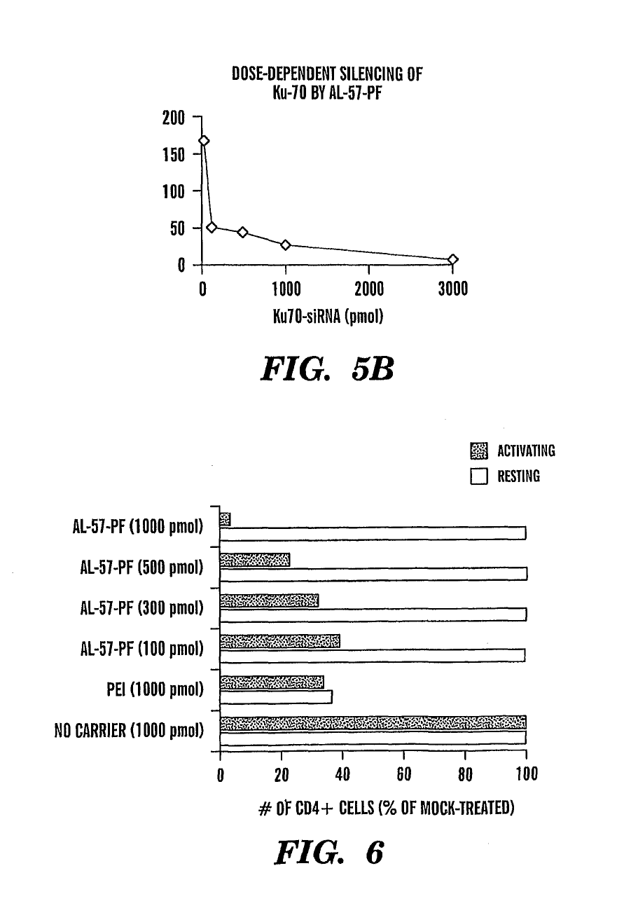

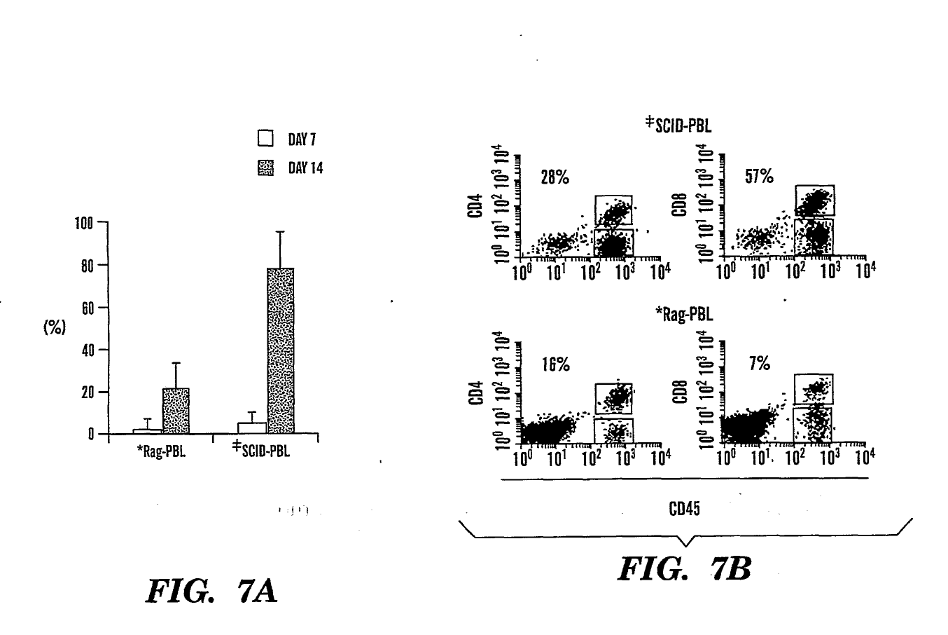

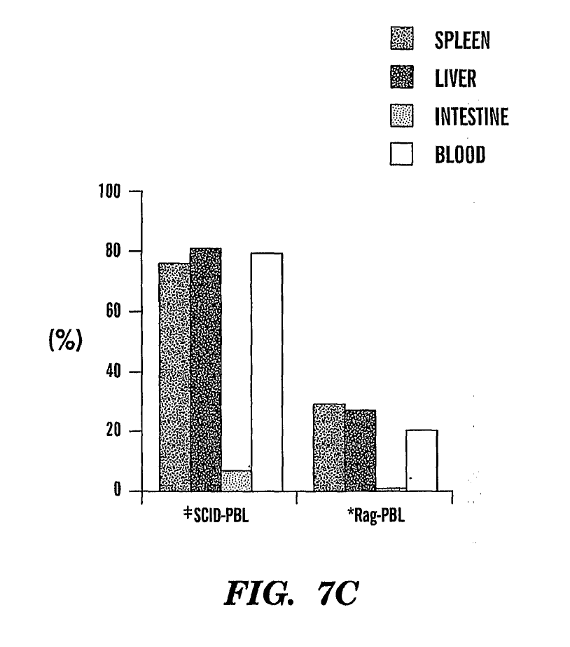

In order to generalize our results on CD4 silencing, we investigated another reference molecule Ku70, a ubiquitously expressed nuclear protein, which allows us to examine the effects of silencing in all types of cells. Lymphocytes were treated with Ku70-siRNA complexed with AL-57-PF, TS1/22-PF or ML39-PF either in the resting or activating condition as in the section above entitled “Silencing in lymphocytes constitutively activated by agonists”. After culturing for 60 hr, cells were fixed and permeablized as described [18], and the expression of Ku70 was examine by IFC using mAb to Ku70. Substantial silencing was induced by Ku70-siRNA delivered by AL-57-PF in the activating condition (FIGS. 5 & 14). By contrast, virtually no silencing by AL-57-PF was observed in the resting condition. Neither Ku70-siRNA delivery by ML39-PF nor scFv AL-57 antibody without a protamine moiety induced silencing (FIG. 5). These results (FIGS. 4 and 5) demonstrated the ability of AL-57-PF to selectively induce gene silencing in the activated leukocytes that express the high-affinity LFA-1.

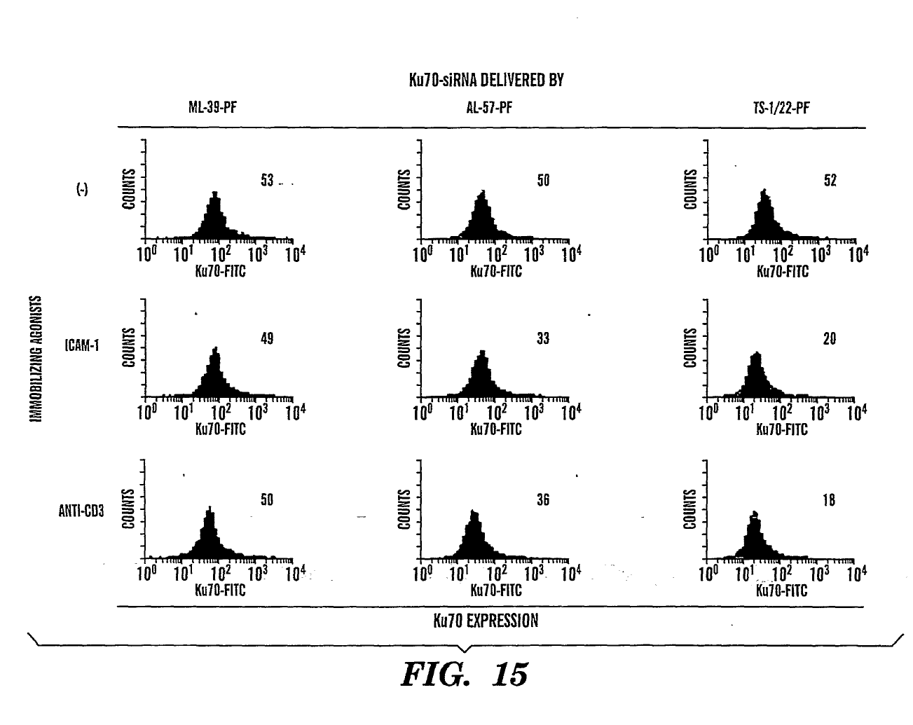

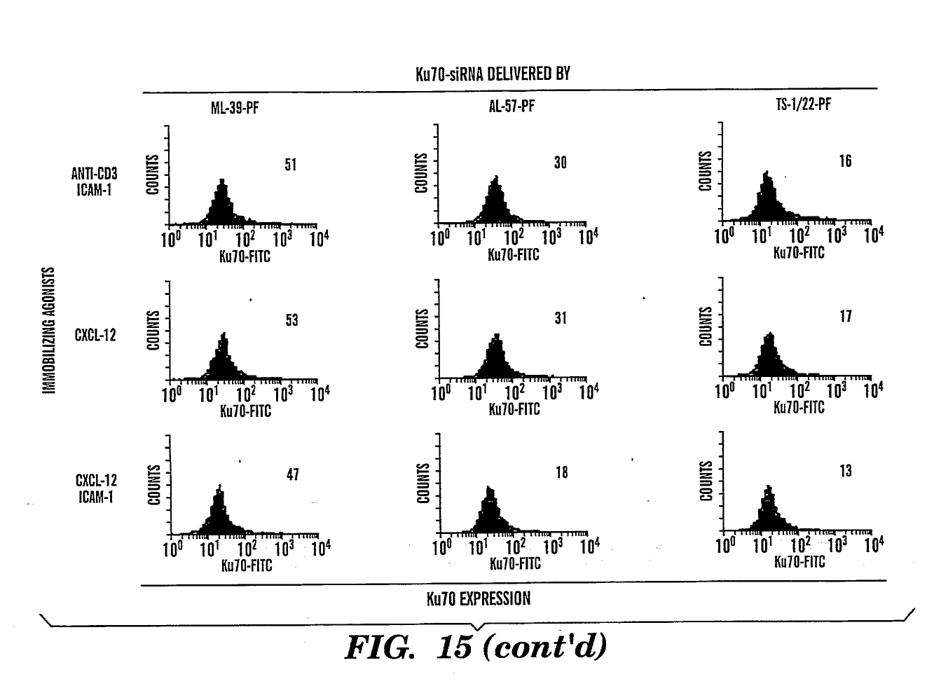

We next turned to studies of LFA-1 activated by physiologic inside-out signaling as in the section above entitled “Silencing in lymphocytes in which LFA-1 is constitutively activated by physiologic inside-out signaling through TCR and chemokine receptor”. IL-15 cultured lymphocytes activated by anti-CD3 mAb, anti-CD3/CD28 or CXCL12 and treated with Ku70-siRNA complexed with AL-57-, TS1/22- or ML39-PF. AL-57-PF induced silencing only when T-cells were activated through the immobilized agonists (FIG. 15). Mild IL-15 treatment used here did not induce AL-57-PF-directed gene silencing (FIG. 15). Neither siRNA alone nor siRNA delivered by ML39-PF showed reduction of Ku70 expression (FIG. 15). These results indicate that like AL-57-NP, AL-57-PF targets the activated and persistently adherent leukocytes that express the high-affinity form of LFA-1.

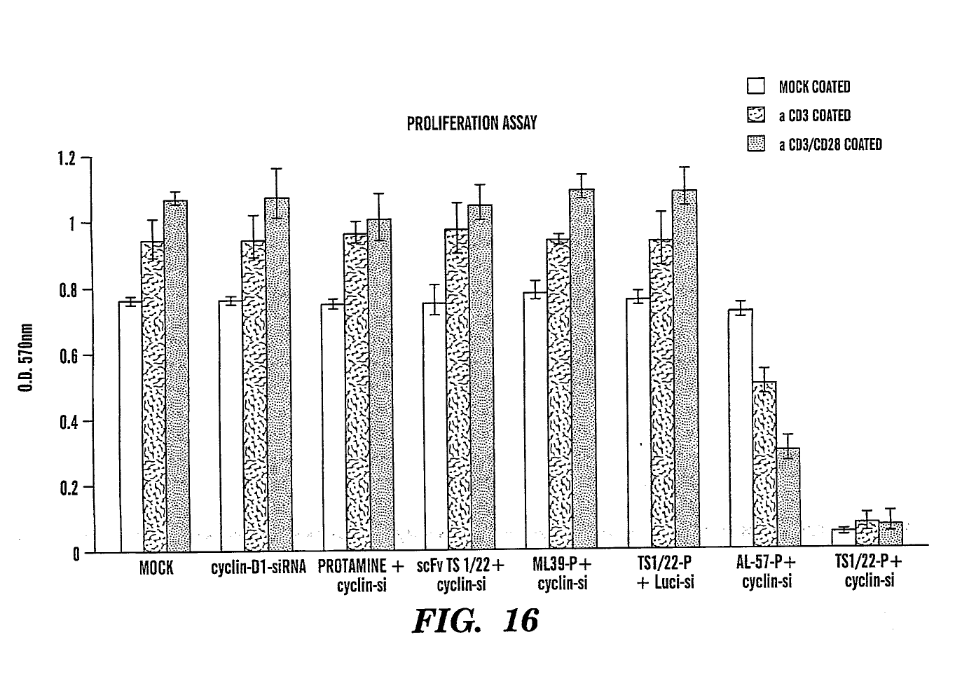

Inhibited proliferation of IL-15 cultured lymphocytes on immobilized agonists by cyclin-D1-siRNA delivered by TS1/22-PF and AL-57-PF is presented in Supplementary FIG. 9. As clearly showed delivery of cyclin-D1-siRNA via TS1/22-PF stopped the proliferation of IL-15 cultured lymphocytes on mock and on immobilized agonists. AL-57-PF was highly selective in targeting cells that were activated by immobilized agonists. TS1/22 (scFv) nor ML39-PF did not cause any inhibition of proliferation to the cells immobilized with different agonists. (FIG. 16).

AL-57-PF and TS1/22-PF were each individually labeled with Alexa 488 dye. siRNA against CCR5 was labeled with cy3 dye. Cells were immobilized on CXCL-12 or anti-CD3 and were treated with AL-57-PF or TS1/22-PF that were previously condensed Cy3-siRNA (against CCR5). Cy3-labeled siRNA and Alexa 488-labeled TS1/22-PF or AL-57-PF. Confocal microscopy was used to investigate the ability of labeled fusion proteins to bind and deliver Cy3-siRNA selectively to activated lymphocytes. IL-15 cultured lymphocytes were examined and photographed at 45 minutes and 240 minutes following exposure of activated lymphocytes to the fluorescently labeled fusion protein-siRNA complexes. Alexa-488 (AL). Four hours after exposure of activated lymphocytes to the fluorescently labeled fusion protein-siRNA complexes, Alexa-488-AL-57-PF was distributed to both the plasma membrane and internal punctuate structures, whereas Cy3-siRNA was predominantly intracellular, colocalizing with the fusion protein. The conformation-sensitive fusion protein AL-57-PF did not transducer unactivated lymphocytes. As expected, T cells treated with Alexa488-TS1/22-PF internalized Cy3-siRNA with a similar staining pattern, but uptake was independent of cell activation. AL-57-PF selectively targeted the cells that were activated by CXCL-12 or anti-CD3 and delivered fluorescently—siRNA. When näive cells were used no siRNA delivery and no binding was observed. When TS1/22-PF was used näive as well as activated cells were being used.

### Example 2.1

### Gene Silencing by AL-57-NP In Vitro

### Example 2.1.1

### Silencing in Constitutively Activated Lymphocytes in Heterogeneous Populations

Rationale.

Subpopulations of T-lymphocytes with oligo-clonality have been shown to be activated and proliferate in autoimmune and inflammatory disorders [98-103]. Therefore, in addition to studying the resting and activating conditions separately as above in “Silencing in lymphocytes constitutively activated by agonists” and “Silencing in lymphocytes in which LFA-1 is constitutively activated by physiologic inside-out signaling through TCR and chemokine receptor”, we will investigate the selective delivery targeting the high-affinity LFA-1 in heterogeneous populations in which cells that express the high-, low-, and probably intermediate-affinity LFA-1 co-exist. We hypothesize that in heterogeneous leukocyte populations, AL-57-NP will be able to deliver siRNA selectively to the activated leukocytes that express the high-affinity LFA-1. To progress from simple to more complex, physiologically relevant setting, we will perform three sets of experiments where only part of the leukocyte populations expresses the high-affinity LFA-1:1) co-culturing CBRLFA-1/2-treated cells with untreated cells, 2) activating subpopulation of T-cells that express TCR Vβ3 by cross-linking with mAb to TCR Vβ3, and 3) whole blood samples where T-cells and monocytes/neutrophils are preferentially activated by anti-CD3 mAb and TNF-α, respectively. The experiments outlined below will show the selectivity of siRNA delivery by AL-57-NP in a manner relevant to inflammation in vivo.

We will use another activation-dependent mAb KIM127 as a complementary approach to determine the high-affinity form of LFA-1. KIM127 maps to the leg domain of the β₂ subunit of LFA-1 (α_{L}β₂) and reports the early phase of conformational changes that precedes the high-affinity I domain that AL-57 reports [62, 104, 105]. Thus, KIM127 defines high-affinity form of LFA-1 broader than AL-57 (the exposure of KIM127 epitope is required but not sufficient to express AL-57 epitope). More importantly, AL-57 and KIM127 bind distinct epitopes and can bind to the active form of LFA-1 simultaneously without competing each other. Simultaneous fluorescent staining of cells with KIM127-FITC (fluorescein isothiocyanate) and AL-57-NP-Cy3 allows us to confirm that binding of AL-57-NP is selective to high-affinity LFA-1-expressing cells (KIM127high).

Methods.

For simplicity, we will use primary näive CD4+ T-cells in (i) and (ii). CD4+ T-cells will be isolated from PBMC with magnetic beads as described. In (iii), whole blood obtained form healthy volunteers will be used.

(i) Activation by CBRLFA-1/2

One group of cells will be fluorescently labeled green with CFSE (carboxyfluorescein succinimidyl ester, Invitrogen) as described [106] and activated with 10 μg/ml CBRLFA-1/2. After washing three times to remove unbound antibody in solution, cells will be resuspended in a complete media that contains Mg2+/Ca2+. CBRLFA-1/2-bound, CFSE-labeled cells (active) will be co-cultured in Mg2+/Ca2+ with the same number of CBRLFA-1/2-untreated and unlabeled cells (inactive). AL-57-, TS1/22, or IgG-NPs containing CD4-siRNAs will be given to the co-cultures. After indicated time up to 60 hr, cells will be harvested and subjected to IFC to study expression of CD4 using PE (phycoerythrin)-labeled mAb. Changes of CD4 expression in CFSE+ (activated) and CFSE− (resting) populations will be analyzed. In some experiments, CBRLFA-1/2 untreated näive cells will be labeled with CFSE.

(ii) Activation by Cross-Linking of TCR Vβ3

The Vβ3+ population constitutes 5 to 10% of total peripheral blood T-cells [107]. Cross-linking of TCR Vβ3 activates and transduces the inside-out signaling to LFA-1 only in TCR Vβ3+T-cells. A mitogenic mAb to TCR Vβ3 (JOVI-3, Ancell) or isotype control IgG will be immobilized in 96-well plates as described. We will titrate the concentration of the mAb so that the high-affinity LFA-1 will be induced. 2×10⁵ cells will be added to each well, and treated with CD4-siRNA incorporated in AL-57-, TS1/22- or IgG-NPs. At indicated time points up to 60 hr, cells will be harvested and subjected to IFC to examine expression of CD4 in TCR Vβ3-positive or negative populations using anti-CD4-FITC and anti-TCR Vβ3-PE antibodies. In some experiments, we will determine if cross-linking with immobilized anti-Vβ3 will induce the high-affinity form of LFA-1 by IFC with KIM127-FITC and anti-TCR Vβ3-PE. We will stain anti-TCR Vβ3-treated cells with KIM127-FITC and AL-57-NP-Cy3 to confirm that binding of AL-57-NP is selective to KIM127^{high} cells that express the high-affinity LFA-1. AL-57-NP-Cy3 will be prepared by attaching Cy3-labeled AL-57 to nanoparticles as described.

(iii) Delivery to Lymphocytes, Monocytes, and Neutrophils in Whole Blood Samples.

As the affinity states and the kinetics of LFA-1 activation in lymphocytes, monocytes, and neutrophils may differ, we will investigate the siRNA delivery by AL-57-NP in heterogeneous leukocyte populations in whole blood. We will selectively activate either lymphocytes by immobilized anti-CD3 mAb or monocytes and neutrophils by TNF-α. TNF-α treatment increases the affinity of leukocyte integrins in neutrophils [108] but not in lymphocytes. For reference, LFA-1 in all leukocytes will be activated by Mn²⁺ or PMA. We will first validate the experiments by examining the differential increase of LFA-1 affinity in each population by mAbs AL-57 and KIM127 in various activating conditions listed here. Then, we will treat whole blood samples with Ku70-siRNA in AL-57-, TS1/22-, and IgG1-NPs in the presence of different agonists. Samples will be subjected to IFC analysis at 24, 48, and 60 hrs after addition of siRNA. Considering the short lifetime of neutrophils, in some experiments to avoid culturing cells for many hours, we will incubate samples with Cy3-labeled immuno-nanopartcles for 30 min at 37° C. and examine the differential binding of Cy3-AL-57-NP. Expression of Ku70 and binding of Cy3-immuno-nanoparticles to each subset will be studied by IFC as mentioned above.

Anticipated Results & Potential Pitfalls/Alternative Approaches.

Regarding activation by CBRLFA-1/2, we expect that AL-57-NP will knock down CD4 expression only in CBRLFA-1/2-treated cells, whereas TS1/22-NP will attenuate expression in both CBRLFA-1/2-treated and untreated cells. CBRLFA-1/2 in Mg/Ca, which is less stimulatory than CBRLFA-1/2 in Mg/EGTA, was confirmed to induce binding of AL-57-NP to T-cells (not shown). Thus, CBRLFA-1/2 treated cells will be sufficiently active to support binding of AL-57-NP. CBRLFA-1/2-bound LFA-1 will be recycled and internalized and there is no free CBRLFA-1/2 in media during co-culturing; we are aware that the activation of LFA-1 might be less strong than media containing free mAb in solution. Cells activated by CBRLFA-1/2 might secrete cytokines such as IL-2 and upregulate cell surface expression of ICAM-1, stimulating in co-culture CBRLFA-1/2-untreated näive cells through cytokines and cell-cell contacts. Although we are aware of this secondary activation of CBRLFA-1/2-untreated näive cells, we anticipate that the secondary activation will be mild and not sufficient to induce the persistently high-affinity LFA-1, as IL-2-treatment alone was not sufficient to induce the active and persistently adhesive lymphocyte as shown in the preliminary data (FIGS. 2 & 3). Should the secondary activation be strong enough to induce the high-affinity LFA-1, the high-affinity LFA-1-expressing cells, whether activated primary or secondary, will be identified by KIM127. Therefore, IFC analyses with KIM127-PE and anti-CD4-PerCP (Peridinin chlorophyll protein) will enable us to study silencing selective to the high-affinity LFA-1. KIM127 and CBRLFA-1/2 do not compete each other [62]. We are also aware that similar secondary activation may occur to TCR Vβ3-cells in (ii), lymphocytes in TNFα-treatment in (iii), and monocytes/neutrophils in anti-CD3 mAb treatment in (iii). We will manage the secondary activation in (ii) and (iii) as for CBRLFA-1/2-treatment in (i). In addition, we will consider the possibility that bi-valency of antibody might allow cell-bound CBRLFA-1/2 to bind to neighboring näive cells and induce activation. We will use Fab form of CBRLFA-1/2 in some experiments to rule out this possibility.

Regarding activation by cross-linking TCR Vβ3, we anticipate that CD4-siRNA in AL-57-NP will achieve silencing only in TCR Vβ3+ cells. Our preliminary data showed that 10% of peripheral T-cells were TCR Vβ3-positive, and treatment with immobilized anti-Vβ3 mAb for 3 days expanded the Vβ3+ population to 15%. Should we be unable to induce sufficiently robust LFA-1 activation by TCR Vβ3 cross-linking alone, we will co-immobilize sub-mitogenic concentrations of anti-CD3 mAb. We will titrate the concentrations of anti-CD3 mAb and monitor LFA-1 activation with mAbs AL-57 and KIM127. We will determine the concentrations at which LFA-1 activation is maximized in TCR Vβ3+ cells while LFA-1 is latent in TCR Vβ3− cells.

The experiments using whole blood will allow us to study not only lymphocytes but also neutrophils and monocytes. Activated neutrophils and monocytes express high-affinity LFA-1, to which AL-57 will deliver siRNAs. As these cells play important roles in inflammatory tissue damage [70, 109], the ability to target them upon activation will be advantageous. Neutrophils and monocytes may show an increase background uptake of nanoparticles. As mentioned below (examples 2.1.2 and 2.3.3), the replacement of intact IgG with Fab will eliminate Fc-receptor-mediated binding. Fab fragments will be prepared by papain digestion. Covalent attachment will be performed as described above. In addition, should we observe substantial hyaluronan-associated background binding, we will considering other surface modifications such as PEG for the purpose of generating long-circulating particles.

### Example 2.1.2

### IFN-Response

Rationale.

Delivery of siRNA can potentially elicit interferon responses either through the cytosolic dsRNA-activated protein kinase PKR or binding to Toll-like receptors 3 and 7 that recognize RNA on the cell surface or in endosomes [110, 111]. Although naked siRNA induced no detectable interferon response upon injection to mice, administration with cationic-lipid based carriers led to activation of STAT1 [112, 113]. A recent report showed that the majority of the non-specific silencing elicited by siRNA formulated in cationic lipid (Lipofectamine 2000) came from the cationic lipid component [114]. This non-specific inflammatory response could result in a general inhibition of protein translation and proinflammatory gene expression, interfering with interpretations of results as well as potentially harming patients. Although our nanoparticles do not contain cationic lipids, we seek to rule out the induction by the nanoparticles of the non-specific inflammatory responses. We will therefore examine the interferon responses by looking at mRNA expression of interferon-β, and two key interferon responsive genes, 2′,5′-oligoadenylate synthetase (OAS1) and Stat-1 [18].

Quantitative RT-PCR.

We will examine T-cells treated with siRNA alone or siRNA incorporated in immuno-nanoparticles (AL-57-, TS1/22-, and human and mouse IgG-NP) either in the activating or resting conditions. We will harvest cells at 24 and 48 hrs after delivery of siRNAs (CD4- and Ku70-siRNAs), isolate total RNA with Trizol™ and, using an iCycler instrument (Biorad) and a SYBR green (Molecular Probes), perform quantitative RT-PCR for IFN-γ, OAS1, STAT1, and GAPDH as described [18]. Human macrophage-like cell-line THP-1 will be included to study IFN response in macrophages by immuno-nanoparticles. A positive IFN response will be induced by treating THP-1 cells with polyriboinosinic polyribocytidylic acid [18].

Anticipated Results & Potential Pitfalls/Alternative Approaches.

We do not anticipate the induction of interferon responses. Should we see induction, we will titrate down the amount of siRNAs. Alternatively, we will consider to use the chemical modification of siRNAs that was shown to eliminate the induction of interferon in siRNA delivery by untargeted liposomes [41]. It was shown that the primary IFN-responding cell types are plasmacytoid dendritic cells (pDCs), which express TLRs3 and 7 [110]. Should pDCs express the high-affinity LFA-1, chemically modified siRNAs is desirable for in vivo application.

### Example 2.1.3

### Impact on Integrin Function and Signaling

Rationale.

As mAbs AL-57 and TS1/22 are function-blocking antibodies, AL-57- and TS1/22-NP will inhibit LFA-1-mediated cell adhesion to ICAM-1. This antagonistic activity might result in additive or synergetic anti-inflammatory effects along with siRNA gene-silencing of inflammatory mediators. It is anticipated that blocking mAb and siRNAs will provide therapeutic synergy [115]. In addition to inhibition of LFA-1 function by antibodies, cross-linking of LFA-1 by antibodies can induce outside-in signaling as ligands do [87, 116]. We therefore consider the possibility that AL-57- and TS1/22-NPs might induce signaling through LFA-1. Multi-valency of antibodies on nanoparticles might enhance the signaling by inducing LFA-1 clustering. As immuno-nanoparticle-induced LFA-1 signaling will modify lymphocyte function, we will investigate inhibitory as well as stimulatory activity of AL-57- and TS1/22-nanoparticles on T-cells.

Adhesion Assay

We will confirm that AL-57- and TS1/22-NPs inhibit LFA-1-ICAM-1 interaction. Cell adhesion assay to an ICAM-1 substrate using 96-well plates will be done as described [108, 117]. T-cells will be activated by either Mg2+/Ca2+ plus CBRLFA-1/2 or Mg2+/EGTA plus CBRLFA-1/2. We will treat T-cells with AL-57-, TS1/22-, and IgG-NPs at different concentrations. We will include samples treated with free mAbs AL-57, TS1/22, and control IgGs for comparison.

Costimulatory Activity

In order to study potential stimulatory or inhibitory capacity of immuno-nanoparticles binding to LFA-1, we will analyze proliferative responses of T-cells, which reflect global T-cell activation [118]. We will examine whether AL-57- and TS1/22-NPs will modify proliferation and IL-2 production of T-cells in response to CD3-cross-linking with or without ICAM-1. Anti-CD3 mAb at mitogenic (10 μg/ml), sub-mitogenic (0.1 μg/ml) or null (0 μml) concentrations will be immobilized in 96-well plates with or without ICAM-1 (10 μg/ml). T-cells will be added to wells that have immobilized anti-CD3 mAb and/or ICAM-1. Different concentrations of AL-57-, TS1/22-, or IgG-NPs that either contain or do not contain control luciferase-siRNA will be added to T-cells. T-cells will be cultured for three days. Proliferation will be examined by [3H]-incorporation [118]. IL-2 secreted into media will be measured by ELISA. For comparison in some experiments, soluble mAbs AL-57 and TS1/22 will be used instead of immuno-particles.

Anticipated Results & Potential Pitfalls/Alternative Approaches.

We expect that AL-57- and TS1/22-NPs will inhibit cell adhesion to ICAM-1, as free mAbs AL-57 and TS1/22 block the LFA-1-ICAM-1 interaction (FIG. 1 and [119]). Therefore, we anticipate that AL-57- and TS1/22-NPs will suppress T-cell proliferation in response to anti-CD3 mAb plus ICAM-1 by blocking co-stimulation through LFA-1-ICAM-1. AL-57- and/or TS1/22-NPs might exhibit stimulating effects. However, we expect that as binding to an ICAM-1 substrate induces macro-clustering [117] eliciting strong signaling, inhibition of adhesion to an ICAM-1 substrate will dominate over induction of signaling by the immuno-nanoparticles. In addition, we foresee that the active LFA-1-selective AL-57-NP will induce, if any, less co-stimulatory activity than TS1/22-NP, as the number of LFA-1 cross-linked by mAbs on the cell surface will be less in AL-57-NP, as the active LFA-1 represents only a subpopulation of the total LFA-1 [91, 120]. Should we observe stimulatory effects by AL-57-NP, we will rule out immune responses mediated by Fc portion of IgG by testing Fab AL-57- or TS1/22-coated immuno-nanoparticles as described. Alternatively, we will consider an alternative delivery means, AL-57-protamine fusion protein, in which binding is monomeric and is expected to provide less potent stimulation for the induction of the outside-in signaling as proposed Aim 2 (AL-57-protamine fusion protein).

### Example 2.2

### siRNA Delivery by AL-57-Nanoparticles In Vivo

### Example 2.2.1

### Leukocyte Activation in NOD/Lt-scid IL2rγ^{null}-hu-PBL

Rationale.

NOD/Lt-scid-hu-PBL and NOD/Lt-scid B2mnull-hu-PBL mice showed a transient xenogenic activation of engrafted T-cells for a duration of 2 to 3 weeks, followed by an anergic state [93]. Biphasic activation allows us to study activated as well as anergic T-cells in the same model depending on the timing of the analysis of the engrafted cells. However, the activation kinetics of human LFA-1 in engrafted T-cells in humanized mice is unexplored. We hypothesize that 1) NOD/Lt-scid IL2rγ^{null}-hu-PBL will show a similar biphasic activation of engrafted T-cells; 2) at least some of activated T-cells engrafted in the mice will express the high-affinity LFA-1; 3) levels of LFA-1 activation will decrease as anergy is induced. We will determine in our experimental setting the existence of both a stimulatory phase in which human LFA-1 is in the high-affinity form, and an anergic phase in which human LFA-1 is in latent form.

Methods.

In the following sections, we will study LFA-1 activation using activation-dependent mAbs AL-57 and KIM127 for identifying high-affinity form of LFA1, as well as activation-insensitive non-blocking mAb TS2/4 for total LFA-1. These three antibodies bind to distinct LFA-1 domains without competing each other, allowing simultaneous binding.

Analyses of the Activation Status of LFA-1.

We will study peripheral lymphocytes as well as cells isolated from tissues such as spleen, liver, lung and gut as in Example 1, in the section entitled “Development of humanized mice for studying AL-57-guided delivery in vivo”. As the expression of CD25 (IL-2-receptor) for primed lymphocytes was reported to peak between 7th and 14th day [93], we will study samples at day 1, 3, 7, 14, 21, 28 to determine the stimulatory and anergic phases. We will examine expression of AL-57 and KIM127 epitopes in CD45+ total human leukocyte and TS2/4+ total LFA-1-positive populations. In some experiments, correlation of expression of AL-57 and KIM127 epitopes with that of CD25 will be studied, as active and persistently adhesive cells (AL-57^{high} and/or KIM127^{high}) represent part of primed populations (CD25+).

Binding of AL-57-NP and AL-57-PF Ex Vivo.

After determining the kinetics of LFA-1 activation, we will examine the binding of AL-57-NP and AL-57-PF to engrafted lymphocytes isolated from PBMC and tissues ex vivo. Before administering to mice, we seek to confirm that these delivery vehicles will bind to the lymphocytes only in the stimulatory phase. Binding of AL-57-NP and AL-57-PF to hu CD45+ as well as TS2/4+ cells will be investigated by IFC using fluorescently labeled antibodies to human IgG and protamine, respectively. For comparison, TS1/22-NP and IgG-NP, as well as TS1/22-PF and control non-binding ML39-PF will be included.

Anticipated Results & Potential Pitfalls/Alternative Approaches.

As expression of CD25 in engrafted human T-cells peaked between 7th and 14th day from transplantation, we expect that engrafted T-cells will express the high-affinity LFA-1 within 14 days after transplantation. After that we expect that the expression of the high-affinity LFA-1 will decrease as anergy is induced. We anticipate that cells that express the high-affinity LFA-1 will be found in ICAM-rich tissues such as the liver, lung, and gut as those cells are more adhesive to ICAMs. As AL-57-NP functions as a multivalent antibody, we expect that it will show a greater binding to the active LFA-1 than bivalent free AL-57.

Xenogenic response in NOD/Lt-scid IL2rγ^{null} mice might be less robust than that in NOD/Lt-scid and NOD/Lt-scid B2mnull mice. Should we be unable to detect any binding of AL-57, AL-57-NP, or AL-57-PF in any time points, we will use CBRLFA-1/2 to enforce activation of LFA-1 in vivo. CBRLFA-1/2, which directly acts on the extracellular part of LFA-1, will activate LFA-1 on the cell surface regardless of levels of xenogenic activation and the induction of anergy. mAb TS2/4 will be used as a reference to CBRLFA-1/2. Enforced activation of LFA-1 by injection of CBRLFA-1/2 will be monitored by examining exposure of AL-57 and KIM127 epitopes. The amount of CBRLFA-1/2 (50, 100, 250 μg/mice) will be titrated so that the high-affinity LFA-1 will be induced while mice will be healthy with no signs of fatal effects.

Should we be unable to detect binding of AL-57 but able to detect binding of KIM127, we will consider developing KIM127-NP. (Please note that as previously mentioned in example 2.1.1, activation of LFA-1 defined by KIM127 is less stringent than that by AL-57.) We obtained the KIM127 hybridoma from ATCC. KIM127-NP will be created and characterized as described above for AL-57-NP. As KIM127 is not function-blocking but favors the active conformation of LFA-1 [104], we anticipate that KIM127-NP will enhance LFA-1-mediated binding to ICAM-1 and signaling. This activating effect of KIM127 may be, at least in part, neutralized by including siRNA to the αL subunit of LFA-1 to knock down the expression of LFA-1 selectively in activated cells. We will study integrin activation as described in example 2.1.3. Because of KIM127's activating property, we assign a higher priority to AL-57-NP and consider KIM127-NP as a back-up.

Should human cells in NOD/Lt-scid IL2rγ^{null}-hu-PBL continue to express active LFA-1, we will consider use of NOD/Lt-scid IL2rγ^{null} mice transplanted with human CD34+HSC [122, 123]. Reconstitution of human hematopoietic cells via transplantation of human HSC in NOD/Lt-scid IL2rγ^{null} mice elicits little xenogenic response. Therefore, resulting humanized mice carry human hematopoietic cells that usually do not show activated phenotypes and will serve as a comparison animal model, in which leukocytes express latent human LFA-1. We will confirm in the mice that LFA-1 is predominantly in the low-affinity conformation by IFC as described for NOD/Lt-scid IL2rγ^{null}-hu-PBL. We are aware that not only lymphocytes but also macrophages and granulocytes will be reconstituted [122], providing us with more clinically relevant but more complicated humanized mouse model.

### Example 2.2.2

### Biodistribution and Pharmacokinetics of AL-57-Nanoparticles

Rationale.

Delivery of chemotherapy agents by immuno-liposomes has been studied in SCID mice that carrying human cancer cells [124-126]) These studies suggested that the biodistribution is significantly varied depending on size and composition of liposomes, surface modification for long-circulation, and types of antibodies. Unlike liposomes, the biodistribution and pharmacokinetics of antibody-protamine fusion proteins remains to be unexplored. It is of great importance to investigate and compare biodistribution of AL-57-NP and AL-57-PF as well as AL-57- and TS1/22-directed delivery in immuno-nanoparticles and protamine-fusion protein. We will study the biodistribution of the delivery vehicles in NOD/Lt-scid IL2rγ^{null}-hu-PBL before and after the induction of anergy.

Biodistribution

Biodistribution of the immuno-nanoparticles will be studied with 14C-cholesterol as described [124]. Blood samples will be drawn at designated time after administration of radio-isotope labeled particles (5 min, 30 min, 1 hr, 3 hr, 6 hr, 12 hr, 24 hr, 48 hr, and 72 hr). In some experiments, mice will be sacrificed at designated time points (1, 24, 48 hr after injection) and organs such as the liver, lung, spleen, kidneys, and gut will be harvested and homogenized and lysed with a Polytron homogenizer (Brinkman Instruments, Mississauga, Ontario). Tissue lysates will be assayed for radioactivity by liquid scintillation counting with a Beckman LS 6500 liquid scintillation counter. Values will be corrected for plasma levels. Biodistribution of protamine fusion proteins will be studied using 32P-labeling as described [127].

Anticipated Results & Potential Pitfalls/Alternative Approaches

We expect that AL-57-NP and -PF will circulate longer than TS1/22-NP and -PF, as AL-57-NP and -PF will not be cleared from circulation by binding to latent LFA-1. We anticipate some accumulation of nanoparticles in the liver and spleen as previously shown in hyaluronan-coated liposomes [76]. Should substantial uptake by the liver that compromises long circulation be observed, we will consider using other methods of steric protection such as PEG attached to the surface of liposomes [43, 44]. The Fc-portion of IgG AL-57 may induce rapid clearance through Fc receptors; we will consider replacing IgG AL-57 with Fab AL-57.

Free antibody-protamine fusion protein (30.5 kDa), which is smaller than siRNA-complexed protein by 40 to 50 kDa (corresponding to 6 to 7 siRNA molecules), will be more subjected to renal clearance. Thus, we will also examine biodistribution and pharmacokinetics of 32P-labeled protamine fusion protein complexed with control siRNA. Should we decide to use NOD/Lt-scid IL2rγ^{null}-hu-PBL treated with CBRLFA-1/2 and/or NOD/Lt-scid IL2rγ^{null}-hu-HSC, we will study biodistributions in those mice as well.

### Example 2.2.3

### siRNA Delivery and Gene-Silencing by AL-57-Nano-Particles In Vivo

Rationale.

We will investigate the feasibility of siRNA delivery and gene-silencing by AL-57-NP and -PF in vivo to NOD/Lt-scid IL2rγ^{null}-hu-PBL. Many steps are needed to determine whether active leukocyte-selective delivery is possible. First we will confirm binding of AL-57-NP and -PF ex vivo as mentioned above. Second, we will examine the in vivo binding of Cy3-labeled vehicles. Third, we will study the delivery of Cy3-siRNAs formulated in AL-57-NP or -PF. Finally, we will advance to the investigations of in vivo gene silencing with Ku70-siRNAs incorporated into the delivery carriers.

Binding of Cy3-Labeled Immuno-Nanoparticles In Vivo

After confirming ex vivo that at least subsets of engrafted human lymphocytes are positive for binding of free AL-57 as well as Cy3-AL-57-NP in D.3.1, we will study in vivo binding to LFA-1 of the immuno-particles (AL-57-TS1/22-, and IgG-NPs). We will use Cy3-immuno-nanoparticles prepared. Cy3-immuno-nanoparticles will be injected via the tail vein to NOD/Lt-scid IL2rγ^{null}-hu-PBL before and after the induction of anergy. PBMC and cells from tissues mentioned above will be studied at designated time points (1, 24, 48, 72 hr after injection). Cells will be stained with mAbs TS2/4-FTIC and KIM127-PerCP to identify the presence of Cy3-AL-57-NP in the high-affinity human LFA-1-expressing cells (KIM127high TS2/4+ cells).

In some experiments, histological analysis will be done in the organs listed above. Neither AL-57 nor KIM127 is established for tissue staining, making it difficult to study LFA-1 activation in histology slides. Therefore, in histology we will focus on studying uptake of Cy3-AL-57-NP by RES. We will examine whether or not Cy3-AL-57-NP will be associated with human LFA-1-negative cells (TS2/4-). In the case that we observe substantial association of Cy3-AL-57-NP with FITC-TS2/4-negative cells, we will determine uptake of particles by RES. We will study association of Cy3-particles with mouse macrophages (anti-Mac-1+-FITC) and endothelial cells (anti-CD146+-FITC).

We will investigate in vivo binding of the protamine-fusion proteins (AL-57-, TS1/22-, and ML39-PFs) as mentioned above for the immuno-nanoparticles. We will use Cy3-labeled protamine fusion proteins complexed with control luciferase-siRNA.

Delivery of Cy3-siRNAs

After confirming in vivo binding of AL-57-NP and -PF to high-affinity LFA-1-expressing cells, we will study delivery of Cy3-siRNA. We will administer 10 nmol Cy3-siRNA encapsulated in the immuno-nanoparticles to NOD/Lt-scid IL2rγ^{null}-hu-PBL mice. We will start with 10 nmol of siRNA, as a similar amount was used in vivo for treating implanted tumor cells with delivery by protamine fusion protein [18]. We will study the presence of Cy3-positive cells as mentioned above by IFC and histology. We will also study Cy3-siRNA delivery by the protamine fusion proteins as described for the immuno-nanoparticles.

Gene-Silencing by Ku70-siRNA

After confirming in vivo binding of the particles and Cy3-siRNA delivery, we will investigate gene silencing with Ku70-siRNA. We will formulate Ku70-siRNAs in the immuno-nanoparticles and confirm the ability of in vitro silencing in every batch as described (FIG. 6). We will intravenously administer 10 nmol Ku70-siRNA in the nanoparticles to NOD/Lt-scid IL2rγ^{null}-hu-PBL mice before and after the induction of anergy. PBMC and mononuclear cells from the organs will be isolated as described at designated time points (24, 48, 72) hr after injection and examined by IFC. After staining of cell surface molecules with FTIC- and/or PerCP-labeled mAbs, cells will be fixed, permealized, and stained with PE-labeled mAb to Ku70. The Ku70 expression in high-affinity (KIM127high TS2/4+) and low-affinity (KIM127low TS2/4+) LFA-1-expressing cells will be compared in mice treated with AL-57-, TS1/22-, and IgG-NPs. We will also study gene silencing with Ku70-siRNA by the protamine fusion proteins as described for the immuno-nanoparticles.

Anticipated Results & Potential Pitfalls/Alternative Approaches.

The significance of the results obtained in this section is two-fold: 1) a side-by-side comparison of the active LFA-1 selective and non-selective delivery, and 2) direct comparison of two novel delivery vehicles, the scFv-protamine fusion protein and the immuno-nanoparticles, targeting the same molecule. As supported by preliminary data in vitro, we expect that Cy3-siRNA delivery and Ku70 silencing by AL-57-NP and -PF will be selective to KIM127high cells that express the high-affinity LFA-1, whereas those by TS1/22-NP and -PF will be towards TS2/4+ cells regardless of KIM127 expression. Although we are aware of many pitfalls in achieving efficient in vivo siRNA delivery and gene silencing, our step-by-step approach outlined above will allow us to identify and manage specific problems along the way. As mentioned in D.3.2, entrapment of the immuno-nanoparticles in the spleen, lung, and liver may interfere with siRNA delivery and gene silencing. Thus, we expect that the delivery by the protamine fusion proteins may provide us with better silencing because of less uptake by RES. However, unlike liposome-incorporated siRNAs that are highly protected from inactivation and degradation from external environments, the protamine fusion protein-complexed siRNAs might be more susceptible to degradation in vivo. We will consider the option of using chemically modified siRNAs that are less vulnerable to the degradation. However, chemically modified siRNAs might reduce a silencing efficacy in the cytoplasm [28]. Should we be able to observe little silencing, we will increase the amount of particles and/or siRNA. We are aware of potential inflammatory responses elicited by interferon responses and/or integrin-signaling. We will examine serum IFN-γ, IL-6, and TNF-α with ELISA (BD Bioscience). Should injection of the immuno-nanoparticles harm mice, we will titrate down the amount of particles and/or siRNA. Alternatively, we will consider using the protamine fusion protein, which we expect will induce less inflammatory response, if any.

### Discussion

### Integrin LFA-1 as Drug Delivery Target

We show here the use of the integrin LFA-1 as a drug delivery target to leukocytes for anti-inflammatory therapy. Our strategy is similar to the use of integrin αᵥβ₃ as a cancer drug delivery mechanism [55, 56]. However, it differs in that our antibody selectively binds to the active conformation of the integrin, improving pharmacokinetic properties of our targeting system as well as manipulating only the aberrantly activated cells without perturbing immune homeostasis. As summarized below, there are multiple lines of evidence that validate the use of LFA-1 for a drug delivery target to leukocytes: First, LFA-1 is exclusively expressed in all subsets of leukocytes. In particular, the expression is high in lymphocytes. This unique expression of LFA-1 to leukocytes makes this integrin appropriate for leukocyte-specific targeting. Second, LFA-1 is constitutively internalized and recycled in leukocytes. Regulated internalization of LFA-1 is implicated in facilitating detachment for efficient directional cell migration [54]. ICAM-1-derived peptides [57] as well as antibodies to the ligand-binding domain of LFA-1 [24] have been shown to induce internalization. Thus, LFA-1 recycling supports internalization of bound antibodies and peptides, a requisite for efficient drug delivery. Third, by converting to the high-affinity conformation (which exposes distinct epitopes), LFA-1 provides a targetable marker highly specific for activated leukocytes. We have shown by crystallography that the ligand binding domain of LFA-1, termed an inserted (I) domain, undergoes conformational changes from the low-affinity, closed form to the high-affinity, open form with a progressive 10,000-fold increase in affinity in the activated state [58]. The activity of LFA-1 is dynamically regulated on the cell surface. LFA-1 is usually in the low-affinity non-adhesive form in näive cells, and converted through the conformational changes to the high-affinity adhesive form upon leukocyte activation [59, 60]. Therefore, targeting the high-affinity form of LFA-1 (e.g. by mAb AL-57 that preferentially binds to the high-affinity LFA-1) will enable drug delivery selective for activated and adhesive leukocytes. As LFA-1-mediated internalization and lysosomal degradation are proposed to be a major pathway to clear LFA-1 antibodies from circulation [24], the selective targeting to the active LFA-1 will improve delivery pharmacokinetics by eliminating unnecessary mAb binding. Ideally, selective targeting of the activated and adhesive leukocytes will be sufficient for suppressing inflammatory tissue injury caused by leukocyte accumulation. Furthermore, by leaving näive cells untouched, selective targeting will be advantageous in reducing iatrogenic immune-defects. Fourth, many antibodies to LFA-1 including AL-57 block leukocyte adhesion (FIG. 1D). Targeted drug delivery using function blocking LFA-1 antibodies may produce additive or synergetic effects of silencing of proinflammatory molecules with inhibition of LFA-1-mediated cell adhesion. As blocking LFA-1 alone is not sufficient to suppress inflammation in certain disease models [61], the combination of LFA-1 blocking antibodies with gene silencing will be a novel therapeutic approach.

siRNA and Delivery

RNAi is an evolutionally conserved gene-silencing phenomenon. The discovery of the effective operation of RNAi in mammalian cells [1,2] has revolutionized biomedical research and RNAi has progressed from a valuable research tool to a potentially powerful therapeutic approach for treating cancer, virus infections, degenerative diseases, and inflammation [22, 23, 25, 26]. RNAi can be achieved either by expressing siRNA precursors such as short hairpin RNA (shRNA) with viral vectors or by directly incorporating synthetic siRNAs into the cytoplasm of cells. The use of synthetic siRNAs as small-molecule drugs for gene silencing avoids clinical safety concerns associated with viral vectors.

Local delivery of siRNAs using cationic lipids and polymer reagents used for transfection in vitro has been effective at the mucosal surface such as the lung and vagina. siRNAs complexed with polyethylenimine (PEI) or Oligofectamine™ (Invitrogen Corp., Carlsbad, Calif.) were locally injected to suppress viral infection in the lung [15, 16] and vagina [17]. Successful local delivery of siRNAs to other tissues via several different carriers have also been reported including the eye [29, 30], subcutaneous tumor [31, 32], and the central nerve system [33-35].

Cell-type- and tissue-specific delivery of siRNAs is ideal for maximizing the efficacy of gene silencing while reducing unwanted collateral damage to benign tissue. Selective delivery to target cells and tissues by systemic administration is an appealing approach that is widely applicable for treating many pathological conditions. Antibody and ligand-mediated delivery of siRNAs via cell surface receptors has emerged as a promising therapeutic approach. A modified cationic polymer PEI with an Arg-Gly-Asp (RGD) peptide ligand attached was used to deliver siRNA to tumor vasculature that expresses RGD-binding αᵥ integrins [20]. Liposomes displaying mAb to transferrin receptor were intravenously injected for delivering siRNA against EGF-receptor to glioma implanted in the brain [42]. More recently, an antibody-protamine fusion protein has been used for the tissue-specific delivery of siRNAs in vivo (WO 2006/023491). Using an anti-gp160 (a HIV envelop glycoprotein) antibody-protamine fusion protein, a cocktail of siRNAs to c-myc, MDM2, and VEGF was delivered to mice carrying subcutaneously B16 melanoma cells engineered to express gp160. The siRNA treatment significantly reduced the size of the tumor, forming a foundation to the systemic, cell-type specific, antibody-mediated siRNA delivery [18].

### Targeted Delivery of Liposomes for Anti-Inflammatory Therapies

Liposomes are probably the most widely used drug carrier system with many attractive biological properties [43, 44]. Most liposomes consist of non-toxic and biocompatible neutral lipids; liposomes can entrap hydrophilic agents in their internal water compartment and hydrophobic ones in the membrane; liposome-incorporated agents are protected from inactivation and degradation from external environments; liposomes have a capacity to deliver their cargo into cells; and surface properties of liposomes can be modified with specific antibodies and ligands.

A drawback in the early stage of the systemic use of liposomes is the fast elimination from the blood and capture by cells of the reticulo-endothelial system (RES) [43]. Coating the surface of liposomes with an inert, biocompatible polymer polyethylene glycol (PEG) slows down liposome recognition by opsonins and subsequent clearance, thereby generating long-circulating liposomes [45]. The glycosaminoglycan hyaluronan used in the examples below also generates long-circulating liposomes [46] and has the additional advantage of serving as a cryoprotectant [47].

Various monoclonal antibodies and ligands have been used to direct liposomes to specific targets, including cell surface molecules over-expressed in cancer cells such as HER2, folate receptor, transferring receptor, EGF-receptor, and αᵥ integrins [43]. In addition to delivering chemotherapeutic drugs to tumor cells, applications of immuno-liposomes in the treatment of inflammatory diseases have been investigated, in which proinflammatory molecules were targeted [44]. E-selectin-targeted liposomes containing dexamethasone were examined in murine delayed-type hypersensitivity model and showed increased uptake by endothelial cells at sites of inflammation [48]. Uptake of Immuno-liposomes targeting VCAM-1 by TNF-α-activated HUVEC was reported [49]. A drug delivery system targeting activated leukocytes has been explored as a novel anti-inflammatory therapy [44, 50]. Immuno-liposomes targeting CD134 (OX40) expressed on activated lymphocytes were used to deliver a cytostatic drug 5′-fluorodeoxyuridine and showed amelioration of adjuvant arthritis [50]. However, CD134 does not support internalization of the immune-liposomes and is therefore not ideal for delivery into cells [50]. The receptor-mediated internalization exhibited in other molecules on leukocytes such as CD2, CD3, CD5, and integrins [51-54] makes these cell surface proteins preferable targets for a siRNA delivery to leukocytes.

### REFERENCES FOR EXAMPLES 1-2, BACKGROUND AND DETAILED DESCRIPTION

1. Hannon, G. J. RNA interference. Nature 418(6894), 244-51. (2002).
2. Sharp, P. A. RNA interference—2001. Genes Dev 15(5), 485-90. (2001).
3. Tijsterman, M., R. F. Ketting, and R. H. Plasterk. The genetics of RNA silencing. Annu Rev Genet 36, 489-519. (2002).
4. Zamore, P. D. RNA interference: listening to the sound of silence. Nat Struct Biol 8(9), 746-50. (2001).
5. Ketting, R. F., T. H. Haverkamp, H. G. van Luenen, and R. H. Plasterk. Mut-7 of C. elegans, required for transposon silencing and RNA interference, is a homolog of Werner syndrome helicase and RNaseD. Cell 99(2), 133-41. (1999).
6. Tabara, H., M. Sarkissian, W. G. Kelly, J. Fleenor, A. Grishok, L. Timmons, A. Fire, and C. C. Mello. The rde-1 gene, RNA interference, and transposon silencing in C. elegans. Cell 99(2), 123-32. (1999).
7. Jones, A. R. and T. Schedl. Mutations in gld-1, a female germ cell-specific tumor suppressor gene in Caenorhabditis elegans, affect a conserved domain also found in Src-associated protein Sam68. Genes Dev 9(12), 1491-504. (1995).
8. Gaudet, J., I. VanderElst, and A. M. Spence. Post-transcriptional regulation of sex determination in Caenorhabditis elegans: widespread expression of the sex-determining gene fem-1 in both sexes. Mol Biol Cell 7(7), 1107-21. (1996).
9. Pal-Bhadra, M., U. Bhadra, and J. A. Birchler. Cosuppression of nonhomologous transgenes in Drosophila involves mutually related endogenous sequences. Cell 99(1), 35-46. (1999).
10. Waterhouse, P. M., M. B. Wang, and T. Lough. Gene silencing as an adaptive defense against viruses. Nature 411(6839), 834-42. (2001).
11. Bernstein, E., A. A. Caudy, S. M. Hammond, and G. J. Hannon. Role for a bidentate ribonuclease in the initiation step of RNA interference. Nature 409(6818), 363-6. (2001).
12. Elbashir, S. M., J. Harborth, W. Lendeckel, A. Yalcin, K. Weber, and T. Tuschl. Duplexes of 21-nucleotide RNAs mediate RNA interference in cultured mammalian cells. Nature 411(6836), 494-8. (2001).
13. Hammond, S. M., E. Bernstein, D. Beach, and G. J. Hannon. An RNA-directed nuclease mediates post-transcriptional gene silencing in Drosophila cells. Nature 404(6775), 293-6. (2000).
14. Elbashir, S. M., W. Lendeckel, and T. Tuschl. RNA interference is mediated by 21- and 22-nucleotide RNAs. Genes Dev 15(2), 188-200. (2001).
15. Bitko, V., A. Musiyenko, O. Shulyayeva, and S. Barik. Inhibition of respiratory viruses by nasally administered siRNA. Nat Med 11(1), 50-5. (2005).
16. Tompkins, S. M., C. Y. Lo, T. M. Tumpey, and S. L. Epstein. Protection against lethal influenza virus challenge by RNA interference in vivo. Proc Natl Acad Sci USA 101(23), 8682-6. (2004).
17. Palliser, D., D. Chowdhury, Q. Y. Wang, S. J. Lee, R. T. Bronson, D. M. Knipe, and J. Lieberman. An siRNA-based microbicide protects mice from lethal herpes simplex virus 2 infection. Nature. (2005).
18. Song, E., P. Zhu, S. K. Lee, D. Chowdhury, S. Kussman, D. M. Dykxhoorn, Y. Feng, D. Palliser, D. B. Weiner, P. Shankar, W. A. Marasco, and J. Lieberman. Antibody mediated in vivo delivery of small interfering RNAs via cell-surface receptors. Nat Biotechnol 23(6), 709-17. (2005).
19. Zhang, F., Y. Wu, Q. Ma, D. Hoppensteadt, J. Fareed, and R. J. Linhardt. Studies on the effect of calcium in interactions between heparin and heparin cofactor II using surface plasmon resonance. Clin. Appl. Thromb. Hemost. 10, 249-257. (2004).
20. Schiffeters, R. M., A. Ansari, J. Xu, Q. Zhou, Q. Tang, G. Storm, G. Molema, P. Y. Lu, P. V. Scaria, and M. C. Woodle. Cancer siRNA therapy by tumor selective delivery with ligand-targeted sterically stabilized nanoparticle. Nucleic Acids Res 32(19), e149. (2004).
21. Song, E., S. K. Lee, J. Wang, N. Ince, N. Ouyang, J. Min, J. Chen, P. Shankar, and J. Lieberman. RNA interference targeting Fas protects mice from fulminant hepatitis. Nat Med 9(3), 347-51. (2003).
22. Dykxhoorn, D. M. and J. Lieberman. The silent revolution: RNA interference as basic biology, research tool, and therapeutic. Annu Rev Med 56, 401-23. (2005).
23. Shankar, P., N. Manjunath, and J. Lieberman. The prospect of silencing disease using RNA interference. Jama 293(11), 1367-73. (2005).
24. Coffey, G. P., E. Stefanich, S. Palmieri, R. Eckert, J. Padilla-Eagar, P. J. Fielder, and S. Pippig. In vitro internalization, intracellular transport, and clearance of an anti-CD11a antibody (Raptiva) by human T-cells. J Pharmacol Exp Ther 310(3), 896-904. (2004).
25. Sledz, C. A. and B. R. Williams. RNA interference in biology and disease. Blood 106(3), 787-94. (2005).
26. Leung, D. W. and M. K. Rosen. The nucleotide switch in Cdc42 modulates coupling between the GTPase-binding and allosteric equilibria of Wiskott-Aldrich syndrome protein. Proc. Natl. Acad. Sci. USA 102, 5685-5690. (2005).
27. Stewart, S. A., D. M. Dykxhoorn, D. Palliser, H. Mizuno, E. Y. Yu, D. S. An, D. M. Sabatini, I. S. Chen, W. C. Hahn, P. A. Sharp, R. A. Weinberg, and C. D. Novina. Lentivirus-delivered stable gene silencing by RNAi in primary cells. Rna 9(4), 493-501. (2003).
28. Soutschek, J., A. Akinc, B. Bramlage, K. Charisse, R. Constien, M. Donoghue, S. Elbashir, A. Geick, P. Hadwiger, J. Harborth, M. John, V. Kesavan, G. Lavine, R. K. Pandey, T. Racie, K. G. Rajeev, I. Rohl, I. Toudjarska, G. Wang, S. Wuschko, D. Bumcrot, V. Koteliansky, S. Limmer, M. Manoharan, and H. P. Vornlocher. Therapeutic silencing of an endogenous gene by systemic administration of modified siRNAs. Nature 432(7014), 173-8. (2004).
29. Reich, S. J., J. Fosnot, A. Kuroki, W. Tang, X. Yang, A. M. Maguire, J. Bennett, and M. J. Tolentino. Small interfering RNA (siRNA) targeting VEGF effectively inhibits ocular neovascularization in a mouse model. Mol V is 9, 210-6. (2003).
30. Kim, B., Q. Tang, P. S. Biswas, J. Xu, R. M. Schiffelers, F. Y. Xie, A. M. Ansari, P. V. Scaria, M. C. Woodle, P. Lu, and B. T. Rouse. Inhibition of ocular angiogenesis by siRNA targeting vascular endothelial growth factor pathway genes: therapeutic strategy for herpetic stromal keratitis. Am J Pathol 165(6), 2177-85. (2004).
31. Takei, Y., K. Kadomatsu, Y. Yuzawa, S. Matsuo, and T. Muramatsu. A small interfering RNA targeting vascular endothelial growth factor as cancer therapeutics. Cancer Res 64(10), 3365-70. (2004).
32. Minakuchi, Y., F. Takeshita, N. Kosaka, H. Sasaki, Y. Yamamoto, M. Kouno, K. Honma, S. Nagahara, K. Hanai, A. Sano, T. Kato, M. Terada, and T. Ochiya. Atelocollagen-mediated synthetic small interfering RNA delivery for effective gene silencing in vitro and in vivo. Nucleic Acids Res 32(13), e109. (2004).
33. Xia, H., Q. Mao, S. L. Eliason, S. Q. Harper, I. H. Martins, H. T. Orr, H. L. Paulson, L. Yang, R. M. Kotin, and B. L. Davidson. RNAi suppresses polyglutamine-induced neurodegeneration in a model of spinocerebellar ataxia. Nat Med 10(8), 816-20. (2004).
34. Raoul, C., T. Abbas-Terki, J. C. Bensadoun, S. Guillot, G. Haase, J. Szulc, C. E. Henderson, and P. Aebischer. Lentiviral-mediated silencing of SOD1 through RNA interference retards disease onset and progression in a mouse model of ALS. Nat Med 11(4), 423-8. (2005).
35. Ralph, G. S., P. A. Radcliffe, D. M. Day, J. M. Carthy, M. A. Leroux, D. C. Lee, L. F. Wong, L. G. Bilsiand, L. Greensmith, S. M. Kingsman, K. A. Mitrophanous, N. D. Mazarakis, and M. Azzouz. Silencing mutant SOD1 using RNAi protects against neurodegeneration and extends survival in an ALS model. Nat Med 11(4), 429-33. (2005).
36. Miyawaki-Shimizu, K., D. Predescu, J. Shimizu, M. Broman, S. Predescu, and A. B. Malik. siRNA-Induced Caveolin-1 Knock-Down in Mice Increases Lung Vascular Permeability via the Junctional Pathway. Am J Physiol Lung Cell Mol Physiol. (2005).
37. McCaffrey, A. P., L. Meuse, T. T. Pham, D. S. Conklin, G. J. Hannon, and M. A. Kay. RNA interference in adult mice. Nature 418(6893), 38-9. (2002).
38. Wesche-Soldato, D. E., C. S. Chung, J. Lomas-Neira, L. A. Doughty, S. H. Gregory, and A. Ayala. In vivo delivery of caspase-8 or Fas siRNA improves the survival of septic mice. Blood 106(7), 2295-301. (2005).
39. Zender, L., S. Hutker, C. Liedtke, H. L. Tillmann, S. Zender, B. Mundt, M. Waltemathe, T. Gosling, P. Flemming, N. P. Malek, C. Trautwein, M. P. Manns, F. Kuhnel, and S. Kubicka. Caspase 8 small interfering RNA prevents acute liver failure in mice. Proc Natl Acad Sci USA 100(13), 7797-802. (2003).
40. Morrissey, D. V., K. Blanchard, L. Shaw, K. Jensen, J. A. Lockridge, B. Dickinson, J. A. McSwiggen, C. Vargeese, K. Bowman, C. S. Shaffer, B. A. Polisky, and S. Zinnen. Activity of stabilized short interfering RNA in a mouse model of hepatitis B virus replication. Hepatology 41(6), 1349-56. (2005).
41. Morrissey, D. V., J. A. Lockridge, L. Shaw, K. Blanchard, K. Jensen, W. Breen, K. Hartsough, L. Machemer, S. Radka, V. Jadhav, N. Vaish, S. Zinnen, C. Vargeese, K. Bowman, C. S. Shaffer, L. B. Jeffs, A. Judge, I. MacLachlan, and B. Polisky. Potent and persistent in vivo anti-HBV activity of chemically modified siRNAs. Nat Biotechnol 23(8), 1002-7. (2005).
42. Zhang, Y., Y. F. Zhang, J. Bryant, A. Charles, R. J. Boado, and W. M. Pardridge. Intravenous RNA interference gene therapy targeting the human epidermal growth factor receptor prolongs survival in intracranial brain cancer. Clin Cancer Res 10(11), 3667-77. (2004).
43. Torchilin, V. P. Recent advances with liposomes as pharmaceutical carriers. Nat Rev Drug Discov 4(2), 145-60. (2005).
44. Metselaar, J. M. and G. Storm. Liposomes in the treatment of inflammatory disorders. Expert Opin Drug Deliv 2(3), 465-76. (2005).
45. Maruyama, K. PEG-immunoliposome. Biosci Rep 22(2), 251-66. (2002).
46. Peer, D. and R. Margalit. Loading mitomycin C inside long circulating hyaluronan targeted nano-liposomes increases its antitumor activity in three mice tumor models. Int J Cancer 108(5), 780-9. (2004).
47. Peer, D., A. Florentin, and R. Margalit. Hyaluronan is a key component in cryoprotection and formulation of targeted unilamellar liposomes. Biochim Biophys Acta 1612(1), 76-82. (2003).
48. Everts, M., G. A. Koning, R. J. Kok, S. A. Asgeirsdottir, D. Vestweber, D. K. Meijer, G. Storm, and G. Molema. In vitro cellular handling and in vivo targeting of E-selectin-directed immunoconjugates and immunoliposomes used for drug delivery to inflamed endothelium. Pharm Res 20(1), 64-72. (2003).
49. Voinea, M., 1. Manduteanu, E. Dragomir, M. Capraru, and M. Simionescu. Immunoliposomes directed toward VCAM-1 interact specifically with activated endothelial cells—a potential tool for specific drug delivery. Pharm Res 22(11), 1906-17. (2005).
50. Boot, E. P., G. A. Koning, G. Storm, J. P. Wagenaar-Hilbers, W. van Eden, L. A. Everse, and M. H. Wauben. CD134 as target for specific drug delivery to auto-aggressive CD4+ T cells in adjuvant arthritis. Arthritis Res Ther 7(3), R604-15. (2005).
51. Matthay, K. K., A. M. Abai, S. Cobb, K. Hong, D. Papahadjopoulos, and R. M. Straubinger. Role of ligand in antibody-directed endocytosis of liposomes by human T-leukemia cells. Cancer Res 49(17), 4879-86. (1989).
52. Dinauer, N., S. Balthasar, C. Weber, J. Kreuter, K. Langer, and H. von Briesen. Selective targeting of antibody-conjugated nanoparticles to leukemic cells and primary T-lymphocytes. Biomaterials 26(29), 5898-906. (2005).
53. Balthasar, S., K. Michaelis, N. Dinauer, H. von Briesen, J. Kreuter, and K. Langer. Preparation and characterisation of antibody modified gelatin nanoparticles as drug carrier system for uptake in lymphocytes. Biomaterials 26(15), 2723-32. (2005).
54. Fabbri, M., S. Di Meglio, M. C. Gagliani, E. Consonni, R. Molteni, J. R. Bender, C. Tacchetti, and R. Pardi. Dynamic Partitioning into Lipid Rafts Controls the Endo-Exocytic Cycle of the {alpha}L/{beta}2 Integrin, LFA-1, during Leukocyte Chemotaxis. Mol Biol Cell 16(12), 5793-803. (2005).
55. Hood, J. D., M. Bednarski, R. Frausto, S. Guccione, R. A. Reisfeld, R. Xiang, and D. A. Cheresh. Tumor regression by targeted gene delivery to the neovasculature. Science 296(5577), 2404-7. (2002).
56. Guccione, S., K. C. Li, and M. D. Bednarski. Molecular imaging and therapy directed at the neovasculature in pathologies. How imaging can be incorporated into vascular-targeted delivery systems to generate active therapeutic agents. IEEE Eng Med Biol Mag 23(5), 50-6. (2004).
57. Anderson, M. E. and T. J. Siahaan. Mechanism of binding and internalization of ICAM-1-derived cyclic peptides by LFA-1 on the surface of T cells: a potential method for targeted drug delivery. Pharm Res 20(10), 1523-32. (2003).
58. Shimaoka, M., T. Xiao, J.-H. Liu, Y. Yang, Y. Dong, C.-D. Jun, A. McCormack, R. Zhang, A. Joachimiak, J. Takagi, J.-h. Wang, and T. A. Springer. Structures of the aL I domain and its complex with ICAM-1 reveal a shape-shifting pathway for integrin regulation. Cell 112, 99-111. (2003).
59. Carman, C. V. and T. A. Springer. Integrin avidity regulation: Are changes in affinity and conformation underemphasized? Curr. Opin. Cell Biol. 15, 547-556. (2003).
60. Kinashi, T. and K. Katagiri. Regulation of lymphocyte adhesion and migration by the small GTPase Rap1 and its effector molecule, RAPL. Immunol. Lett. 93, 1-5. (2004).
61. de Fougerolles, A. R., Integrins in immune and inflammatory diseases, in I Domains in Integrins, D. Gullberg, Editor. 2003, Plenum Publishers: Georgetown, Tex. p. 165-177.
62. Shimaoka, M., A. Salas, W. Yang, G. Weitz-Schmidt, and T. A. Springer. Small molecule integrin antagonists that bind to the b2 subunit I-like domain and activate signals in one direction and block them in another. Immunity 19, 391402. (2003).
63. Yusuf-Makagiansar, H., M. E. Anderson, T. V. Yakovleva, J. S. Murray, and T. J. Siahaan. Inhibition of LFA-1/ICAM-1 and VLA-4/VCAM-1 as a therapeutic approach to inflammation and autoimmune diseases. Med. Res. Rev. 22, 146-167. (2002).
64. Giblin, P. A. and T. A. Kelly. Antagonists of b2 integrin-mediated cell adhesion. Annu. Rep. Med. Chem. 36, 181-190. (2001).
65. Liu, S., D. A. Calderwood, and M. H. Ginsberg. Integrin cytoplasmic domain-binding proteins. J. Cell Sci. 113, 3563-3571. (2000).
66. Tadokoro, S., S. J. Shattil, K. Eto, V. Tai, R. C. Liddington, J. M. de Pereda, M. H. Ginsberg, and D. A. Calderwood. Talin binding to integrin b tails: a final common step in integrin activation. Science 302, 103-106. (2003).
67. Shamri, R., V. Grabovsky, J. M. Gauguet, S. Feigelson, E. Manevich, W. Kolanus, M. K. Robinson, D. E. Staunton, U. H. von Andrian, and R. Alon. Lymphocyte arrest requires instantaneous induction of an extended LFA-1 conformation mediated by endothelium-bound chemokines. Nat. Immunol. 6, 497-506. (2005).
68. Kim, M., C. V. Carman, and T. A. Springer. Bidirectional transmembrane signaling by cytoplasmic domain separation in integrins. Science 301, 1720-1725. (2003).
69. Kinashi, T., M. Aker, M. Sokolovsky-Eisenberg, V. Grabovsky, C. Tanaka, R. Shamri, S. Feigelson, A. Etzioni, and R. Alon. LAD-III, a leukocyte adhesion deficiency syndrome associated with defective Rap1 activation and impaired stabilization of integrin bonds. Blood 103, 1033-1036. (2004).
70. Harlan, J. M. and R. K. Winn. Leukocyte-endothelial interactions: clinical trials of anti-adhesion therapy. Crit. Care Med. 30, S214-S219. (2002).
71. Popescu, F. D. Antisense- and RNA interference-based therapeutic strategies in allergy. J Cell Mol Med 9(4), 840-53. (2005).
72. Pinkenburg, O., J. Platz, C. Beisswenger, C. Vogelmeier, and R. Bals. Inhibition of NF-kappaB mediated inflammation by siRNA expressed by recombinant adeno-associated virus. J Virol Methods 120(1), 119-22. (2004).
73. Roshak, A. K., J. F. Callahan, and S. M. Blake. Small-molecule inhibitors of NF-kappaB for the treatment of inflammatory joint disease. Curr Opin Pharmacol 2(3), 316-21. (2002).
74. Shimaoka, M., C. Lu, R. Palframan, U. H. von Andrian, J. Takagi, and T. A. Springer. Reversibly locking a protein fold in an active conformation with a disulfide bond: integrin aL I domains with high affinity and antagonist activity in vivo. Proc. Natl. Acad. Sci. U.S.A. 98, 6009-6014. (2001).
75. Sato, A. K., D. J. Sexton, L. A. Morganelli, E. H. Cohen, Q. L. Wu, G. P. Conley, Z. Streltsova, S. W. Lee, M. Devlin, D. B. DeOliveira, J. Enright, R. B. Kent, C. R. Wescott, T. C. Ransohoff, A. C. Ley, and R. C. Ladner. Development of mammalian serum albumin affinity purification media by peptide phage display. Biotechnol. Prog. 18, 182-192. (2002).
76. Peer, D. and R. Margalit. Tumor-targeted hyaluronan nanoliposomes increase the antitumor activity of liposomal Doxorubicin in syngeneic and human xenograft mouse tumor models. Neoplasia 6(4), 343-53. (2004).
77. Peer, D. and R. Margalit. Physicochemical evaluation of a stability-driven approach to drug entrapment in regular and in surface-modified liposomes. Arch Biochem Biophys 383(2), 185-90. (2000).
78. Peer, D., Y. Dekel, D. Melikhov, and R. Margalit. Fluoxetine inhibits multidrug resistance extrusion pumps and enhances responses to chemotherapy in syngeneic and in human xenograft mouse tumor models. Cancer Res 64(20), 7562-9. (2004).
79. Peer, D. and R. Margalit. Fluoxetine and reversal of multidrug resistance. Cancer Lett. (2005).
80. Cattel, L., M. Ceruti, and F. Dosio. From conventional to stealth liposomes: a new Frontier in cancer chemotherapy. J Chemother 16 Suppl 4, 94-7. (2004).
81. Laverman, P., O. C. Boerman, W. J. G. Oyen, F. H. M. Corstens, and G. Storm. In vivo applications of PEG liposomes: unexpected observations. Crit Rev Ther Drug Carrier Syst 18(6), 551-66. (2001).
82. Torchilin, V. P., R. Rammohan, V. Weissig, and T. S. Levchenko. TAT peptide on the surface of liposomes affords their efficient intracellular delivery even at low temperature and in the presence of metabolic inhibitors. Proc Natl Acad Sci USA 98(15), 8786-91. (2001).
83. Spragg, D. D., D. R. Alford, R. Greferath, C. E. Larsen, K. D. Lee, G. C. Gurtner, M. I. Cybulsky, P. F. Tosi, C. Nicolau, and M. A. Gimbrone, Jr. Immunotargeting of liposomes to activated vascular endothelial cells: a strategy for site-selective delivery in the cardiovascular system. Proc Natl Acad Sci USA 94(16), 8795-800. (1997).
84. Novina, C. D., M. F. Murray, D. M. Dykxhoom, P. J. Beresford, J. Riess, S. K. Lee, R. G. Collman, J. Lieberman, P. Shankar, and P. A. Sharp. siRNA-directed inhibition of HIV-1 infection. Nat Med 8(7), 681-6. (2002).
85. Takeshita, F., Y. Minakuchi, S. Nagahara, K. Honma, H. Sasaki, K. Hirai, T. Teratani, N. Namatame, Y. Yamamoto, K. Hanai, T. Kato, A. Sano, and T. Ochiya. Efficient delivery of small interfering RNA to bone-metastatic tumors by using atelocollagen in vivo. Proc Natl Acad Sci USA 102(34), 12177-82. (2005).
86. Kinashi, T. and K. Katagiri. Regulation of immune cell adhesion and migration by regulator of adhesion and cell polarization enriched in lymphoid tissues. Immunology 116(2), 164-71. (2005).
87. Perez, O. D., D. Mitchell, G. C. Jager, S. South, C. Murriel, J. McBride, L. A. Herzenberg, S. Kinoshita, and G. P. Nolan. Leukocyte functional antigen 1 lowers T cell activation thresholds and signaling through cytohesin-1 and Jun-activating binding protein 1. Nat. Immunol. 4, 1083-1092. (2003).
88. Constantin, G., M. Majeed, C. Giagulli, L. Piccib, J. Y. Kim, E. C. Butcher, and C. Laudanna. Chemokines trigger immediate b2 integrin affinity and mobility changes: differential regulation and roles in lymphocyte arrest under flow. Immunity 13, 759-769. (2000).
89. Chirathawom, C., J. E. Kohlmeier, S. A. Tibbetts, L. M. Rumsey, M. A. Chan, and S. H. Benedict. Stimulation through intercellular adhesion molecule-1 provides a second signal for T cell activation. J. Immunol. 168, 5530-5537. (2002).
90. Smith, A., M. Bracke, B. Leitinger, J. C. Porter, and N. Hogg. LFA-1-induced T cell migration on ICAM-1 involves regulation of MLCK-mediated attachment and ROCK-dependent detachment. J. Cell Sci. Epub. (2003).
91. Smith, A., Y. R. Carrasco, P. Stanley, N. Kieffer, F. D. Batista, and N. Hogg. A talin-dependent LFA-1 focal zone is formed by rapidly migrating T lymphocytes. J. Cell Biol. 170, 141-151. (2005).
92. Li, X., P. Stuckert, I. Bosch, J. D. Marks, and W. A. Marasco. Single-chain antibody-mediated gene delivery into ErbB2-positive human breast cancer cells. Cancer Gene Ther 8(8), 555-65. (2001).
93. Wagar, E. J., M. A. Cromwell, L. D. Shultz, B. A. Woda, J. L. Sullivan, R. M. Hesselton, and D. L. Greiner. Regulation of human cell engraftment and development of EBV-related lymphoproliferative disorders in Hu-PBL-scid mice. J Immunol 165(1), 518-27. (2000).
94. Thomsen, M., H. Yacoub-Youssef, and B. Marcheix. Reconstitution of a human immune system in immunodeficient mice: models of human alloreaction in vivo. Tissue Antigens 66(2), 73-82. (2005).
95. Lehmann, P. V., O. S. Targoni, and T. G. Forsthuber. Shifting T-cell activation thresholds in autoimmunity and determinant spreading. Immunol Rev 164, 53-61. (1998).
96. Szabo, S. J., B. M. Sullivan, S. L. Peng, and L. H. Glimcher. Molecular mechanisms regulating Th1 immune responses. Annu Rev Immunol 21, 713-58. (2003).
97. Singh, V. K., S. Mehrotra, and S. S. Agarwal. The paradigm of Th1 and Th2 cytokines: its relevance to autoimmunity and allergy. Immunol Res 20(2), 147-61. (1999).
98. Gudjonsson, J. E., A. Johnston, H. Sigmundsdottir, and H. Valdimarsson. Immunopathogenic mechanisms in psoriasis. Clin Exp Immunol 135(1), 1-8. (2004).
99. Hsu, H. C., D. K. Scott, and J. D. Mountz. Impaired apoptosis and immune senescence—cause or effect? Immunol Rev 205, 130-46. (2005).
100. Mizuno, K., A. Yachie, S. Nagaoki, H. Wada, K. Okada, M. Kawachi, T. Toma, A. Konno, K. Ohta, Y. Kasahara, and S. Koizumi. Oligoclonal expansion of circulating and tissue-infiltrating CD8+ T cells with killer/effector phenotypes in juvenile dermatomyositis syndrome. Clin Exp Immunol 137(1), 187-94. (2004).
101. Duncan, S. R., C. Leonard, J. Theodore, M. Lega, R. E. Girgis, G. D. Rosen, and A. N. Theofilopoulos. Oligoclonal CD4(+) T cell expansions in lung transplant recipients with obliterative bronchiolitis. Am J Respir Crit Care Med 165(10), 143944. (2002).
102. Sakkas, L. I., B. Xu, C. M. Artlett, S. Lu, S. A. Jimenez, and C. D. Platsoucas. Oligoclonal T cell expansion in the skin of patients with systemic sclerosis. J Immunol 168(7), 3649-59. (2002).
103. Chen, W. and C. D. Howell. Oligoclonal expansion of T cell receptor V beta 2 and 3 cells in the livers of mice with graft-versus-host disease. Hepatology 35(1), 23-9. (2002).
104. Lu, C., M. Ferzly, J. Takagi, and T. A. Springer. Epitope mapping of antibodies to the C-terminal region of the integrin b2 subunit reveals regions that become exposed upon receptor activation. J. Immunol. 166(9), 5629-5637. (2001).
105. Beglova, N., S. C. Blacklow, J. Takagi, and T. A. Springer. Cysteine-rich module structure reveals a fulcrum for integrin rearrangement upon activation. Nat. Struct. Biol. 9, 282-287. (2002).
106. Mora, J. R., M. R. Bono, N. Manjunath, W. Weninger, L. L. Cavanagh, M. Rosemblatt, and U. H. Von Andrian. Selective imprinting of gut-homing T cells by Peyer's patch dendritic cells. Nature 424, 88-93. (2003).
107. Langerak, A. W., R. van Den Beemd, I. L. Wolvers-Tettero, P. P. Boor, E. G. van Lochem, H. Hooijkaas, and J. J. van Dongen. Molecular and flow cytometric analysis of the Vbeta repertoire for clonality assessment in mature TCRalphabeta T-cell proliferations. Blood 98(1), 165-73. (2001).
108. Vorup-Jensen, T., T. T. Waldron, N. Astrof, M. Shimaoka, and T. A. Springer. The electrostatic switch mechanism in metal ion-dependent ligand binding by integrin I domains: studies by isothermal calorimetry and surface plasmon resonance. in preparation. (2005).
109. Harlan, J. M., R. K. Winn, N. B. Vedder, C. M. Doerschuk, and C. L. Rice, In vivo models of leukocyte adherence to endothelium, in Adhesion: Its Role in Inflammatory Disease, J. R. Harlan and D. Liu, Editors. 1992, W.H. Freeman & Company: New York. p. 117-150.
110. Hornung, V., M. Guenthner-Biller, C. Bourquin, A. Ablasser, M. Schlee, S. Uematsu, A. Noronha, M. Manoharan, S. Akira, A. de Fougerolles, S. Endres, and G. Hartmann. Sequence-specific potent induction of IFN-alpha by short interfering RNA in plasmacytoid dendritic cells through TLR7. Nat Med 11(3), 263-70. (2005).
111. Sioud, M. Induction of inflammatory cytokines and interferon responses by double-stranded and single-stranded siRNAs is sequence-dependent and requires endosomal localization. J Mol Biol 348(5), 1079-90. (2005).
112. Judge, A. D., V. Sood, J. R. Shaw, D. Fang, K. McClintock, and I. MacLachlan. Sequence-dependent stimulation of the mammalian innate immune response by synthetic siRNA. Nat Biotechnol 23(4), 457-62. (2005).
113. Ma, Z., J. Li, F. He, A. Wilson, B. Pitt, and S. Li. Cationic lipids enhance siRNA-mediated interferon response in mice. Biochem Biophys Res Commun 330(3), 755-9. (2005).
114. Fedorov, Y., A. King, E. Anderson, J. Karpilow, D. Ilsley, W. Marshall, and A. Khvorova Different delivery methods-different expression profiles. Nat Methods 2(4), 241. (2005).
115. Rossi, J. J. Receptor-targeted siRNAs. Nat Biotechnol 23(6), 6824. (2005).
116. Walzog, B., S. Offermanns, A. Zakrzewicz, P. Gaehtgens, and K. Ley. Beta2 integrins mediate protein tyrosine phosphorylation in human neutrophils. J Leukoc Biol 59(5), 747-53. (1996).
117. Kim, M., C. V. Carman, W. Yang, A. Salas, and T. A. Springer. The primacy of affinity over clustering in regulation of adhesiveness of the integrin aLb2. J. Cell Biol. 167, 1241-1253. (2004).
118. Kruisbeek, A. M., E. Shevach, and A. M. Thornton, Proliferative assays for T cell function, in Current Protocols in Immunology, J. E. Coligan, et al., Editors. 2003, John Wiley & Sons, Inc.
119. Lu, C., M. Shimaoka, A. Salas, and T. A. Springer. The binding sites for competitive antagonistic, allosteric antagonistic, and agonistic antibodies to the I domain of integrin LFA-1. J. Immunol. 173, 3972-3978. (2004).
120. Dustin, M. L. and T. A. Springer. Role of lymphocyte adhesion receptors in transient interactions and cell locomotion. Annu. Rev. Immunol. 9, 27-66. (1991).
121. Jin, M., G. Song, Y.-S. Kim, N. Astrof, M. Shimaoka, D. Wittrup, and T. A. Springer. Directed evolution to probe protein allestery: integrin I domains of unprecedented affinity. Proc Natl Acad Sci USA in press (2006).
122. Shultz, L. D., B. L. Lyons, L. M. Burzenski, B. Gott, X. Chen, S. Chaleff, M. Kotb, S. D. Gillies, M. King, J. Mangada, D. L. Greiner, and R. Handgretinger. Human lymphoid and myeloid cell development in NOD/LtSz-scid IL2R gamma null mice engrafted with mobilized human hemopoietic stem cells. J Immunol 174(10), 6477-89. (2005).
123. Ishikawa, F., M. Yasukawa, B. Lyons, S. Yoshida, T. Miyamoto, G. Yoshimoto, T. Watanabe, K. Akashi, L. D. Shultz, and M. Harada. Development of functional human blood and immune systems in NOD/SCID/IL2 receptor {gamma} chain(null) mice. Blood 106(5), 1565-73. (2005).
124. Allen, T. M., D. R. Mumbengegwi, and G. J. Charrois. Anti-CD19-targeted liposomal doxorubicin improves the therapeutic efficacy in murine B-cell lymphoma and ameliorates the toxicity of liposomes with varying drug release rates. Clin Cancer Res 11(9), 3567-73. (2005).
125. Wang, L., M. Fuster, P. Sriramarao, and J. D. Esko. Endothelial heparan sulfate deficiency impairs L-selectin- and chemokine-mediated neutrophil trafficking during inflammatory responses. Nat Immunol 6(9), 902-10. (2005).
126. Sugano, M., N. K. Egilmez, S. J. Yokota, F. A. Chen, J. Harding, S. K. Huang, and R. B. Bankert. Antibody targeting of doxorubicin-loaded liposomes suppresses the growth and metastatic spread of established human lung tumor xenografts in severe combined immunodeficient mice. Cancer Res 60(24), 6942-9. (2000).
127. Rajagopalan, K., G. Pavlinkova, S. Levy, P. R. Pokkuluri, M. Schiffer, B. E. Haley, and H. Kohler. Novel unconventional binding site in the variable region of immunoglobulins. Proc Natl Acad Sci USA 93(12), 6019-24. (1996).

### Example 3

### Selective Gene Silencing in Activated Leukocytes by Targeting siRNAs to the Integrin Lymphocyte Function-Associated Antigen-1

Expression of Anti-LFA-1 Fusion Proteins.

To incorporate an LFA1-targeting moiety into an siRNA delivery reagent, the heavy- and light-chain variable genes of the LFA-1 IgG antibodies TS1/22 (16) and AL-57 (13, 14) were converted to scFvs, which were fused at their C termini in a bacterial expression plasmid with the sequence for a basic peptide from human protamine, corresponding to amino acids 8-29, as described (7). AL-57-PF and TS1/22-PF were expressed in bacteria with a His6 tag and purified to homogeneity from the periplasm by sequential Ni-NTA affinity and ion-exchange chromatography (data not shown).

AL-57-PF Delivers siRNA to Silence Gene Expression Selectively in HA LFA-1-Expressing Cells.

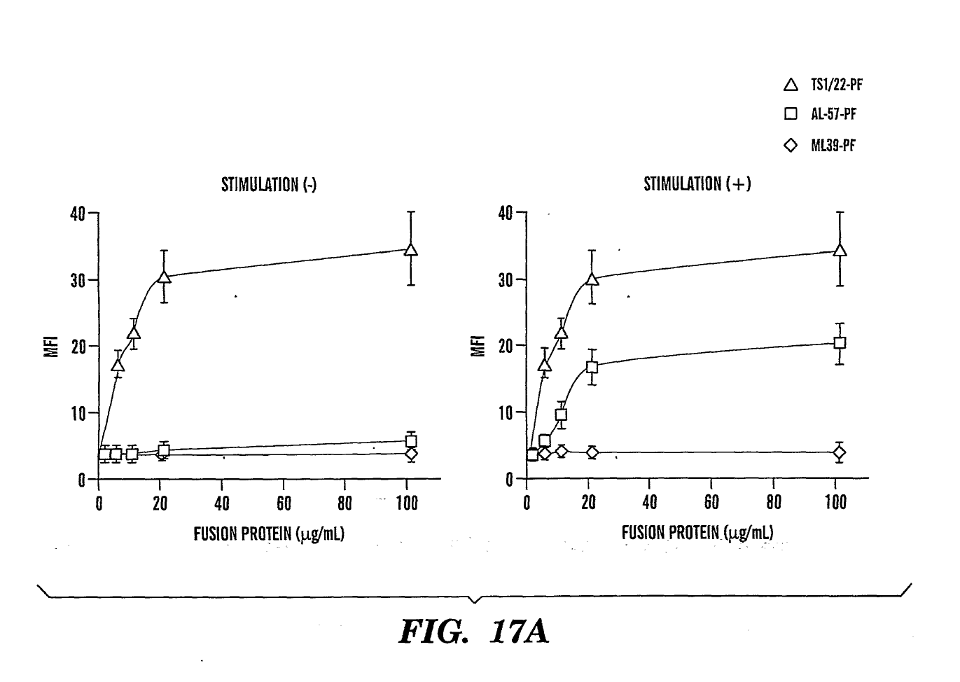

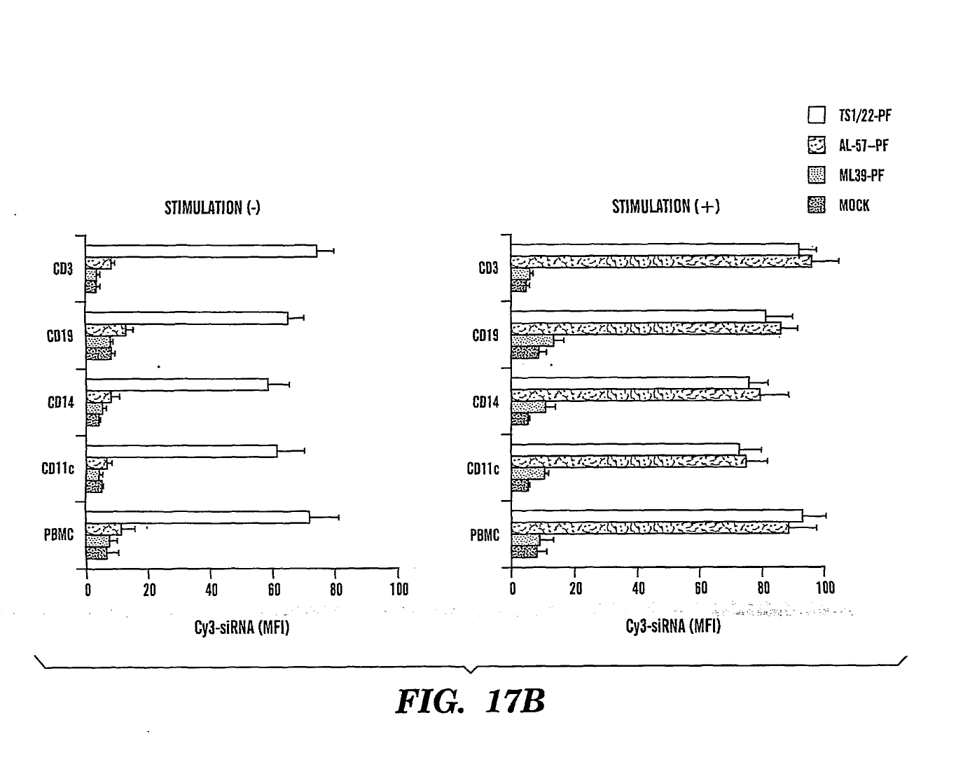

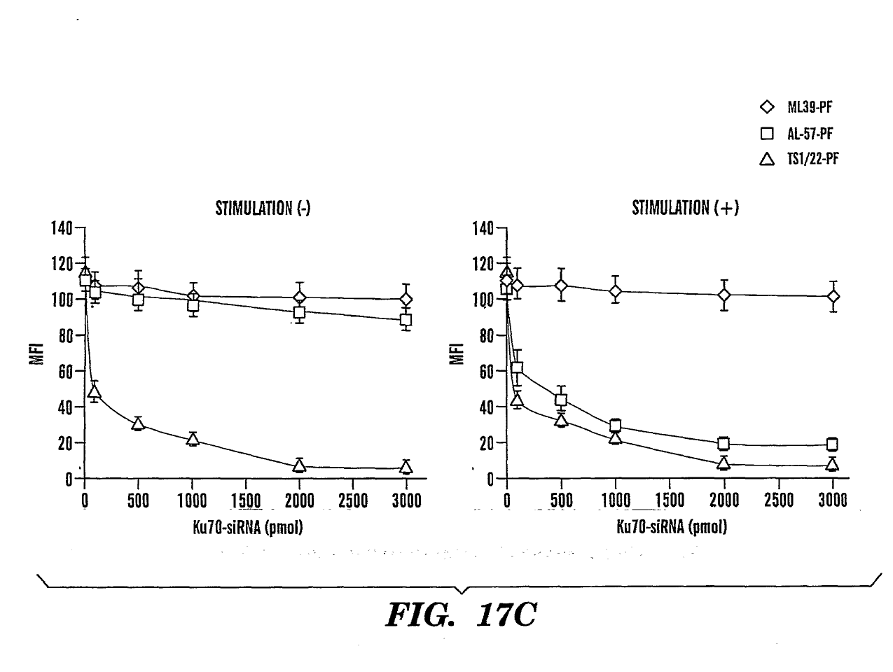

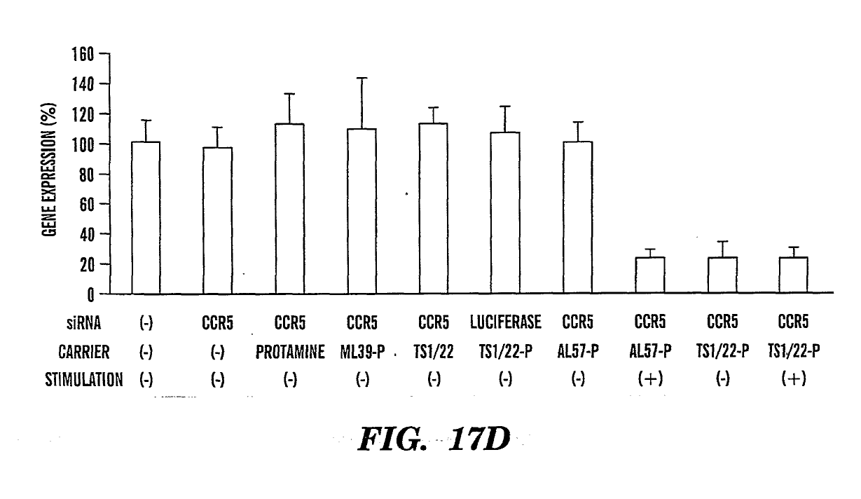

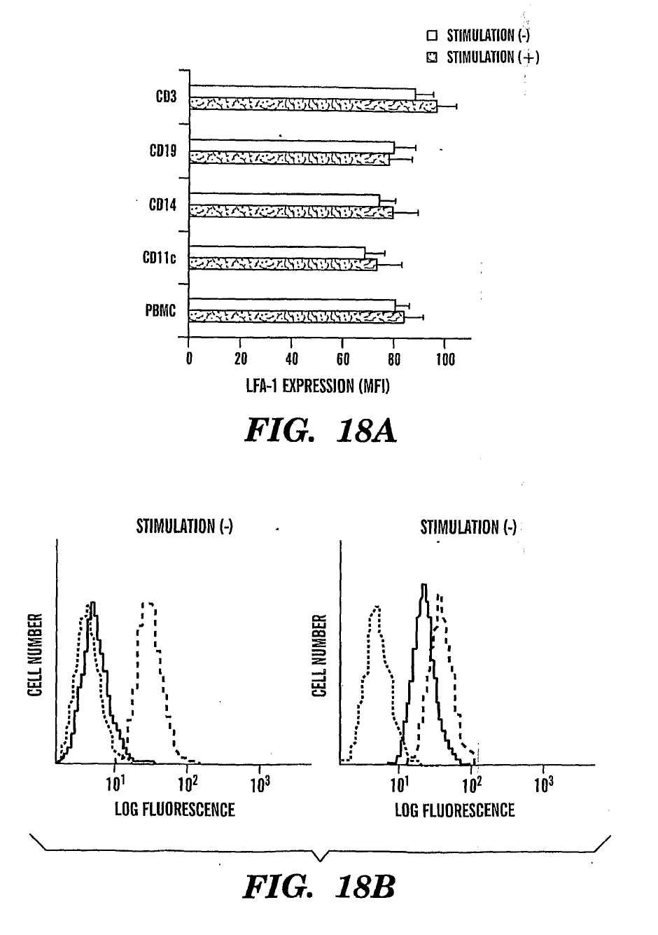

TS1/22 binds nonselectively to both low- and HA LFA-1 (17), whereas IgG AL-57 binds selectively to HA LFA-1 (13, 14). To verify that their binding specificities were preserved after conversion to scFv-PF, we used flow cytometry to assess binding to human peripheral blood mononuclear cells (PBMC) of Alexa-488-labeled AL-57-PF, TS1/22-PF, and ML39-PF [a control fusion protein that recognizes human ErbB2 (7)]. On circulating blood cells, LFA-1 is predominantly in the low-affinity form but can be converted to the HA form by stimulation in the presence of Mg^{2□} and EGTA with an activating antibody CBRLFA-1/2 (18). Stimulation with the activating antibody did not affect LFA-1 expression on any subset (FIG. 18A). TS1/22-PF bound to PBMC independently of stimulation, but AL-57-PF bound only to stimulated PBMC (FIG. 17A and FIG. 18B). Because the fusion proteins bound to cells with the specificity of their respective antibodies, we next tested whether AL-57-PF specifically delivered fluorescently labeled siRNAs only into stimulated PBMC. Cy3-siRNA on its own or complexed with ML39-PF did not get into any subset of PBMC. TS1/22-PF efficiently delivered Cy3siRNA to both unstimulated and stimulated PBMC of each subtype, CD3+ T and CD19+ B lymphocytes, CD14+ monocytes, and CD11c+ dendritic cells (FIG. 17B). In contrast, AL57-PF delivered Cy3-siRNA only to a small subset of unstimulated T and B lymphocytes (≈1-2%) in PBMC. These were present as a distinct Cy3+ peak on flow cytometry that was not present in the control samples treated with Cy3-siRNA mixed with medium, irrelevant antibody, or protamine (FIG. 17B and data not shown). These small subpopulations likely represent the small numbers of circulating activated lymphocytes in healthy donors. However, AL-57-PF potently delivered Cy3-siRNA to all subsets of stimulated PBMC (FIG. 17B). These results demonstrate the selective siRNA delivery by AL-57-PF only to activated leukocytes.

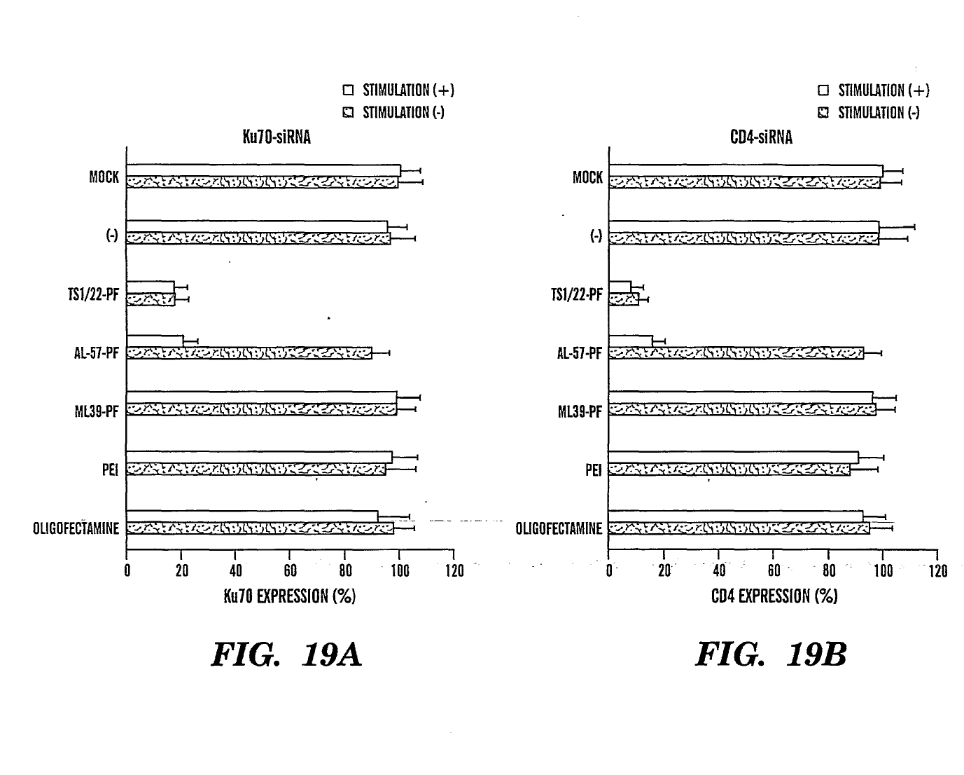

We next asked whether AL-57-PF-delivered siRNAs could induce silencing of the ubiquitously expressed Ku70 gene (7) selectively to HA LFA-1-expressing cells. Stimulated or unstimulated PBMC were analyzed 48 h after treatment with Ku70-siRNA delivered by polyethyleneimine (PEI), oligofectamine or scFv-PFs. siRNA complexed with PEI or oligofectamine did not significantly reduce Ku70 expression (FIG. 19), confirming that PBMC are resistant to conventional transfection reagents. Ku70-siRNA delivered by TS1/22-PF induced potent silencing independently of stimulation, whereas Ku70siRNA delivered by AL-57-PF induced silencing only in stimulated cells (FIG. 17C and FIG. 19A) Silencing was readily detectable with 100 pmol of siRNA and plateaued at ≈2,000 pmol (FIG. 17C). Similar results were obtained when CD4-siRNA was delivered to primary stimulated and unstimulated lymphocytes to silence CD4 expression (FIG. 19B).

AL-57-PF Silences Chemokine Receptor CCR5 Selectively in HA LFA-1 Expressing Cells.

CCR5 is a chemokine receptor that plays a critical role in Th1 type immunity to pathogens (19) and is a coreceptor for HIV infection (20). Aberrant up-regulation of CCR5 in T lymphocytes is implicated in the induction of Th1-type responses in rheumatoid arthritis and transplant rejection (21). Therefore, selective attenuation of CCR5 expression in activated lymphocytes might be a novel approach to treat autoimmune disease or HIV infection. To investigate the feasibility of this approach in vitro, we tested delivery of CCR5siRNA by LFA-1 antibody fusion proteins. Memory T cells express CCR5 and low-affinity LFA-1 that converts to the HA conformation after stimulation with CBRLFA-1/2 (14). Unstimulated or stimulated memory T cells were treated with CCR5-siRNA or control luciferase-siRNA mixed with the fusion proteins or their constituent components and analyzed by quantitative RT-PCR for CCR5 expression (FIG. 1D). Stimulation with CBRLFA-1/2 on its own did not alter CCR5 mRNA expression (not shown). As expected, CCR5-siRNA delivered by TS1/22-PF greatly reduced mRNA expression independently of stimulation, whereas CCR5 was reduced by CCR5-siRNA delivered by AL-57-PF only in stimulated lymphocytes. These results demonstrate potent and selective gene silencing only in activated PBMC and T lymphocytes after siRNA delivery with AL-57-PF and activation-independent gene silencing by siRNA delivered by TS1/22-PF in resting and activated mononuclear cells that are normally resistant to transfection.

AL-57-PF Selectively Targets HA LFA-1-Expressing Cells in Heterogeneous Populations.

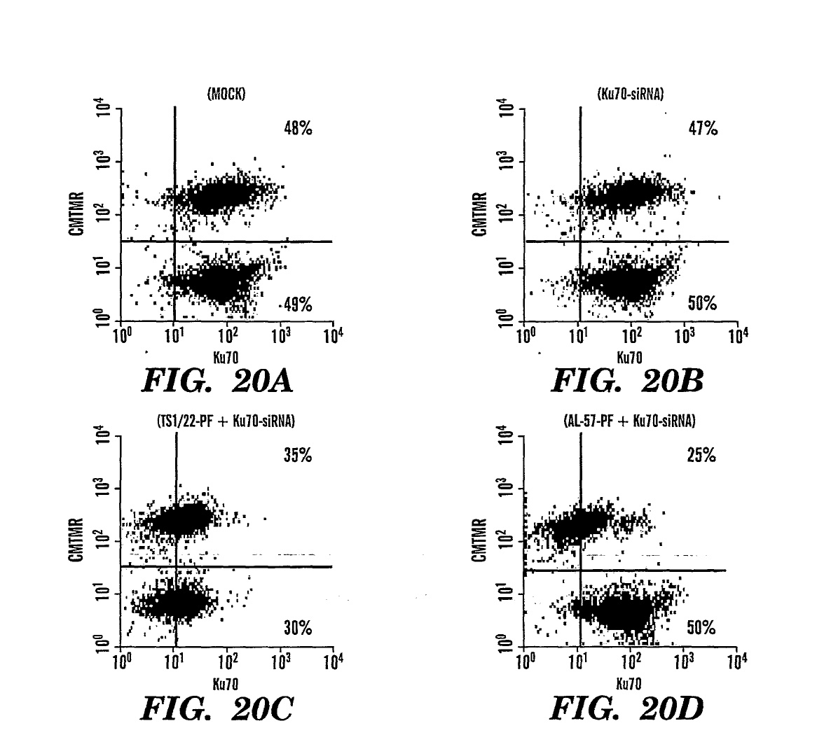

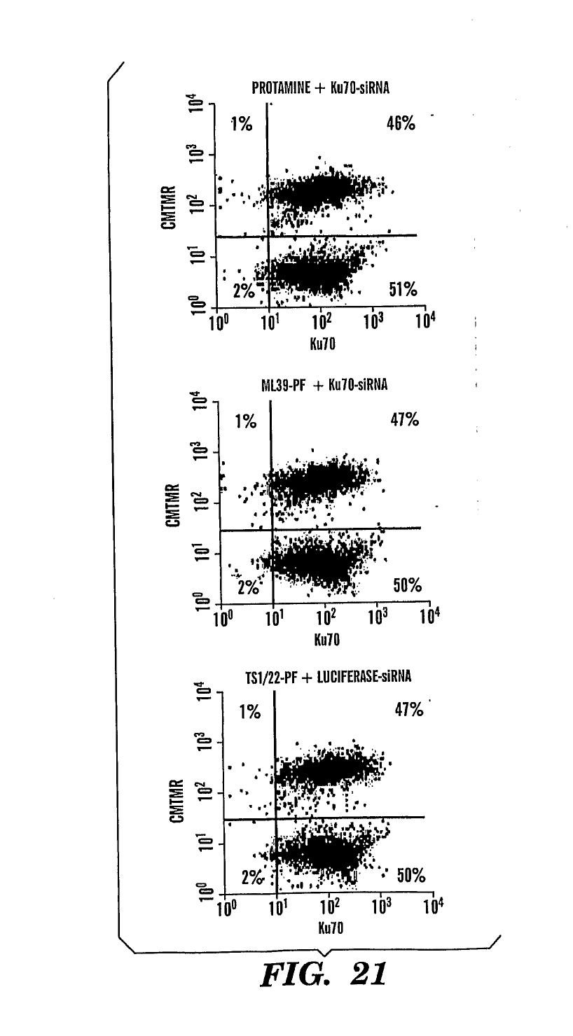

To demonstrate further the selective delivery of siRNAs to activated cells in heterogeneous populations of cells expressing both high- and low-affinity LFA-1, we delivered Ku70-siRNAs to mixed populations of K562 cells that were stably transfected to express LFA-1 (22) and then either exposed to the stimulating antibody CBRLFA-1/2 or the nonactivating control LFA-1 antibody TS2/4. The stimulated cells were labeled with CMTMR (CellTracker, Invitrogen, Carlsbad, Calif.) to identify them in the mixed population. Ku70-siRNA delivered by TS1/22-PF-reduced Ku70 protein expression in both CBRLFA1/2- and TS2/4-treated cells, showing gene silencing independent of LFA-1 activation (FIG. 20 and FIG. 21). In contrast, Ku70siRNA delivered by AL-57-PF selectively attenuated Ku70 expression in CBRLFA-1/2-treated cells, while leaving Ku70 expression in TS2/4-treated cells unchanged (FIG. 20). These results demonstrate siRNA delivery by AL-57-PF selectively targets HA LFA-1 expressing cells in heterogeneous populations.

AL-57-PF Delivers siRNA to Silence Gene Expression in Lymphocytes Activated by T Cell Receptor (TCR) or Chemokine Stimulation.

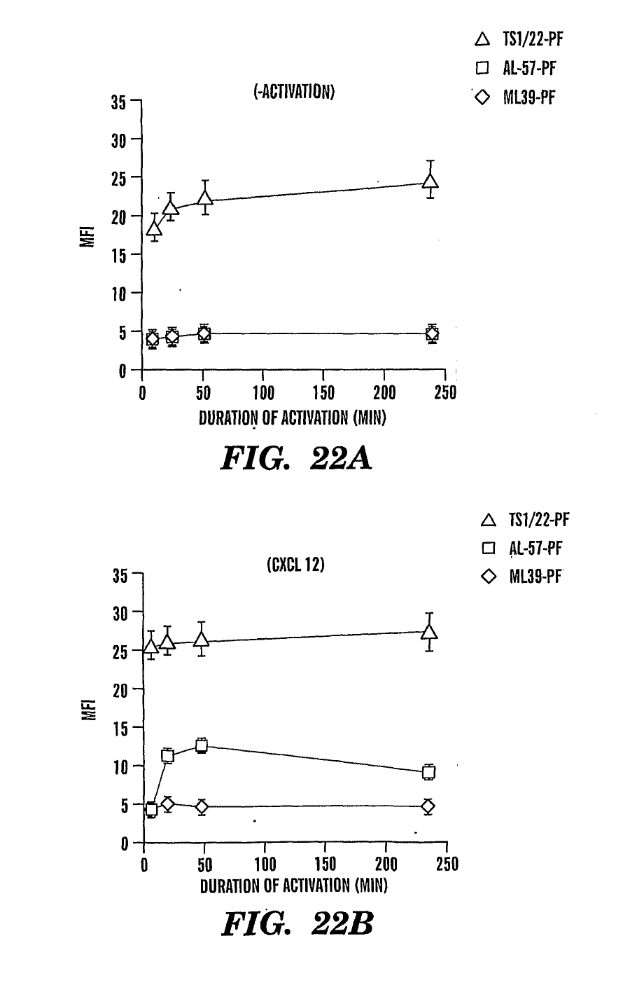

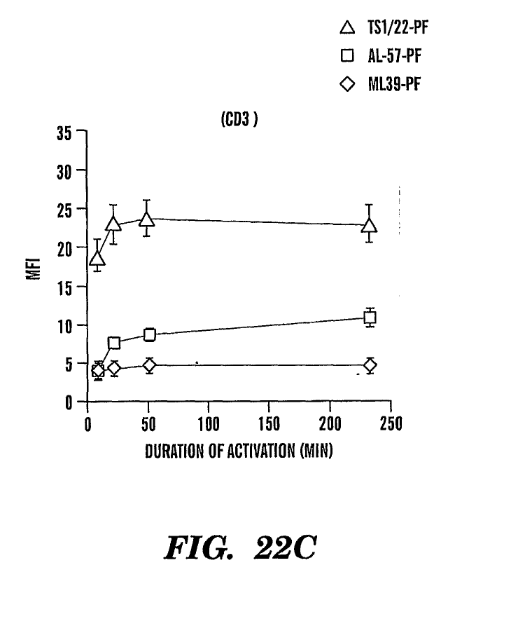

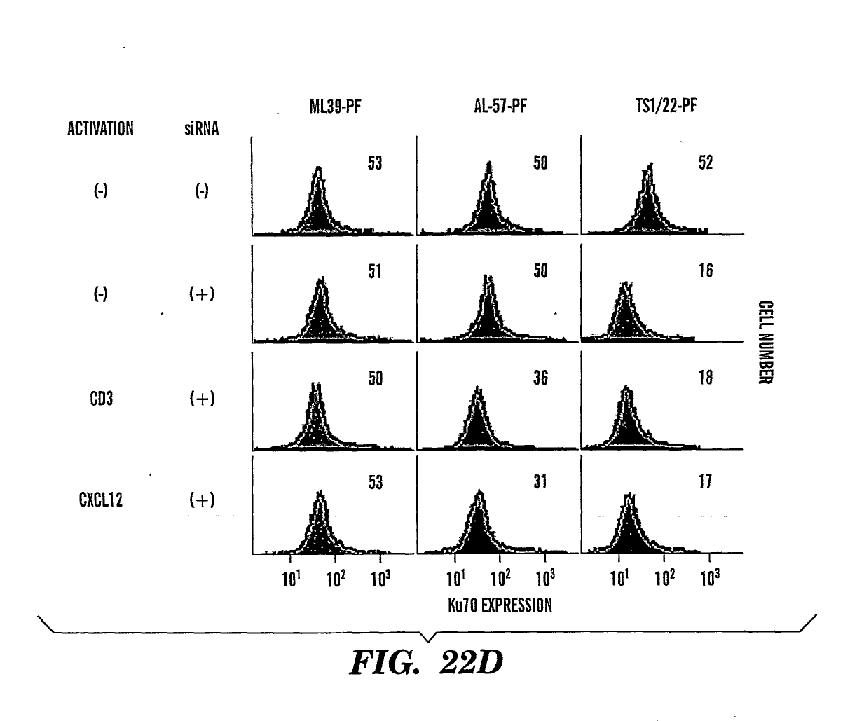

Activation of lymphocytes by engagement of the TCR or chemokine receptors elicits intracellular signaling cascades that lead to transient up-regulation of HA LFA-1 (12). During chronic inflammation, the HA conformation of LFA-1 persists in aberrantly activated lymphocytes (23, 24). To investigate whether AL-57-PF delivers siRNAs and silences gene expression in lymphocytes activated by physiologically relevant stimuli that model chronic inflammation in vitro, T lymphocytes were exposed to immobilized CD3 antibody or immobilized CXCL12 chemokine, which elicit persistent activation of LFA-1. Binding of TS1/22-PF and AL-57-PF was used to verify the effects of these stimuli on LFA-1 conformation. As determined by binding of activation-insensitive TS1/22-PF, LFA-1 expression barely changed during activation (FIG. 22A-C). Unstimulated normal donor T lymphocytes did not bind AL-57-PF in the absence of stimulation (FIG. 22A), but binding persisted for at least 4 h after exposure to the immobilized stimuli (FIGS. 22B and C).

We next used confocal microscopy to investigate the ability of Alexa-488-labeled AL-57-PF to bind and deliver Cy3-siRNA selectively to activated lymphocytes. Four hours after exposure of activated lymphocytes to the fluorescently labeled fusion protein-siRNA complexes, Alexa488-AL-57-PF was distributed to both the plasma membrane and internal punctuate structures, whereas Cy3-siRNA was predominantly intracellular, colocalizing with the fusion protein. The conformation-sensitive fusion protein AL-57-PF did not transduce unactivated lymphocytes. As expected, T cells treated with Alexa-488-TS1/22-PF internalized Cy3-siRNA with a similar staining pattern, but uptake was independent of cell activation. These observations indicate that Cy2-siRNA and Alexa-488 TS1/22-PF were taken up by unstimulated T cells, whereas uptake of Alexa-488-AL-57-PF required T cell activation. Using Ku70-siRNA, we next investigated the ability of AL 57-PF to silence genes selectively in lymphocytes activated by physiologic stimuli. Activation by immobilized CD3 mAb or CXCL12 did not affect Ku70 protein expression assessed by flow cytometry. Exposure to AL-57-PF-siRNA complexes reduced Ku70 selectively in activated lymphocytes, whereas TS1/22-PF-siRNAs reduced Ku70 levels even in unstimulated lymphocytes (FIG. 22E and data not shown). These results demonstrate that AL-57-PF enables the manipulation of gene expression selectively in lymphocytes activated by physiologically relevant stimuli.

AL-57-PF-Mediated Knockdown of Cyclin D1 Suppresses Proliferation Selectively in Activated Lymphocytes.

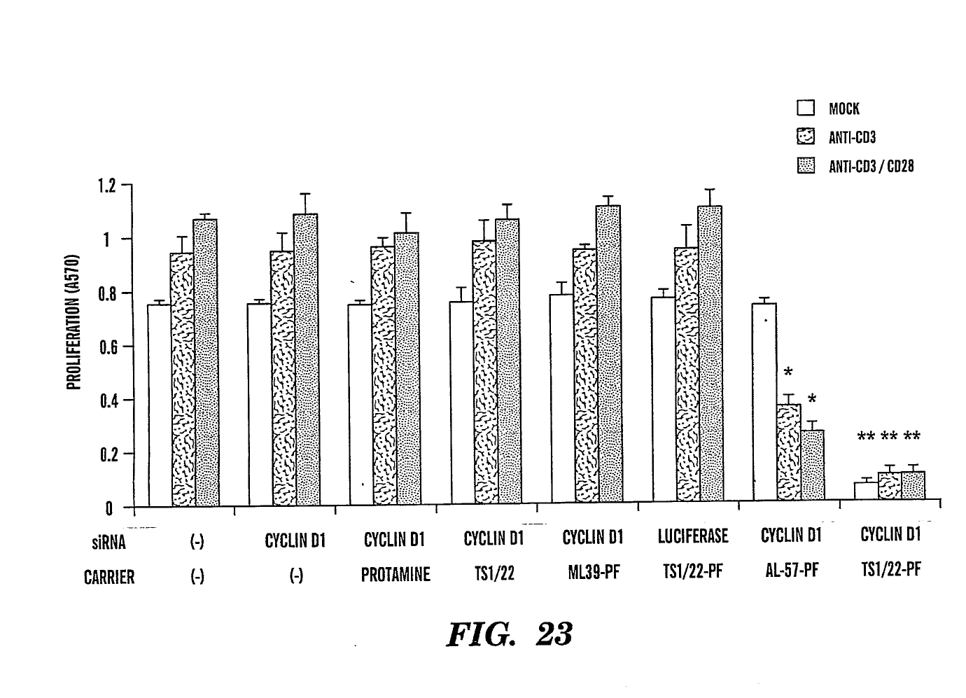

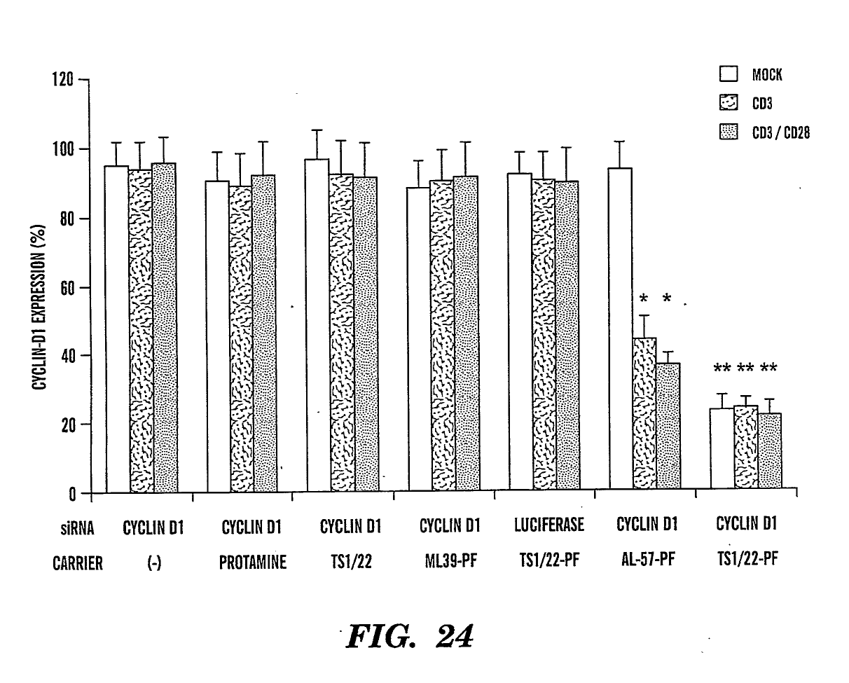

Proliferation of aberrantly activated lymphocytes has been implicated in the pathogenesis of autoimmune diseases. Cyclins and other cell-cycle regulating proteins represent a potential therapeutic target for autoimmune diseases and other diseases caused by overly exuberant immune activation (25). We therefore investigated whether we could selectively suppress cellular proliferation in activated lymphocytes using cyclin D1-siRNA. Basal proliferation of memory T cells, prepared by in vitro exposure of PBMC to IL-15, was enhanced by activation with immobilized CD3-mAb alone or together with CD28-mAb (FIG. 23). Proliferation measured by the 3-(4,5-dimethylthiazol-2-yl)-2,5-diphenyl tetrazolium bromide (MTT) assay was not altered by exposure to cyclin D1-siRNA alone or mixed with protamine, TS1/22-scFv, ML39-PF. Luciferase-siRNA delivered by TS1/22-PF also had no effect on lymphocyte proliferation. However, cyclin D1-siRNA delivered by TS1/22-PF potently inhibited basal proliferation of memory T cells as well as the elevated proliferation of activated lymphocytes. Cyclin D1-siRNA delivered by AL-57-PF did not affect proliferation of unactivated memory T cells but significantly suppressed proliferation in activated lymphocytes (FIG. 23). Moreover, suppression was somewhat more effective in cells that were more fully stimulated by both antibodies. Experiments with CD3 and CD28 mAbs immobilized at 1 and 5 μg/ml produced similar results (FIG. 23 and data not shown). Proliferation measured by [³H] thymidine incorporation showed similar results (not shown). Suppression of proliferation correlated with levels of cyclin D1 knockdown (FIG. 24).

The Fusion Proteins Targeting LFA-1 Deliver siRNA In Vivo.

We next investigated whether LFA-1-targeted fusion proteins could deliver siRNA in vivo. Because AL-57 and TS1/22antibodies do not recognize murine LFA-1, we used SCID mice engrafted with K562 cells stably transfected to express human WT LFA-1 (K562-WT LFA-1) or HA LFA-1 (K562-HA LFA-1) (22, 26). Five days after i.v. injection of K562 cells, when they formed numerous small nodules in the lung (not shown), we injected 1.2 nmol (40 μg) of fusion protein complexed with 6 nmol (100 μg) of Cy3-siRNA into the tail vein.

Four hours later, lung tissues were harvested and examined by immunohistochemistry, and single-cell suspensions were analyzed by flow cytometry for uptake of fluorescent siRNA. Four hours after injection of Cy3-siRNA complexed with AL-57 or TS1/22-PF, siRNA delivery to K562 cells in the lungs of SCID mice, examination by fluorescence microscopy indicated that TS1/22-PF delivered siRNA equally well to cells expressing WT and HA-LFA-1. By contrast, AL-57-PF preferentially delivered to K562-HA LFA-1. Mouse lung cells did not take up the siRNA. Cy3-siRNA complexed with a control fusion protein (ML39-PF) was not taken up above background (Table 2 and data not shown). TS1/22-PF delivered Cy3-siRNA equally well to K562-WT LFA-1 and K562-HA LFA-1 but not to mouse lung cells. AL-57-PF delivered Cy3siRNA to K562-HA LFA-1 as well as TS1/22-PF, but siRNA delivery to K562-WT LFA-1 was much less efficient than delivery by TS1/22-PF. Neither protein induced significant uptake of Cy3-siRNA by parent K562 cells that do not express LFA-1 (not shown). The collective data indicates specific in vivo siRNA delivery by anti-LFA-1 fusion proteins to K562 cells expressing human WT LFA-1 or human HA LFA-1. These results demonstrate in vivo proof of principle for the effective systemic siRNA delivery by TS1/22-PF to LFA-1-expressing cells and the selective delivery by AL-57-PF to HA LFA-1-expressing cells.

TABLE 2
In vivo siRNA delivery to K562 cells expressing
LFA-1 in SCID mice

| | Cy3⁺ K562 cells/Total K562 cells, % | | | |
|---|---|---|---|---|
| | Fusion | TS1/22- | AL-57- | ML39- |
| Cells | protein | PF | PF | PF |
| K562-WT | | 88.5 ± 7.0 | 23.3 ± 3.2 | 4.7 ± 2.2 |
| LFA-1* | | (9) | (9) | (9) |
| K562-HA | | 87.4 ± 7.4 | 88.1 ± 6.8 | 4.2 ± 1.5 |
| LFA-1** | | (6) | (6) | (6) |

In vivo Cy3-siRNA delivery to K562 cells engrafted in the lungs of SCID mice was quantified by flow cytometry analysis of single-cell suspensions of lung tissues. K562 cells were identified by staining with FITC-human CD45 mAb. The number of mice used is shown in parentheses. Data are mean ± SD of three (*) and two (**) independent experiments.

Exposure to siRNA-Fusion Protein Complex Does Not Cause Lymphocyte Activation.

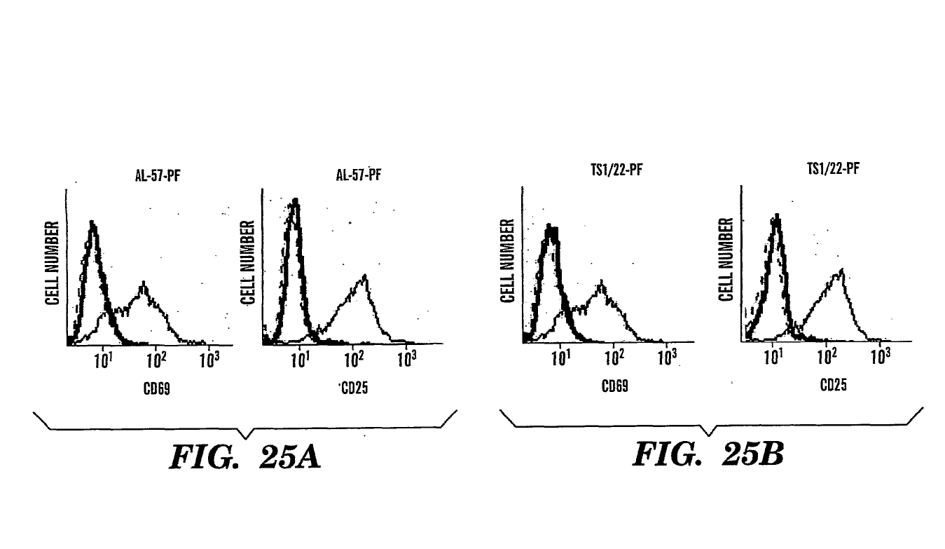

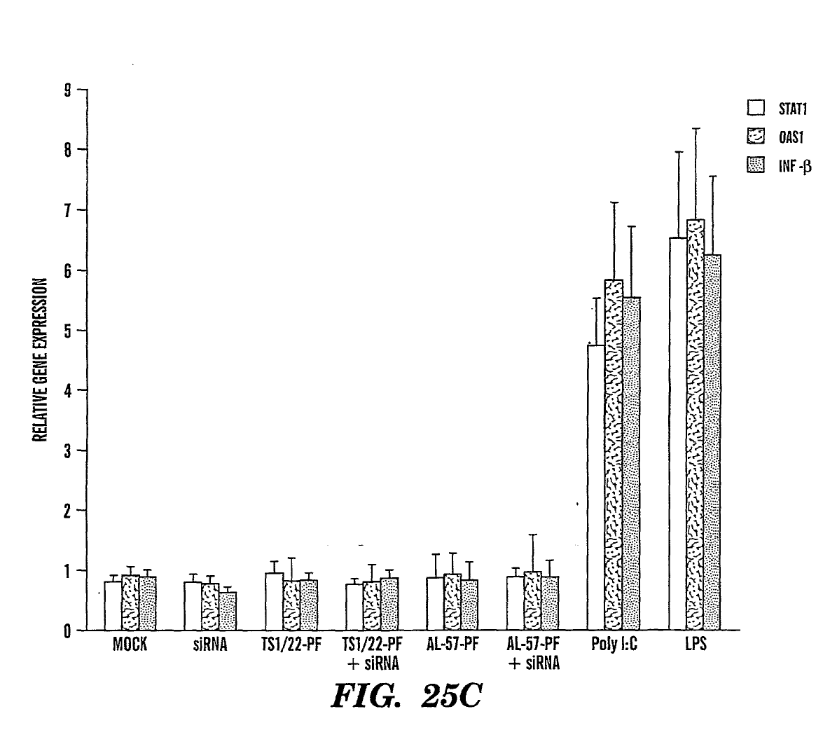

The engagement of LFA-1 by its natural ligand intercellular adhesion molecule-1 leads to lymphocyte activation (27). The LFA-1 targeting fusion proteins might have limited usefulness if they activated the cells they targeted. To determine whether TS1/22-PF or AL-57-PF complexes cause lymphocyte activation, we measured the induction of the early activation markers CD25 and CD69 on PBMC cultured for 48 h with the fusion proteins mixed with luciferase-siRNA. Neither fusion protein-siRNA complex induced expression of CD69 and CD25, whereas activation with phytohaemagglutinin induced expression of both markers (FIGS. 25A and B). Therefore, transducing lymphocytes with the anti-LFA-1 scFv fusion proteins does not activate them.

LFA-1-Targeted siRNAs Do Not Elicit IFN Responses.

Another possible unwanted off-target effect of fusion protein-delivered siRNA would be activation of IFN-responsive genes (IRG) by activating cytosolic dsRNA-activated protein kinase PKR or by binding to Toll-like receptors 3, 7, and 8 that recognize RNA on the cell surface or in endosomes (28, 29). To examine whether AL-57 PF- or TS1/22-PF-siRNA complexes activate an IFN response, we used quantitative RT-PCR to measure mRNA expression of IFN-β, and two key IRG, 2′,5′-oligoadenylate synthetase and Stat-1 (7) in PBMC stimulated with Mg/EGTA plus CBRLFA1/2 and then treated with the fusion protein-siRNA complexes (FIG. 25C). The IRG were not induced by the LFA-1 antibody but were induced by treatment with the known IFN inducers, LPS and poly(I:C). Treatment with as much as 1 μM luciferasesiRNA delivered by TS1/22-PF or AL-57-PF did not induce an IFN response. Therefore, even in highly sensitive primary cells, siRNA complexed with the scFv-protamine fragment fusion proteins does not trigger nonspecific IFN responses.

Discussion

Here we report LFA-1-targeted scFv-protamine fusion proteins as a nonviral delivery approach to induce RNAi in primary leukocytes. Primary lymphocytes are highly resistant to nonviral siRNA delivery with cationic lipid and polymer reagents (3-5), as confirmed in the present study. By fusing LFA-1-specific scFv antibodies (AL-57 and TS1/22) to a protamine fragment that condenses siRNA through charge interactions, we have developed an LFA-1-specific delivery method for efficient gene silencing. The conformation-insensitive TS1/22-PF enables potent gene silencing in all leukocytes independently of activation status. These include primary lymphocytes and dendritic cells, which are resistant to conventional transfection techniques. Furthermore, the HA LFA-1-specific AL-57-PF enables gene silencing selectively in activated leukocytes. Moreover, IFN responses were not triggered in primary cell PBMC mixtures containing highly sensitive antigen-presenting cells. Finally, using SCID mice engrafted with K562 cells expressing WT or HA LFA-1, we demonstrated the in vivo feasibility of activation-independent delivery by TS1/22-PF to LFA-1-bearing cells and activation-dependent delivery by AL-57-PF.

These results support the general applicability and high degree of specificity possible with antibody-protamine fusion proteins for targeted siRNA delivery to primary cells. Moreover, the targeting fusion proteins do not activate lymphocytes, even though they engage a cell surface signaling molecule. This may be because the targeting reagent is monomeric, because it is designed from a scFv and is not expected to cross-link the receptor.

AL-57 is a ligand mimetic antibody that binds selectively to the HA conformation of LFA-1 (14). LFA-1 activation by a single encounter with an activating stimulus is transient; stimulation of lymphocytes with soluble anti-CD3 antibody (30) and soluble chemokine CXCL12 (31) increases LFA-1 adhesiveness only for 5-20 min. In contrast, as we found here using immobilized stimuli that constitutively engage TCR or CXCR4, sustained receptor engagement leads to persistent affinity up-regulation of LFA-1. Constitutive lymphocyte activation might mimic aberrant activation in chronic inflammation. To examine the potential therapeutic feasibility of AL-57-PF-directed siRNA delivery, we investigated whether we could suppress lymphocyte proliferation selectively in persistently stimulated populations. Cyclin D1-siRNA delivered by AL-57-PF suppressed lymphocyte proliferation only when cells were stimulated with CD3 or CD3/CD28. By contrast, TS1/22-PF suppressed lymphocyte proliferation independently of the state of lymphocyte activation. There are several potential therapeutic advantages of selective gene silencing. Selective targeting of activated lymphocytes would likely be sufficient to suppress inflammatory tissue injury. By leaving resting and naïve cells untouched, selective targeting would reduce iatrogenic immunodeficiency, a major problem associated with current immunosuppressive drugs (15). Moreover, the siRNA dose required to target a small subset of disease-causing cells is likely to be substantially less than that needed for indiscriminate targeting.

Other activation markers, such as CD69, CD25, CD40L, or OX40, could also be used for selective targeting of activated lymphocytes (15, 32, 33). The expression profiles of cell surface molecules after activation vary greatly depending on timing and the character and strength of the activating stimulus (32). Fusion proteins, based on antibodies or ligands to different activation markers, might allow targeting of overlapping but distinct phases of lymphocyte activation. Determining which targeting strategy would be most appropriate for different pathological conditions will require in vivo studies.

This study inducates that LFA-1-directed siRNA delivery reagents are useful for targeting leukocytes in vivo for research to understand disease pathogenesis or discover useful drug targets or for RNAi-based therapy. LFA-1 is expressed on the surface of all leukocytes. Although methods have recently been described for efficient systemic siRNA delivery to the liver (34-36), so far there are no clinically relevant in vivo examples of systemic siRNA delivery to other organs or to moving targets, such as hematopoietic cells. Moreover, the ability to transduce only activated subsets of immune cells by taking advantage of the conformational change of LFA-1 on activated cells provides the potential for highly targeted research or therapeutic intervention. Although this study was done with human reagents that do not recognize mouse LFA-1, and we are currently engineering the murine analogs for in vivo testing, the feasibility study using SCID mice engrafted with K562 cells expressing human LFA-1 strongly supports the applicability of LFA-1 antibody fusion proteins for in vivo siRNA delivery. In SCID mice, whereas the selectivity of AL-57-PF to deliver siRNA to HA LFA-1 over WT LFA-1 was well maintained, K562-WT LFA-1, which rarely takes up siRNA in vitro (FIG. 20D and data not shown), showed some uptake of siRNA delivered by AL-57-PF (Table 2). This result suggests that WT LFA-1 in K562 transfectants may be activated in vivo by binding to intercellular adhesion molecule-1 in homotypic cell aggregates (37) and/or by the innate inflammatory responses elicited by xenogeneic reactions to K562 cells.

Targeting LFA-1 using siRNA-fusion protein complexes might have enhanced efficacy at suppressing immune activation and inflammation compared with other ways of delivering siRNA. Many LFA-1 antibodies, including AL-57, block leukocyte adhesion (13, 38), and LFA-1 blocking mAbs are effective in attenuating inflammatory disease in mouse models and in treating psoriasis patients (39, 40). Targeted siRNA delivery using blocking LFA-1 antibodies might produce additive or synergistic effects by both silencing proinflammatory molecules and inhibiting LFA-1-mediated cell adhesion. Because blocking LFA-1 by itself is insufficient to suppress inflammation in certain disease models (41), combining LFA-1-blocking antibodies with gene silencing might be a more powerful therapeutic approach.

### Example 3

### Methods

siRNA Delivery and Gene Silencing.

siRNAs mixed with fusion proteins (in a 5:1 molar ratio), appropriate controls (i.e., scFv, protamine), or vehicles in 50 μl of PBS were preincubated for 30 min at room temperature and added to 2×10⁵ PBMC or lymphocytes in 150 μl of RPMI medium 1640/10% FCS in the presence of 1 mM MgCl2/CaCl2 or 5 mM MgCl2/1 mM EGTA plus 10 μg/ml mAb CBRLFA-1/2. Cells were cultured for 6-72 h at 37° C., 5% CO2 and subjected to flow cytometry and/or RT-PCR analyses.

T Lymphocyte Activation Through CXCR4 and TCR.

Microtiter plates were coated for 1 h at 37° C. with CXCL12 (5 μg/ml), anti-human CD3 mAb (5 μg/ml; clone 1304; Immunotech, Marseille, France), and/or anti-human CD28 mAb (5 μg/ml; clone 1373; Immunotech), washed, and blocked with RPMI medium 1640 containing 10% FCS for 1 h at 37° C. T lymphocytes (1×10⁵ cells per well in 100 μl) were stimulated for the indicated times at 37° C., 5% CO2. To study the kinetics of fusion protein binding, Alexa-488-labeled fusion proteins (20 μg/ml) were added 15 min before the end of stimulation. Cells were fixed in cold 2% formaldehyde in Hanks' balanced salt solution (HBSS), washed three times with HBSS containing 2% glucose and 2% BSA, resuspended in HBSS, and analyzed by flow cytometry. To study siRNA delivery, cells were treated for 4 h with Cy3-siRNA on its own or delivered by fusion proteins and analyzed with fluorescent microscopy. To study silencing, cells were cultured for 3 days in the presence or absence of Ku70-siRNA alone or complexed with fusion proteins and analyzed with flow cytometry.

Mixed-Population Transduction Experiment.

K562 cells transfected to express LFA-1 were either treated for 30 min at 37° C. with the activating antibody CBRLFA-1/2 (10 μg/ml) and labeled with 4 μM CMTMR (CellTracker, Invitrogen) or treated with the nonactivating LFA-1 antibody TS2/4 and mock-labeled. The two populations were washed and mixed in equal numbers and then cocultured for 48 h at 37° C., 5% CO2, in RPMI medium 1640/10% FCS in the presence of 1 nmol of Ku70-siRNA or luciferase-siRNA, alone or complexed with protamine or an indicated antibody-protamine fusion protein, before measuring intracellular Ku70 expression by flow cytometry.

In Vivo Delivery.

SCID mice on a CB17 background (5-7 weeks old) from Charles River Breeding Laboratory (Wilmington, Mass.) were injected by tail vein with 6×10⁶ K562 cells transfected to express WT or HA LFA-1. Five days later, Cy3-siRNA (6 nmol) complexed with fusion protein in a 5:1 molar ratio in 100 μl of PBS was injected by tail vein. Mice were sacrificed 4 h later, and the lungs were harvested. The right lung was placed in optimal cutting temperature compound (Tissue-Tek, Hatfield, Pa.) snap-frozen in liquid nitrogen, and used for immunohistochemistry. Single-cell suspensions, prepared by mechanical disruption of the left lung, were analyzed by flow cytometry. All animal procedures were approved by the Animal Care and Use Committee of the CBR Institute for Biomedical Research.

Construction and Expression of scFv and scFv-Protamine Fusion Proteins.

mRNA isolated from the TS1/22 hybridoma (provided by Timothy A. Springer, CBR Institute for Biomedical Research and Harvard Medical School) (Haskard et al., (1986) J Immunol 137:2901-2906) was converted into cDNA. A plasmid containing AL-57 cDNA was previously described (Huang et al. (2006) J Leukocyte Biol 80:905-914). Using overlapping PCR, the heavy and light chain variable domains of TS1/22 and AL-57 were amplified and engineered into an scFv plasmid with a (G₄S)₄ linker (Jin et al., (2006) Proc Natl Acad USA 103:5758-5763). The scFv cDNAs were subcloned into pET-26b (Novagen) that encodes for a C-terminal His-6 tail. To generate the scFv-protamine fragment fusion protein (scFv-PF) cDNA, overlapping PCR was used to fuse the scFv cDNA in frame to the N terminus of the cDNA fragment encoding Arg-8 to Ser-29 of human protamine, which was then subcloned into pET-26b. All constructs were verified by DNA sequencing. scFv and scFv-PF proteins were expressed in BL21(DE3) (Novagen), and purified from the periplasm by Ni-NTA affinity chromatography followed by ion-exchange chromatography with mono Q HR5/5 (Pharmacia) for scFv and mono S HR5/5 (Pbarmacia) for scFv-PF. The pooled fractions were dialyzed against PBS and then PBS with 5% glycerol and stored at −80° C. The control scFv-PF fusion protein that recognizes human ErbB2 (ML39-PF) was previously described (Li et al., (2001) Cancer Gene Ther 8:555-565; Song et al. (2005) Nat Biotechnol 23:709-717).

PBMC and Memory T Lymphocytes.

CD4 T cells were isolated from normal donor PBMC by selection with human CD4 immunomagnetic beads (Miltenyi Biotec). Memory T cells were prepared by culturing PBMC in RPMI 1640 medium containing 10% FCS for 3 d in the presence of 4 μg/ml phytohemagglutinin (PHA), followed by treatment with IL-15 (10 ng/ml) for 3 d.

Preparation of siRNAs.

siRNAs from Dharmacon were deprotected and annealed according to the manufacturer's instructions. Four Ku70-siRNAs were used in an equimolar ratio as previously reported (Zhu et al., (2006) EMBO Rep 7:431-437). CCR5— and CD4-siRNAs were previously reported (Song et al. (2003) J Virol 77:7174-7181; Lee et al. (2005) Blood 106:818-826). Cyclin-D1-siRNAs (sc-29286) were from Santa Cruz Biotechnology (Santa Cruz, Calif.). The sense and anti-sense of siRNAs were: Cy3-luciferase, 5′-Cy3-CGUACGCGGAAUACUUCGAdTdT-3′(sense) (SEQ ID NO: 8), 5′-UCGAAGUAUUCCGCGUACGdTdT-3′ (antisense) (SEQ ID NO: 9); luciferase, 5′-CGUACGCGGAAUACUUCGAdTdT-3′ (sense) (SEQ ID NO: 10), 5′-UCGAAGUAUUCCGCGUACGdTdT-3′ (antisense) (SEQ ID NO: 11).

siRNA Transfection.

siRNA transfection with PEI (ExGene 500, Fermentas Life Science), Oligofectamine (Invitrogen), and Fugene 6 (Roche) was preformed according to manufacturer's instructions.

Quantitative RT-PCR.

Total RNA (1 μg) isolated with TRIzol (Invitrogen Life Technologies) was reverse-transcribed by using Superscript III (Invitrogen) and random hexamers, according to the manufacturer's protocol. Real-time quantitative PCR was performed on 1 μl of cDNA or a comparable amount of RNA with no reverse transcriptase, using Platinum Taq Polymerase (Invitrogen) and a Bio-Rad iCycler. SYBR green (Molecular Probes) was used to detect PCR products. All reactions were done in a 25-μl reaction volume in triplicate. The following primers were used:

(SEQ ID NO: 12)
CCR5(forward),
5′-TGTTTGCGTCTCTCCCAGGAATCA-3′
(SEQ ID NO: 13)
CCR5(reverse),
5′-AGCCCTGTGCCTCTTCTTCTCATT-3′
(SEQ ID NO: 14)
β-actin(forward),
5′-TGACGGCTGTCACCCACACTGTGGCCATCTA-3′
(SEQ ID NO: 15)
β-actin(reverse),
5′-CTAGAAGCATTTGCGGTGGACGATGGAGGG-3′
(SEQ ID NO: 16)
STAT1(forward),
5′-CGGTTGAACCCTACACGAAG-3′
(SEQ ID NO: 17)
STAT1(reverse),
5′-ACThrCCAAAGGCATGGTC-3′
(SEQ ID NO: 18)
OAS1(forward),
5′-GCAGAAAGAGGGCGAGTTC-3′
(SEQ ID NO: 19)
OAS1(reverse),
5′-TACTGAGGTGGCAGCTTCC-3′
(SEQ ID NO: 20)
IFNβ(forward),
5′-CCTGTTGTGCTTCTCCAC-3′
(SEQ ID NO: 21)
IFNβ(reverse),
5′-ATGTCAAAGTTCATCCTGTC-3′

PCR parameters consisted of 5 min of Taq activation at 95° C., followed by 40 cycles of PCR at 95° C.×20 sec, 60° C.×30 sec, and 69° C.×20 sec. Standard curves were generated and the relative amount of target gene mRNA was normalized to β-actin mRNA. Specificity was verified by melt curve analysis and agarose gel electrophoresis.

Lymphocyte Proliferation.

Lymphocyte proliferation was assayed by 3-(4,5-dimethylthiazol-2-yl)-2,5-diphenyl-2H-tetrazolium bromide (MTT) assay as described (Cerroni et al., (2002) Biomol Eng 19:119-124).

Flow Cytometry.

Flow cytometry of cell surface antigens was performed as described (5). The following mAbs were used: FITC- or PE-conjugated mAbs to CD3, CD4, CD19, CD14, CD11c (BD Bioscience); APC-conjugated mAbs to CCR5 (BD Bioscience); FITC- or PE-conjugated mAbs to CD45 (Immunotech); FITC-conjugated anti-6His tag (Zymed); mAb to protamine (Santa Cruz Biotechnology); FITC and Cy3-conjugated anti-goat and anti-human Ig secondary antibodies (Zymed). mAbs to integrin αL (TS2/4, TS1/22) and β2 (TS1/18) subunits were gifts from Timothy A. Springer and were labeled with Alexa 488 using an Alexa dye kit (Invitrogen).

For measuring intracellular expression of cyclin D1, cells were fixed and permeabilized with the Fix-and-Perm kit (Caltag Laboratories, Burlingame, Calif.), stained with 1 μg/ml goat anti-human cyclin D1 (Santa Cruz Biotechnology) on ice for 30 min, and counterstained with FITC-conjugated rabbit anti-goat IgG (Zymed). Detection of Ku70 expression was as described (6).

Data were acquired and analyzed on FACScan or FACS calibur with CellQuest software (Becton Dickinson, Franklin Lakes, N.J.).

Image Acquisition and Processing.

Confocal imaging was performed using a Bio-Rad Radiance 2000 Laser-scanning confocal system (Hercules, Calif.) with an Olympus BX50BWI microscope using an Olympus 100×LUMPlanFL 1.0 water-dipping objective. Image acquisition was performed using Laserscan 2000 software and image processing was performed with Openlab 3.1.5 software (Improvision, Lexington, Mass.).

Immunohistochemistry.

Frozen sections (5 μm thick) were air-dried, fixed in precooled acetone for 10 min, washed three times with PBS, and blocked with 10% FCS/PBS for 10 min. Sections were then incubated with FITC-labeled anti-human CD45 mAb (Immunotech) in 10% FCS/PBS overnight at 4° C. in the dark. After washing three times with PBS, sections were incubated with 400 nM DAPI in PBS at room temperature for 10 min. Slides were washed three times with PBS, air-dried for 10 min, and mounted with VECTASHIELD mounting medium (Vector Laboratories, Burlingame, Calif.). Mounted slides were observed with an Axiovert 200M inverted microscope (Zeiss).

### REFERENCE LIST FOR EXAMPLE 3

1 Behlke M A (2006) Mol Ther 13:644-670.
2 Dykxhoom D M, Lieberman J (2005) Annu Rev Med 56:401-423.
3 Goffinet C, Keppler O T (2006) FASEB J 20:500-502.
4 Marodon G, Mouly E, Blair E J, Frisen C, Lemoine F M, Klatzmann D (2003) Blood 101:3416-3423.
5 Zhang Y, Lu H, LiWang P, Sili U, Templeton N S (2003) Mol Ther 8:629-636.
6 Lai W, Chang C H, Farber D L (2003) J Immunol Methods 282:93-102.
7 Song E, Zhu P, Lee S K, Chowdhury D, Kussman S, Dykxhoorn D M, Feng Y, Palliser D, Weiner D B, Shankar P, et al. (2005) Nat Biotechnol 23:709-717.
8 Hutton J J, Jegga A G, Kong S, Gupta A, Ebert C, Williams S, Katz J D, Aronow B J (2004) BMC Genomics 5:82.
9 Staudt L M, Brown P O (2000) Annu Rev Immunol 18:829-859.
10 Shimaoka M, Xiao T, Liu J-H, Yang Y, Dong Y, Jun C-D, McCormack A, Zhang R, Joachimiak A, Takagi J, et al. (2003) Cell 112:99-111.
11 Kim M, Carman C V, Springer T A (2003) Science 301:1720-1725.
12 Carman C V, Springer T A (2003) Curr Opin Cell Biol 15:547-556.
13 Huang L, Shimaoka M, Rondon I J, Roy I, Chang Q, Po M, Dransfield D T, Ladner R C, Edge A S, Salas A, et al. (2006) J Leukocyte Biol 80:905-914.
14 Shimaoka M, Kim M, Cohen E, Yang W, Astrof N, Peer D, Salas A, Ferrand A, Springer T (2006) Proc Natl Acad Sci USA 103:13991-13996.
15 Wolfraim L A (2006) Arch Immunol Ther Exp (Warsz) 54:1-13.
16 Haskard D, Cavender D, Beatty P, Springer T A, Ziff M (1986) J Immunol 137:2901-2906.
17 Ma Q, Shimaoka M, Lu C, Jing H, Carman C V, Springer T A (2002) J Biol Chem 277:10638-10641.
18 Petruzzelli L, Maduzia L, Springer T A (1995) J Immunol 155:854-866.
19 Ma B, Liu W, Horner R J, Lee P J, Coyle A J, Lora J M, Lee C G, Elias J A (2006) J Immunol 176:49684978.
20 Berger E A, Murphy P M, Farber J M (1999) Annu Rev Immunol 17:657-700.
21 Ribeiro S, Horuk R (2005) Pharmacol Ther 107:4458.
22 Lu C, Springer T A (1997) J Immunol 159:268-278.
23 Arao T, Morimoto I, Kakinuma A, Ishida O, Zeki K, Tanaka Y, Ishikawa N, Ito K Eto S (2000) J Clin Endocrinol Metab 85: 382-389.
24 Tanaka Y, Mine S, Figdor C G, Wake A, Hirano H, Tsukada J, Aso M, Fujii K, Saito K, van Kooyk Y, Eto S (1998) Blood 91:3909-3919.
25 Goulvestre C, Chereau C, Nicco C, Mouthon L, Weill B, Batteux F (2005) J Immunol 175:69596967.
26 Shimaoka M, Lu C, Palframan R, von Andrian U H, McCormack A, Takagi J, Springer T A (2001) Proc Natl Acad Sci USA 98:6009-6014.
27 Dustin M L, Bivona T G, Philips M R (2004) Nat Immunol 5:363-372.
28 Hornung V, Guenthner-Biller M, Bourquin C, Ablasser A, Schlee M, Uematsu S, Noronha A, Manoharan M, Akira S, de Fougerolles A, et al. (2005) Nat Med 11:263-270.
29 Sioud M (2005) J Mol Biol 348:1079-1090.
30 Dustin M L, Springer T A (1989) Nature 341:619-624.
31 Constantin G, Majeed M, Giagulli C, Piccio L, Kim J Y, Butcher E C, Laudanna C (2000) Immunity 13:759-769.
32 Hargreaves R E, Monk N J, Jurcevic S (2004) Trends Mol Med 10: 130-135.
33 Sugamura K, Ishii N, Weinberg A D (2004) Nat Rev Immunol 4:420-431.
34 Soutschek J, Akinc A, Bramlage B, Charisse K, Constien R, Donoghue M, Elbashir S, Geick A, Hadwiger P, Harborth J, et al. (2004) Nature 432:173-178.
35 Morrissey D V, Lockridge J A, Shaw L, Blanchard K, Jensen K, Breen W, Hartsough K, Machemer L, Radka S, Jadhav V, et al. (2005) Nat Biotechnol 23:1002-1007.
36 Zimmermann T S, Lee A C, Akinc A, Bramlage B, Bumcrot D, Fedoruk M N, Harborth J, Heyes J A, Jeffs L B, John M, et al. (2006) Nature 441:111-114.
37 Kim M, Carman C V, Yang W, Salas A, Springer T A (2004) J Cell Biol 167:1241-1253.
38 Lu C, Shimaoka M, Salas A, Springer T A (2004) J Immunol 173:3972-3978.
39 Harlan J M, Winn R K (2002) Crit Care Med 30:S214-S219.
40 Lebwohl M, Tyring S K, Hamilton T K, Toth D, Glazer S, Tawfik N H, Walicke P, Dummer W, Wang X, Garovoy M R, et al. (2003) N Engl J Med 349:2004-2013.
41 de Fougerolles A R (2003) in I Domains in Integrins, ed Gullberg D (Plenum, Georgetown, Tex.), pp 165-177.

1. (canceled)

2. A leukocyte-selective delivery agent comprising,
(a) a targeting moiety that selectively binds LFA-I;
(b) a protein carrier moiety covalently linked to the targeting moiety; and
(c) a therapeutic agent associated with the carrier moiety.

3. The delivery agent of claim 2, which is further selective for activated leukocytes, wherein the targeting moiety selectively binds LFA-I in its activated conformation.

4. The delivery agent of claim 2, wherein the targeting moiety comprises an antibody or functional fragment thereof.

5. The delivery agent of claim 4, wherein the targeting moiety comprises an scFV.

6. The delivery agent of claim 4, wherein the antibody or functional fragment thereof binds to the locked open I domain of LFA-I, or binds to the leg domain of the β₂ subunit of LFA-I (α_{L}β₂).

7. The delivery agent of claim 5, wherein the antibody or functional fragment thereof binds non-selectively to both low affinity and high affinity LFA-I.

8. The delivery agent of claim 2, wherein the protein carrier moiety comprises a basic polypeptide.

9. The delivery agent of claim 8 wherein the basic polypeptide comprises protamine or a functional fragment thereof.

10. The delivery agent of claim 9, wherein the protein carrier moiety comprises the amino acid sequence RSQSRSRYYRQRQRSRRRRRRS (SEQ ID NO: 7).

11. The delivery agent of claim 2, wherein the therapeutic agent comprises one or more agents selected from the group consisting of a nucleic acid, a small molecule, a polypeptide, and an antibody or functional fragment thereof.

12. The delivery agent of claim 11 wherein the nucleic acid comprises an RNA interference molecule.

13. The delivery agent of claim 12 wherein the RNA interference molecule is selected from the group consisting of siRNA, dsRNA, stRNA, shRNA, miRNA, and combinations thereof.

14. The delivery agent of claim 13 wherein the therapeutic agent comprises CCR5-siRNA, ku70-siRNA, CD4-siRNA or cyclin-D1-siRNA.

15. The delivery agent of claim 12, wherein the nucleic acid comprises a small RNA, an antagomir, an LNA, or an antisense oligonucleotide.

16. A method for activated leukocyte-selective delivery comprising, administering to a subject an activated leukocyte-selective delivery agent comprising,
(a) a targeting moiety that selectively binds LFA-I in its activated conformation;
(b) a protein carrier moiety covalently linked to the targeting moiety; and
(c) a therapeutic agent associated with the carrier moiety;
to contact the delivery agent with activated leukocytes of the subject, to thereby selectively deliver the therapeutic agent to activated leukocytes of the subject.

17. The method of claim 16, wherein the subject has inappropriate leukocyte activation prior to administration of the delivery agent.

18. The method of claim 16 wherein the targeting moiety comprises an antibody or functional fragment thereof, which binds to the locked open I domain of LFA-I better than the locked closed I domain of LFA-i, or binds to the leg domain of the β₂ subunit of LFA-I (α_{L}β₂).

19. A method for in vivo leukocyte-selective delivery of a therapeutic agent, comprising, administering to a subject a leukocyte-selective delivery agent comprising,
(a) a targeting moiety that selectively binds LFA-I;
(b) a protein carrier moiety covalently linked to the targeting moiety; and
(c) a therapeutic agent associated with the carrier moiety; to contact the delivery agent with leukocytes of the subject, to thereby selectively deliver the therapeutic agent to leukocytes of the subject.

20. The method of claim 19 wherein the delivery agent is further selective for activated leukocytes, wherein the targeting moiety selectively binds LFA-I in its activated conformation.

21. The method of claim 19, wherein the targeting moiety comprises an antibody or functional fragment thereof.

22. The method of claim 21, wherein the antibody or functional fragment thereof comprises a scFV.

23. The method of claim 21, wherein the antibody or functional fragment thereof binds to the locked open I domain of LFA-I better than the locked closed I domain of LFA-I, or binds to the leg domain of the β₂ subunit of LFA-I (CILP₂).

24. The method of claim 21, wherein the antibody or functional fragment thereof binds non-selectively to both low affinity and high affinity LFA-I.

25. The method of claim 19, wherein the protein carrier moiety comprises a basic polypeptide.

26. The method of claim 25 wherein the basic polypeptide comprises protamine or a functional fragment thereof.

27. The method of claim 26, wherein the carrier moiety comprises the amino acid sequence RSQSRSRYYRQRQRSRRRRRRS (SEQ ID NO: 7).

28. The method of claim 19, wherein the therapeutic agent comprises one or more agents selected from the group consisting of a nucleic acid, a small molecule, a polypeptide, and an antibody or functional fragment thereof.

29. The delivery agent of claim 28 wherein the nucleic acid comprises an RNA interference molecule.

30. The method of claim 29, wherein the RNA interference molecule is selected from the group consisting of siRNA, dsRNA, stRNA, shRNA, miRNA, and combinations thereof.

31. The method of claim 30, wherein the siRNA comprises CCR5-siRNA, ku70-siRNA, CD4-siRNA or cyclin-D1-siRNA.

32. A method for leukocyte-selective delivery comprising:
a) providing a leukocyte-selective delivery agent comprising,
(1) a targeting moiety that selectively binds LFA-I;
(2) a protein carrier moiety covalently linked to the targeting moiety; and
(3) a therapeutic agent associated with the carrier moiety;
b) contacting the delivery agent to a population of cells comprising leukocytes, to thereby selectively deliver the therapeutic agent to leukocytes in the population of cells.

33. The method of claim 32 wherein the population of cells is obtained from a subject, and contacting step b) is performed in vitro.

34. The method of claim 33 further comprising administering the population of cells contacted with the delivery agent, to the subject.
